(19) United States
(12) Patent Application Publication
(10) Pub. No.: US 2025/0104806 A1
(43) Pub. Date: Mar. 27, 2025

(54) Detecting Cross-Contamination In Cell-Free RNA

(71) Applicant: GRAIL, LLC, Menlo Park, CA (US)

(72) Inventors: Ruth Mauntz, Redwood City, CA (US); Siddhartha Bagaria, San Francisco, CA (US); David Burkhardt, San Francisco, CA (US); Matthew H. Larson, San Francisco, CA (US); Monica Portela dos Santos Pimentel, San Jose, CA (US)

(21) Appl. No.: 18/832,502

(22) PCT Filed: Jan. 27, 2023
(86) PCT No.: PCT/US2023/061502

§ 371 Date: Jul. 23, 2024

Related U.S. Application Data
(60) Provisional application No. 63/304,503, filed on Jan. 28, 2022.

(51) Int. Cl. G16B 20/20; G16B 5/20; G16B 30/20

(52) U.S. Cl. CPC G16B 20/20; G16B 5/20; G16B 30/20

(57) ABSTRACT

The present disclosure relates to an improved method for analyzing sequencing data to detect cross-sample contamination in a test sample. Determining cross-contamination in a test sample can be informative for determining that the test sample will be less likely to correctly identify the presence of cancer in the subject. Pre-determined single nucleotide polymorphisms selected from: an allele present in a select database or a genotyping SNP associated with a sample type are used to identify. A sample is determined to be contaminated using the determined contamination probabilities of the one or more pre-determined SNPs.

### BACKGROUND

### 1. Field of Art

This application relates generally to detecting contamination in a sample, and more specifically to detecting contamination in a sample including targeted sequencing used for early detection of cancer.

### 2. Description of the Related Art

Next generation sequencing-based assays of circulating tumor DNA must achieve high sensitivity and specificity in order to detect cancer early. Early cancer detection and liquid biopsy both require highly sensitive methods to detect low tumor burden as well as specific methods to reduce false positive calls. Contaminating DNA from adjacent samples can compromise specificity which can result in false positive calls. In various instances, compromised specificity can be because rare SNPs from the contaminant may look like low level mutations. Methods currently exist for detecting and estimating contamination in whole genome sequencing data, typically from relatively low-depth sequencing studies. However, existing methods are not designed for detection of contamination in sequencing data from cancer detection samples, which typically require high-depth sequencing studies and include tumor-derived mutations (e.g., single base mutations and/or copy number variations (CNVs)) that may be present at varying frequencies (e.g., clonal and/or sub-clonal tumor-derived mutations). There is a need for new methods of detecting cross-sample contamination in sequencing data from a test sample used for cancer detection.

### SUMMMARY

Embodiments described herein relate to methods of analyzing sequencing data to detect cross-sample contamination in a test sample. Determining cross-contamination in a test sample can be informative for determining that the test sample will be less likely to correctly identify the presence of cancer in the subject. In one example, cross-contamination is determined in a nucleic acid sample obtained from a human subject and used for the early detection of cancer.

In various embodiments, samples (e.g., test samples) are obtained from subjects and prepared using genome sequencing techniques to generate sequencing reads representing a plurality of nucleic acid fragments from the sample, including cell-free RNA. The sequencing reads include a number of sequencing reads having one or more pre-determined SNPs that can be used to identify contamination in the sample. Identifying a sequencing read as having one or more pre-determined SNPs modifies the data set of the sequencing reads such that it can be more easily analyzed to determine contamination. In addition, pre-determining a SNP enables identification of types of contamination, while also increasing the confidence with which contamination can be identified and lowering the limit of detection. Sequencing reads having one or more of the pre-determined SNPs are identified and an observed allele frequency is determined. Contamination probabilities can be based on the observed allelic frequency for each of the one or more pre-determined SNPS within the sample. Determining whether the sample is contaminated relies, at least in part, on the contamination probabilities of the one or more pre-determined SNPs.

In some embodiments, to determine contamination, the system can apply a contamination model including at least one likelihood test to a sequencing read of the plurality of sequencing reads. Here, the likelihood test obtains a current contamination probability representing the likelihood that the sample (e.g., the plurality of sequencing reads) is contaminated.

In some embodiments, to determine contamination, the system can apply a contamination model including generating a noise model. Generally, SNPs of the sample (e.g., test sample) at a given site are expected to have a variant allele frequency that can be modeled as a function of the minor allele frequency for SNPs at that site in a population, a contamination level, and a noise level. In some cases, the model can include a probability function based on the minor allele frequencies. Therefore, when analyzing the test sample obtained from a subject, variations from the expected variant allele frequency can be determined utilizing regression modeling. Specifically, regression modeling can be used to determine a contamination level and its statistical significance based on the relationship between the variant allele frequency and the minor allele frequency for a given site. If the determined contamination level of the test sample is above a threshold contamination level and the determined contamination level is statistically significant, a contamination event can be called. Calling a contamination event can indicate that at least some sequences included in the test sample originate from a different subject.

In one aspect, this disclosure features a method for identifying contamination in a sample, comprising: obtaining a plurality of sequencing reads for a plurality of nucleic acid fragments isolated from a sample comprising cell-free RNA (cfRNA); identifying sequencing reads that comprise one or more pre-determined single nucleotide polymorphisms (SNPs), thereby determining an observed allele frequency for each pre-determined SNP in the plurality of sequencing reads, wherein each of the one or more pre-determined SNPs are selected from: an allele present in one or more selected databases; or a genotyping SNP associated with a sample type; and determining whether the sample is contaminated using a determined contamination probability of the one or more pre-determined SNPs.

In some embodiments, wherein the identified sequencing reads that comprise the one or more pre-determined SNPs comprise a sequencing depth of at least 10 reads per million mapped reads (RPM).

In some embodiments, the identified sequencing read comprising the one or more pre-determined SNPs each comprise an exonic sequence.

In some embodiments, the exonic sequence comprises an exon-exon junction.

In some embodiments, the allele present in one or more select databases comprises an allele present in a universal human reference database.

In some embodiments, the one or more pre-determined SNPs are selected from Table 1.

In some embodiments, the allele present in the one or more select databases comprises an allele present in a NCBI dbSNP database (Build 155) that has a reference allele frequency in a range between 0.2 and 0.7.

In some embodiments, the one or more pre-determined SNPs are selected from Table 2.

In some embodiments, the one or more pre-determined SNPs does not include a conversion type comprising: A>G; T>C; C>T; or G>A.

In some embodiments, the one or more pre-determined SNPs are selected from Table 3.

In some embodiments, the method further comprising determining a contamination probability for each pre-determined SNP using its observed allele frequency.

In some embodiments, the method further comprising identifying two or more pre-determined SNPs in the sequencing reads, thereby determining an observed allele frequency for each of the two or more pre-determined SNPs in the plurality of sequencing reads.

In some embodiments, the two or more pre-determined SNPs are selected from Table 1, Table 2, Table 3, or any combination thereof.

In some embodiments, the allele present in a Universal Human Reference (UHR) comprises an allele having a homozygous frequency of at least 75% in the UHR and a homozygous frequency of 5% or less in a human sample.

In some embodiments, the reference allele frequency is in a range between 0.3 and 0.7.

In some embodiments, the reference allele frequency comprises a MAF, a VAF, a sequencing depth, or any combination thereof.

In some embodiments, the reference allele frequency comprises a MAF, wherein the MAF is in a range between 0.3 and 0.7.

In some embodiments, the method further comprising filtering the sequences by removing sequencing reads comprising SNPs including no-calls prior to determining a contamination probability.

In some embodiments, filtering further comprises removing sequences having a SNP with a A>G; G>A; T>C; or C>T conversion.

In some embodiments, the observed allelic frequency comprises: a minor allele frequency (MAF), a variable allele frequency, a sequencing depth, a noise rate, or any combination thereof.

In some embodiments, the observed allelic frequency comprises a MAF indicating contamination.

In some embodiments, the MAF is 0.5 or greater.

In some embodiments, the method further comprising discarding the sample following a determination that the sample is contaminated.

In some embodiments, the method further comprising assessing a risk introduced by contamination and using the risk in determining whether the sample is discarded.

In some embodiments, the risk introduced by the contamination is determined in part by determining a likely source of contamination.

In some embodiments, determining the contamination source lowers the risk introduced by the contamination, and wherein not determining the contamination source increases the risk introduced by the contamination.

In some embodiments, the method further comprising applying a contamination model to the sequencing reads identified as having one or more pre-determined SNPs and an observed allele frequency in the plurality of sequencing reads.

In some embodiments, the contamination model comprises at least one likelihood test.

In some embodiments, one or more likelihood tests are applied to a sequencing read of the plurality of sequencing reads using the associated contamination probability, wherein each test to obtain a current contamination probability is indicative of whether the sequencing reads are contaminated.

In some embodiments, the method further comprising:

determining that the sequencing reads are contaminated based on the current contamination probability of the at least one test being above a threshold associated with the at least one test likelihood test.

In some embodiments, the method further comprising:

determining that the sequencing reads are contaminated based on the current contamination probability of at least two likelihood tests being above a threshold associated with the at least two likelihood tests.

In some embodiments, the at least one likelihood test maximizes a likelihood function, the likelihood function proportional to the probability of an event occurring in a data set given a variable.

In some embodiments, applying the at least one likelihood test of the contamination model comprises:

comparing a set of generated contaminated sequencing reads to a set of previously obtained non-contaminated sequencing reads to determine the contamination probability.

In some embodiments, applying at least one likelihood test of the contamination model comprises: generating a null hypothesis representing that the sequencing reads are not contaminated; generating a set of contamination hypotheses representing that the sequencing reads are contaminated, wherein each contamination hypothesis of the set of contamination hypotheses is contaminated at a different contamination level; and applying a likelihood ratio test between the set of contamination hypotheses and the null hypothesis, wherein the likelihood ratio test obtains the current contamination probability.

In some embodiments, applying the at least one likelihood test of the contamination model comprises: comparing a set of generated contaminated sequencing reads to an average of previously obtained sequencing reads to determine the contamination probability, wherein the contamination probability is associated with the likelihood that the sequencing reads are contaminated at a contamination level.

In some embodiments, applying at least one likelihood test of the contamination model comprises: generating a set of contamination hypotheses representing that the sequencing reads are contaminated, wherein each contamination hypothesis of the set of contamination hypotheses is contaminated at a different contamination level; generating a null hypothesis representing the mean minor allele frequency at a contamination level for a plurality of previously obtained sequencing reads, wherein the contamination level is associated with the contamination hypothesis most likely to be contaminated; and applying a likelihood ratio test between the set of contamination hypotheses and the null hypothesis, wherein the likelihood ratio test obtains the current contamination probability.

In some embodiments, the contamination model comprises generating a noise model.

In some embodiments, the noise model represents a measure of background noise in a subset of sequencing reads, and wherein the noise model is generated based on the subset of the sequencing reads.

In some embodiments, the method further comprising applying the contamination model to an identified sequencing read using the observed allele frequency of the one or more pre-determined SNPs in the identified sequencing reads and the generated noise model to obtain a confidence score representing a measure of the predicted contamination in the sequencing reads.

In some embodiments, the background noise is a population measure of allele frequency in the subset of sequencing reads.

In some embodiments, the background noise is representative of the static noise generated when sequencing a SNP.

In some embodiments, the subset of sequencing reads comprises SNPs from uncontaminated and healthy test samples.

In some embodiments, generating the noise model further comprises: determining a noise coefficient for each SNP of the subset of sequencing reads, wherein the noise coefficient predicts the expected noise level for each SNP.

In some embodiments, the noise model generated based on the subset of sequencing reads is additionally based on a sample type of the sequencing reads.

In some embodiments, when the confidence score is above a threshold the contamination model predicts that the sequencing reads are contaminated.

In some embodiments, the contamination model additionally includes a random error term.

In another aspect, this disclosure features a system for determining contamination in a sample, comprising: (a) a computer processor; and (b) a non-transitory computer-readable storage medium storing instructions that, when executed by the computer processor, cause the computer processor to perform steps of any of the methods described herein.

In another aspect, this disclosure features a method of predicting presence of a disease in a sample, comprising: obtaining a plurality of sequencing reads for a plurality of nucleic acid fragments isolated from a sample comprising cell-free RNA (cfRNA); identifying contamination in a sample using any of the methods of described herein; and identifying SNPs from the plurality of sequencing reads that are informative for the presence of a disease.

In some embodiments, the method further comprising assessing the risk introduced by contamination identified in step (b).

In some embodiments, the risk introduced by the contamination is determined in part by determining a likely source of contamination.

In some embodiments, determining the contamination source lowers the risk introduced by the contamination, and wherein not determining the contamination source increases the risk introduced by the contamination.

In some embodiments, a contaminated sample is discarded based in part on the presence of contamination, the risk introduced by the contamination, or both.

In some embodiments, the disease is cancer.

### BRIEF DESCRIPTION OF DRAWINGS

FIG. 1 is a flowchart of a method for preparing a nucleic acid sample for sequencing, according to one example embodiment.

FIG. 2 is a block diagram of a processing system for processing sequence reads, according to one example embodiment.

FIG. 3 is a flowchart of a method for determining variants of sequence reads, according to one example embodiment.

FIG. 4 shows an error plot with mean error rate (y-axis) plotted against mean sequencing depth (x-axis), according to one example embodiment.

FIGS. 5A-5B show histograms for error rate (y-axis) for each of the different conversion types (x-axis), according to one example embodiment. FIG. 5A shows error rate (y-axis) for each of the different conversion types (x-axis) when analyzing SNPs from whole transcriptome data. FIG. 5B shows error rate (y-axis) for each of the different conversion types (x-axis) when analyzing SNPs from targeted panels. Error rate=alt counts/depth for each error mode in a sample.

FIG. 6 illustrates a flow diagram of a workflow for detecting contamination in a plurality of sequencing reads using contamination probabilities for one or more pre-determined SNPs, according to one example embodiment.

FIG. 7. illustrates a flow diagram of a workflow for detecting contamination in a plurality of sequencing reads using likelihood tests based on prior probabilities of contamination for one or more pre-determined SNPs, according to one example embodiment.

FIG. 8A illustrates a limit of detection workflow, according to one example embodiment.

FIG. 8B shows the limit of detection for the workflow of FIG. 8A.

FIG. 9A is a plot showing the analytical validation for limit of detection for cfRNA contamination, according to one example embodiment.

FIG. 9B shows the limit of detection for the workflow FIG. 8A.

FIG. 10A is a plot showing the analytical validation for limit of detection of UHR contamination, according to one example embodiment.

FIG. 10B shows the limit of detection for workflow FIG. 8A.

FIG. 11 illustrates a workflow of a method of validating the contamination detection application, according to one embodiment, according to one example embodiment.

FIG. 12A illustrates a workflow for in silico validation, according to one example embodiment.

FIG. 12B is a contamination estimation plot showing in silico validation, according to one example embodiment.

FIG. 12C shows contamination fraction (y-axis) plotted against average likelihood (Log) showing in silico validation when analyzing SNPs from targeted panels.

FIG. 12D shows contamination fraction (y-axis) plotted against average likelihood (Log) showing in silico validation when analyzing SNPs from whole transcriptome data.

FIG. 13 illustrates a block diagram of a contamination detection application for detecting and calling contamination in a plurality of sequence reads, according to one example embodiment. Dashed lines indicate optional workflow.

FIG. 14 illustrates a block diagram of a contamination detection application for detecting and calling contamination in a plurality of sequence reads, according to one example embodiment. Dashed lines indicate optional workflow.

The figures depict embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### I. Definitions

The term “individual” refers to a human individual. The term “healthy individual” refers to an individual presumed to not have cancer or disease. The term “subject” refers to an individual who is known to have, or potentially has, cancer or disease.

The term “sample” refers to a biological specimen taken from an individual or subject. Sample can refer to one or more samples taken from an individual or subject and combined prior to performing the detection methods described herein. For example, genome sequencing techniques commonly combine samples prior to performing a sequencing reaction. In such cases, the samples are labeled prior to combining. Sample can refer to nucleic acid fragments taken from targeted panels. Sample can refer to nucleic acid fragments taken from whole transcriptome and/or whole genome data.

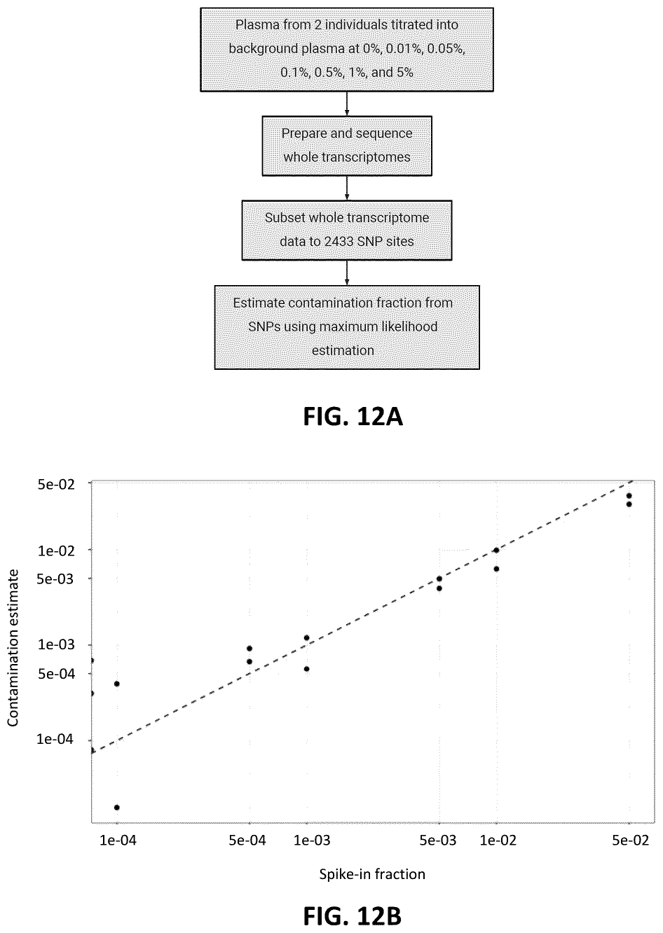

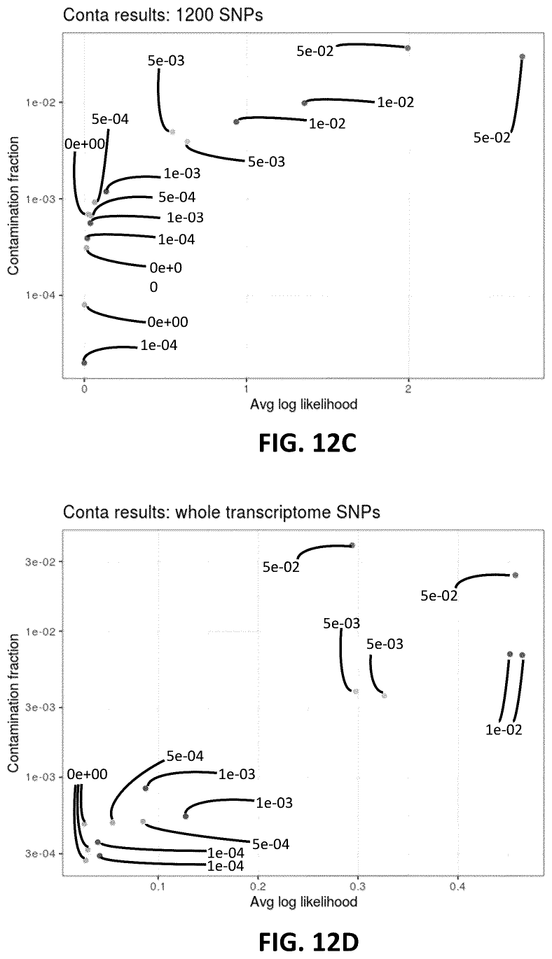

FIG. 12D shows contamination fraction (y-axis) plotted against average likelihood (Log) showing in silico validation when analyzing SNPs from whole transcriptome data

The term “sequence reads” or “sequencing reads” refers to nucleotide sequences read obtained from a sample. Sequence reads can be obtained through various methods known in the art.

The term “a plurality of sequencing reads” refers to all or a portion of a plurality of nucleic acid sequences or fragments from a sample.

The term “read segment” or “read” refers to any nucleotide sequences including sequence reads obtained from an individual and/or nucleotide sequences derived from the initial sequence read from a sample obtained from an individual. For example, a read segment can refer to an aligned sequence read, a collapsed sequence read, or a stitched read. Furthermore, a read segment can refer to an individual nucleotide base, such as a single nucleotide variant.

The term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

The term “single nucleotide polymorphism” or “SNP” refers to a substitution of one nucleotide to a different nucleotide at a position (e.g., site) of a nucleotide sequence, e.g., a sequence read from an individual. For example, at a specific base site, the nucleobase C may appear in most individuals, but in a minority of individuals, the position is occupied by base A. There is a SNP at this specific site.

The term “pre-determined single nucleotide polymorphism” or “pre-determined SNP” refers to a SNP identified prior to performing any of the methods described herein (e.g., prior identifying sequencing reads). For example, a pre-determined SNP is identified prior to identifying sequence reads that comprises one or more pre-determined single nucleotide polymorphisms. A pre-determined SNP, alone or in combination with one or more additional pre-determined SNPs, enables identification of contamination in a sample.

The term “indel” refers to any insertion or deletion of one or more base pairs having a length and a position (which may also be referred to as an anchor position) in a sequence read. An insertion corresponds to a positive length, while a deletion corresponds to a negative length.

The term “mutation” refers to one or more SNVs or indels.

The term “true positive” refers to a mutation that indicates real biology, for example, the presence of potential cancer, disease, or germline mutation in an individual. True positives are not caused by mutations naturally occurring in healthy individuals (e.g., recurrent mutations) or other sources of artifacts such as process errors during assay preparation of nucleic acid samples.

The term “false positive” refers to a mutation incorrectly determined to be a true positive. Generally, false positives may be more likely to occur when processing sequence reads associated with greater mean noise rates or greater uncertainty in noise rates.

The term “cell-free nucleic acid,” “cell-free DNA,” “cfDNA,” “cell-free RNA,” or “cfRNA” refers to nucleic acid fragments that circulate in an individual's body (e.g., bloodstream) and originate from one or more healthy cells and/or from one or more cancer cells. A sample, as described herein, can include cell-free nucleic acids (e.g., cfRNA).

The term “circulating tumor DNA” or “ctDNA” refers to nucleic acid fragments that originate from tumor cells or other types of cancer cells, which may be released into an individual's bloodstream as result of biological processes such as apoptosis or necrosis of dying cells or actively released by viable tumor cells. Nucleic acid fragments that originate from tumor cells or other types of cancer cells can be informative of the presence or absence of cancer (or disease), cancer status, or a cancer classification (e.g., cancer type or tissue of origin).

The term “genomic nucleic acid,” “genomic DNA,” or “gDNA” refers to nucleic acid including chromosomal DNA that originates from one or more healthy cells.

The term “alternative allele” or “ALT” refers to an allele having one or more mutations relative to a reference allele, e.g., corresponding to a known gene.

The term “minor allele” or “MIN” refers to the second most common allele in a given population.

The term “sequencing depth” or “depth” refers to a total number of read segments from a sample obtained from an individual that have a particular location in the genome. A non-limiting example of sequencing depth described herein includes “reads per million” (RPM) mapped reads.

The term “allele depth” or “AD” refers to a number of read segments in a sample that supports an allele in a population. The terms “AAD”, “MAD” refer to the “alternate allele depth” (i.e., the number of read segments that support an ALT) and “minor allele depth” (i.e., the number of read segments that support a MIN), respectively.

The term “contaminated” refers to a test sample that is contaminated with at least some portion of a second test sample. That is, a contaminated test sample unintentionally includes DNA sequences from an individual that did not generate the test sample. Similarly, the term “uncontaminated” refers to a test sample that does not include at least some portion of a second test sample.

The term “contamination level” refers to the degree of contamination in a test sample. That is, the contamination level the number of reads in a first test sample from a second test sample. For example, if a first test sample of 1000 reads includes 30 reads from a second test sample, the contamination level is 3.0%.

The term “contamination event” refers to a test sample being called contaminated. Generally, a test sample is called contaminated if the determined contamination level is above a threshold contamination level and the determined contamination level is statistically significant.

The term “allele frequency” or “AF” refers to the frequency of a given allele in a population. The terms “AAF”, “MAF” refer to the “alternate allele frequency” and “minor allele frequency”, respectively. Herein, the term “variant allele frequency” refers to the minor allele frequency for an allele of the test sample. In this case, the VAF may be determined by dividing the corresponding variant allele depth of a test sample by the total depth of the sample for the given allele.

The term “reference allele frequency” refers to the frequency of a given allele in a previously sequenced sample. For example, a reference allele frequency refers to allele frequency for an allele in a previously sequenced sample that included cfRNA where allele frequency was determined. In another example, the reference allele frequency refers to allele frequency for an allele in a NCBI dbSNP database (Build 155).

The term “observed allele frequency” refers to frequency of a given allele in a sample where the detection methods described herein were used, at least in part, to determine the allele frequency. An observed allele frequency can be then used to determine where the sample is contaminated.

### II. Detecting Contamination Based on Pre-Determined Snps

In various embodiments, samples (e.g., test samples) are obtained from subjects and prepared using genome sequencing techniques to generate sequencing reads representing a plurality of nucleic acid fragments from the sample, including cell-free RNA. The sequencing reads include a number of sequencing reads having one or more pre-determined SNPs that can be used to identify contamination in the sample. Identifying a sequencing read as having one or more pre-determined SNPs modifies the data set of the sequencing reads such that it can be more easily analyzed to determine contamination. In addition, pre-determining a SNP enables identification of types of contamination, while also increasing the confidence with which contamination can be identified and lowering the limit of detection. Sequencing reads having one or more of the pre-determined SNPs are identified and an observed allele frequency is determined. Contamination probabilities can be based on the observed allelic frequency for each of the one or more pre-determined SNPS within the sample. Determining whether the sample is contaminated relies, at least in part, on the contamination probabilities of the one or more pre-determined SNPs. In some embodiments, to determine contamination, the system can apply a contamination model including at least one likelihood test to a sequencing read of the plurality of sequencing reads. Here, the likelihood test obtains a current contamination probability representing the likelihood that the sample (e.g., the plurality of sequencing reads) is contaminated.

### II.A. Example Assay Protocol

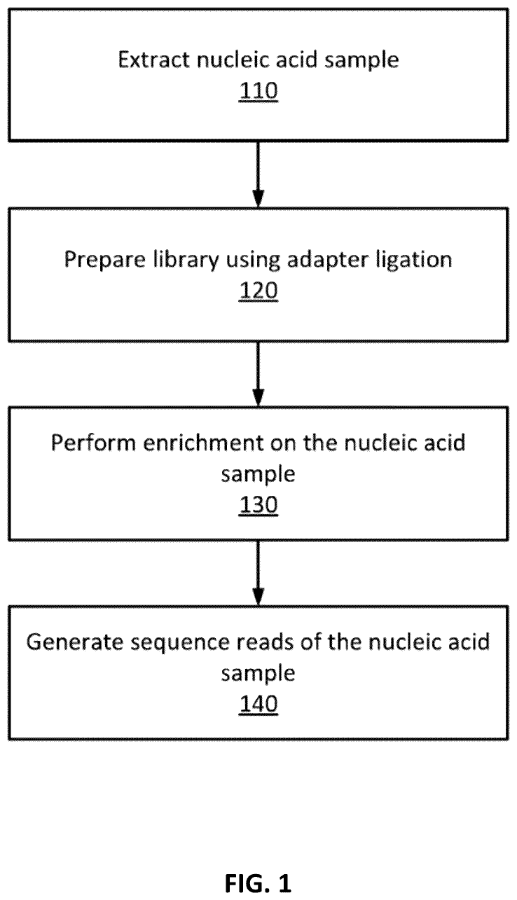

FIG. 1 is a flowchart of a method 100 for preparing a nucleic acid sample for sequencing according to one embodiment. The method 100 includes, but is not limited to, the following steps. For example, any step of the method 100 may comprise a quantitation sub-step for quality control or other laboratory assay procedures known to one skilled in the art.

In step 110, a nucleic acid sample (DNA or RNA) is extracted from a subject. In the present disclosure, DNA and RNA may be used interchangeably unless otherwise indicated. That is, the following embodiments for using error source information in variant calling and quality control may be applicable to both DNA and RNA types of nucleic acid sequences. However, the examples described herein may focus on DNA for purposes of clarity and explanation. The sample may be any subset of the human genome, including the whole genome. The sample may be extracted from a subject known to have or suspected of having cancer. The sample may include blood, plasma, serum, urine, fecal, saliva, other types of bodily fluids, or any combination thereof. In some embodiments, methods for drawing a blood sample (e.g., syringe or finger prick) may be less invasive than procedures for obtaining a tissue biopsy, which may require surgery. The extracted sample may comprise cfDNA and/or ctDNA. For healthy individuals, the human body may naturally clear out cfDNA and other cellular debris. If a subject has cancer or disease, ctDNA in an extracted sample may be present at a detectable level for diagnosis.

In step 120, a sequencing library is prepared. During library preparation, unique molecular identifiers (UMI) are added to the nucleic acid molecules (e.g., DNA molecules) through adapter ligation. The UMIs are short nucleic acid sequences (e.g., 4-10 base pairs) that are added to ends of DNA fragments during adapter ligation. In some embodiments, UMIs are degenerate base pairs that serve as a unique tag that can be used to identify sequence reads originating from a specific DNA fragment. During PCR amplification following adapter ligation, the UMIs are replicated along with the attached DNA fragment, which provides a way to identify sequence reads that came from the same original fragment in downstream analysis.

In step 130, targeted DNA sequences are enriched from the library. During enrichment, hybridization probes (also referred to herein as “probes”) are used to target, and pull down, nucleic acid fragments informative for the presence or absence of cancer (or disease), cancer status, or a cancer classification (e.g., cancer type or tissue of origin). For a given workflow, the probes may be designed to anneal (or hybridize) to a target (complementary) strand of DNA or RNA. The target strand may be the “positive” strand (e.g., the strand transcribed into mRNA, and subsequently translated into a protein) or the complementary “negative” strand. The probes may range in length from 10s, 100s, or 1000s of base pairs. In one embodiment, the probes are designed based on a gene panel to analyze particular mutations or target regions of the genome (e.g., of the human or another organism) that are suspected to correspond to certain cancers or other types of diseases. Moreover, the probes may cover overlapping portions of a target region. By using a targeted gene panel rather than sequencing all expressed genes of a genome, also known as “whole exome sequencing,” the method 100 may be used to increase sequencing depth of the target regions, where depth refers to the count of the number of times a given target sequence within the sample has been sequenced. Increasing sequencing depth reduces required input amounts of the nucleic acid sample. After a hybridization step, the hybridized nucleic acid fragments are captured and may also be amplified using PCR.

In step 140, sequence reads are generated from the enriched DNA sequences. Sequencing data may be acquired from the enriched DNA sequences by known means in the art. For example, the method 100 may include next-generation sequencing (NGS) techniques including synthesis technology (Illumina), pyrosequencing (454 Life Sciences), ion semiconductor technology (Ion Torrent sequencing), single-molecule real-time sequencing (Pacific Biosciences), sequencing by ligation (SOLID sequencing), nanopore sequencing (Oxford Nanopore Technologies), or paired-end sequencing. In some embodiments, massively parallel sequencing is performed using sequencing-by-synthesis with reversible dye terminators.

In some embodiments, the sequence reads may be aligned to a reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene.

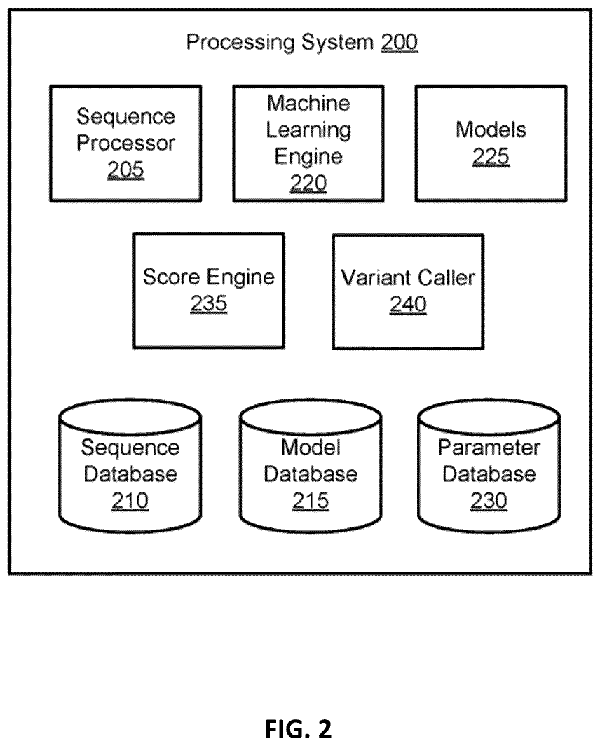

In various embodiments, a sequence read is comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome represent the likely location within the reference genome to which the nucleic acid fragment corresponds. An output file having SAM (sequence alignment map) format or BAM (binary) format may be generated and output for further analysis such as variant calling, as described below with respect to FIG. 2.

### II.B. Example Processing System

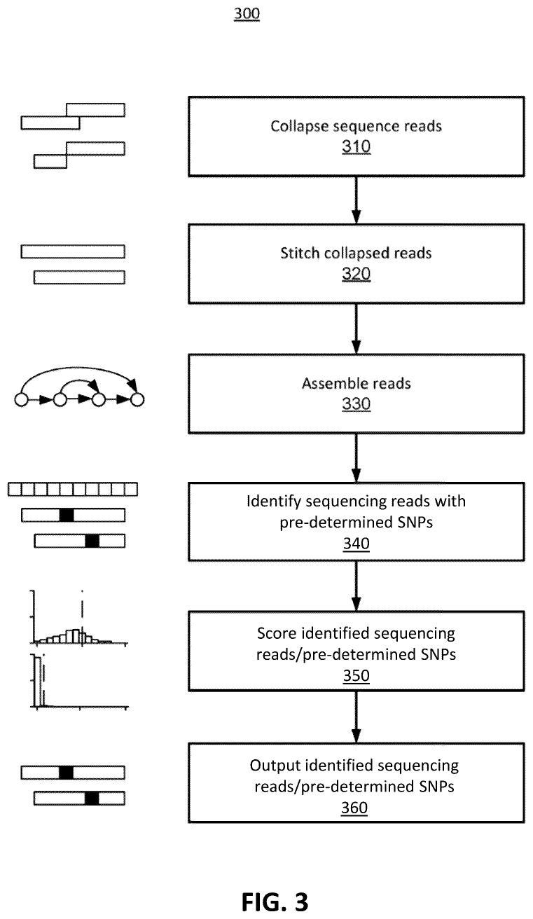

FIG. 2 is a block diagram of a processing system 200 for processing sequence reads, according to one example embodiment. The processing system 200 includes a sequence processor 205, sequence database 210, model database 215, machine learning engine 220, models 225, parameter database 230, score engine 235, variant caller 240 and copy number variation (CNV) caller (not pictured). FIG. 3 is a flowchart of a method 300 for determining variants (e.g., a SNP and/or a pre-determine SNP) in a sequencing read from a plurality of sequencing reads, according to one example embodiment. In some embodiments, the processing system 200 performs the method 300 to perform variant calling (e.g., for SNPs) based on input sequencing data. Further, the processing system 200 may obtain the input sequencing data from an output file associated with a nucleic acid sample (e.g., a plurality of nucleic acid fragments isolated from a sample comprising cell-free RNA (cfRNA)) prepared using the method 100 described above. The method 300 includes, but is not limited to, the following steps, which are described with respect to the components of the processing system 200. In other embodiments, one or more steps of the method 300 may be replaced by a step of a different process for generating variant calls, e.g., using Variant Call Format (VCF), such as HaplotypeCaller, VarScan, Strelka, or SomaticSniper.

The processing system 200 can be any type of computing device that is capable of running program instructions. Examples of processing system 200 may include, but are not limited to, a desktop computer, a laptop computer, a tablet device, a personal digital assistant (PDA), a mobile phone or smartphone, and the like. In one example, when processing system is a desktop or laptop computer, models 225 may be executed by a desktop application. Applications can, in other examples, be a mobile application or web-based application configured to execute the models 225.

At step 310, the sequence processor 205 collapses aligned sequence reads of the input sequencing data. In one embodiment, collapsing sequence reads includes using UMIs, and optionally alignment position information from sequencing data of an output file (e.g., from the method 100 shown in FIG. 1) to collapse multiple sequence reads into a consensus sequence for determining the most likely sequence of a nucleic acid fragment or a portion thereof. Since the UMIs are replicated with the ligated nucleic acid fragments through enrichment and PCR, the sequence processor 205 may determine that certain sequence reads originated from the same molecule in a nucleic acid sample. In some embodiments, sequence reads that have the same or similar alignment position information (e.g., beginning and end positions within a threshold offset) and include a common UMI are collapsed, and the sequence processor 205 generates a collapsed read (also referred to herein as a consensus read) to represent the nucleic acid fragment. The sequence processor 205 designates a consensus read as “duplex” if the corresponding pair of collapsed reads have a common UMI, which indicates that both positive and negative strands of the originating nucleic acid molecule are captured; otherwise, the collapsed read is designated “non-duplex.” In some embodiments, the sequence processor 205 may perform other types of error correction on sequence reads as an alternative to, or in addition to, collapsing sequence reads.

At step 320, the sequence processor 205 stitches the collapsed reads based on the corresponding alignment position information. In some embodiments, the sequence processor 205 compares alignment position information between a first read and a second read to determine whether nucleotide base pairs of the first and second reads overlap in the reference genome. In one use case, responsive to determining that an overlap (e.g., of a given number of nucleotide bases) between the first and second reads is greater than a threshold length (e.g., threshold number of nucleotide bases), the sequence processor 205 designates the first and second reads as “stitched”; otherwise, the collapsed reads are designated “unstitched.” In some embodiments, a first and second read are stitched if the overlap is greater than the threshold length and if the overlap is not a sliding overlap. For example, a sliding overlap may include a homopolymer run (e.g., a single repeating nucleotide base), a dinucleotide run (e.g., two-nucleotide base sequence), or a trinucleotide run (e.g., three-nucleotide base sequence), where the homopolymer run, dinucleotide run, or trinucleotide run has at least a threshold length of base pairs.

At step 330, the sequence processor 205 assembles reads into paths. In some embodiments, the sequence processor 205 assembles reads to generate a directed graph, for example, a de Bruijn graph, for a target region (e.g., a gene). Unidirectional edges of the directed graph represent sequences of k nucleotide bases (also referred to herein as “k-mers”) in the target region, and the edges are connected by vertices (or nodes). The sequence processor 205 aligns collapsed reads to a directed graph such that any of the collapsed reads may be represented in order by a subset of the edges and corresponding vertices.

At step 340, the variant caller 240 identifies sequencing reads that include one or more pre-determined SNPs from the paths assembled by the sequence processor 205. In one embodiment, the variant caller 240 identifies sequencing reads that include one or more pre-determined SNPs by comparing a directed graph (which may have been compressed by pruning edges or nodes in step 310) to a reference sequence of a target region of a genome or a reference sequence that includes one or more of the pre-determined SNPs (e.g., obtained sequencing reads from a sequence UHR or sample that includes cfRNA). The variant caller 240 may align edges of the directed graph to the reference sequence and record the genomic positions of mismatched edges and mismatched nucleotide bases adjacent to the edges as the locations of candidate variants. Additionally, the variant caller 240 may identify sequencing reads that including one or more pre-determined SNPs based on the sequencing depth of a target region. In particular, the variant caller 240 may be more confident in identifying sequencing reads that include one or more pre-determined SNPs in target regions that have greater sequencing depth, for example, because a greater number of sequence reads help to resolve (e.g., using redundancies) mismatches or other base pair variations between sequences.

Further, multiple different models may be stored in the model database 215 or retrieved for application post-training. For example, models may be trained to determine the presence of a contamination event (e.g., contamination of a test sample during process 100 or process 300) and/or verify contamination detection. Further, the score engine 235 may use parameters of the model 225 to determine a likelihood of one or more true positives or contamination in a sequence read. The score engine 235 may determine a quality score (e.g., on a logarithmic scale) based on the likelihood. For example, the quality score is a Phred quality score Q=−10·log₁₀ P, where P is the likelihood of an incorrect candidate variant call (e.g., a false positive). In some embodiments, CNV caller 240 can call copy number variations using a model stored in the model database 215. In one example, CNVs associated with one or more pre-determined SNPs are identified using a model that analyzes the presence or absence of one or more of the pre-determined SNPs. In one example, CNVs associated with cancer are identified using a model that analyzes random sequencing data. In another example, CNVs associated with cancer are identified using a model that analyzes allele ratios at a plurality of heterozygous loci within a region of the genome.

At step 350, the score engine 235 scores the identified sequencing reads and/or the pre-determined SNPs based on the model 225 (e.g., the presence or absence of the one or more pre-determined SNPs) or corresponding likelihoods of true positives, contamination, quality scores, etc. Training and application of the model 225 are described in more detail below.

At step 360, the processing system 200 outputs the identified sequencing reads and/or the pre-determined SNPs. In some embodiments, the processing system 200 outputs some or all of the identified sequencing reads and/or pre-determined SNP along with the corresponding scores. Downstream systems, e.g., external to the processing system 200 or other components of the processing system 200, may use the pre-determined SNPs and scores for various applications including, but not limited to, predicting the presence of cancer, predicting contamination in test sequences, or predicting noise levels.

### II.C. Using Pre-Determined SNPs

In one aspect this disclosure features methods for identifying contamination in a sample where the method includes: (a) obtaining a plurality of sequencing reads for a plurality of nucleic acid fragments isolated from a sample comprising cell-free RNA (cfRNA); (b) identifying sequencing reads that comprise one or more pre-determined single nucleotide polymorphisms (SNPs) thereby determining an observed allele frequency for each pre-determined SNP in the plurality of sequencing reads, and wherein each of the one or more pre-determined SNPs are selected from: (i) an allele present in a Universal Human Reference (UHR) database; (ii) an allele present in a NCBI dbSNP database (Build 155) that has a reference allele frequency in a range between 0.3 and 0.7; and (iii) a genotyping SNP associated with a sample type; and (c) determining whether the sample is contaminated using the determined contamination probabilities of the one or more pre-determined SNPs. In some embodiments, the methods provided herein further comprise determining a contamination probability for each pre-determined SNP using its observed allele frequency and determining whether the sample is contaminated using the determined contamination probabilities of the one or more pre-determined SNPs.

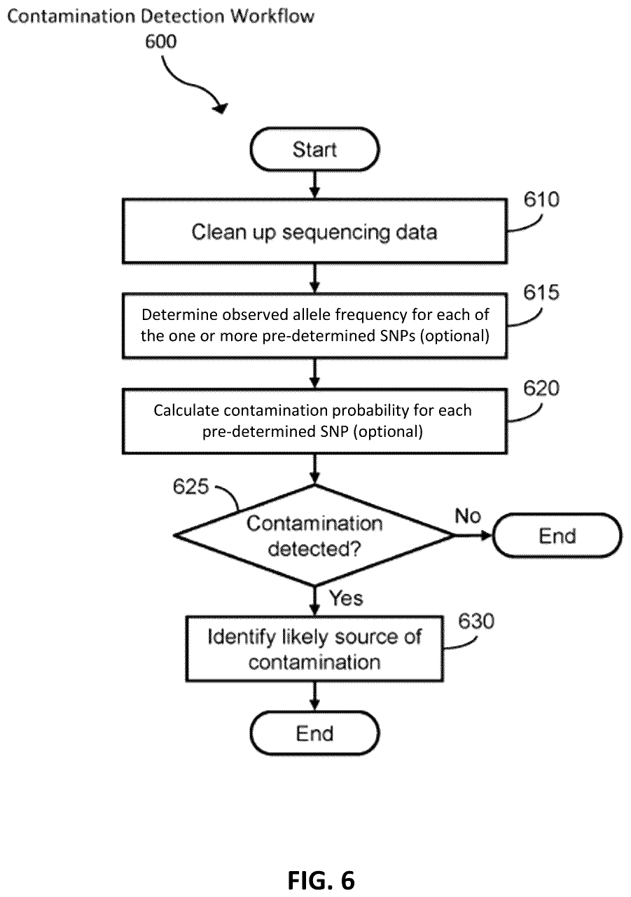

In a non-limiting example, FIG. 6 provides a flow diagram illustrating a contamination detection workflow 600. In some embodiments, the workflow of 600 is executed on the processing system 200. The detection workflow 600 of this embodiment includes, but is not limited to, the following steps.

At step 610, sequencing data obtained from a sample (e.g., using the process 300) is cleaned up. For example, data cleaning may include removing a pre-determined SNP with: no coverage, a sequencing depth less than a threshold (e.g., any of the sequence depth thresholds described herein), a high error frequency (e.g., >0.1%), high variance, and/or a particular genomic location (e.g., when the SNP is present within an intron or other non-coding region).

At step 615, optionally, observed allele frequencies for each of the one or more pre-determined SNPs are determined.

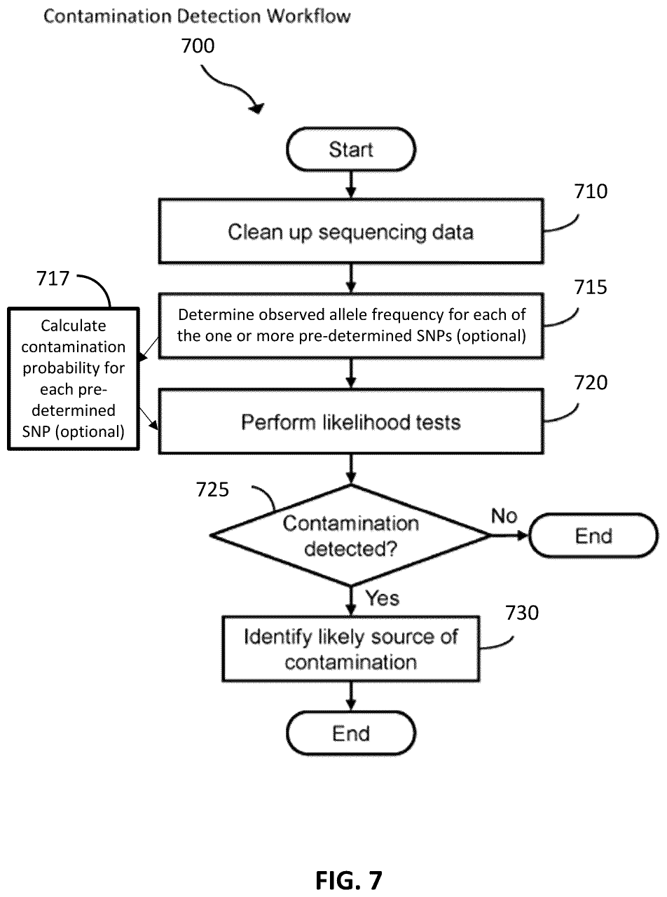

At step 620, optionally, a contamination probability for each of the one or more pre-determined SNPs using its observed allele frequency is calculated. In some cases, step 620 includes applying a contamination model to the sequencing reads identified as having one or more pre-determined SNPs and an observed allele frequency in the plurality of sequencing reads. In one embodiment, method 600 also includes applying a contamination model that includes performing likelihood tests based, at least in part, on the observed allele frequencies for each of the one or more pre-determined SNPs identified in the sample (see, e.g., FIG. 7). In another embodiment, method 600 also includes applying a contamination model that includes generating a noise model analysis as described herein.

At step 625, a determination is made whether or not the sample is contaminated using the determined contamination probabilities of the one or more pre-determined SNPs. In one embodiment, at decision step 625, it is determined whether the plurality of sequencing reads are contaminated. If the plurality of sequencing reads have an observed allele frequencies at one or more of the pre-determined SNPs that identify contamination is present, then the sample is contaminated and workflow 600 proceeds to a step 630. If a plurality of sequencing reads does not have an observed allele frequency at the one or more pre-determined SNPs that identify contamination is present, then the sample is not contaminated and workflow 600 ends.

At step 630, a likely source of contamination is identified. In one embodiment, a genotyping SNP (e.g., a genotyping SNP as described herein, e.g., in Table 1) is used to identify the source of contamination. In another embodiment, contamination is identified based on the prior probabilities of SNPs from known genotypes of other samples that were processed in the same batch as the test sample (or a set of related batches).

### III. Selecting Pre-Determined Single Nucleotide Polymorphisms

In one aspect, this disclosure features methods for identifying contamination in a sample where the method includes identifying one or more pre-determined single nucleotide polymorphisms (SNPs) prior to determining contamination. A SNP can be considered a “pre-determined SNP” based, at least in part, on its ability to aid in the determination of whether a sample is contaminated. In some embodiments, a pre-determined SNP is selected based on one or more of the following: an allele present in one or more selected databases; or a genotyping SNP associated with a sample type. In some embodiments, a pre-determined SNP is selected based on one or more of the following: (i) an allele present in a universal human reference database; (ii) an allele present in a NCBI dbSNP database (Build 155) that has a reference allele frequency in a range between 0.2 and 0.8 (or any of the subranges therein); and/or (iii) a genotyping SNP associated with a sample type.

In some embodiments, the steps of selecting a pre-determined SNP to be included in the contamination detection method occurs prior to obtaining a plurality of sequencing reads for a plurality of nucleic acid fragments isolated from a sample comprising cell-free RNA (cfRNA) or after obtaining the plurality of sequencing reads. In some embodiments, one or more pre-determined SNPs are selected based on the outputs of one or more of the steps related to method 300. For example, a SNP is selected as a pre-determined SNP, based, at least in part, on the sequencing depth determined after step 320. In another example, a SNP is selected, based, at least in part, on the statistical significance associated with the paths assembled in step 330.

In some embodiments, one or more pre-determined SNPs can be removed/filtered out based, at least in part, on the outputs of one or more of the steps related to the method 300. For example, a SNP is not selected (e.g., removed or filtered out) as a pre-determined SNP based, at least in part, on the sequencing depth determined after step 320. In another example, a SNP is not selected (e.g., removed or filtered out) as a pre-determined SNP based, at least in part, on the statistical significance associated with the paths assembled in step 330.

Additional criteria can be used to select a SNP as a pre-determined SNP. Non-limiting examples of additional criteria include: observed sequencing depth in previously sequenced samples, low error rates in previously sequence samples, and genomic location (e.g., a sequencing read including all or a portion of an exonic sequence).

In some embodiments, the method is premised in part on obtaining sequencing reads (e.g., a sequencing read identified as having one or more pre-determined SNPs) sequenced at sufficient sequencing depth to enable contamination detection. For example, a pre-determined SNP has sufficient sequencing depth when at least 25 sequencing reads (e.g., at least 50 sequencing reads, at least 75 sequencing reads, at least 100 sequencing reads, at least 125 sequencing reads, at least 150 sequencing reads, at least 175 sequencing reads, or at least 200 sequencing reads) map to the genomic location of the pre-determined SNP. In some embodiments, a pre-determined SNP has sufficient sequencing depth when the samples has a sequencing depth of at least 10 reads per million mapped reads (RPM), at least 25 RPM, at least 50 RPM, at least 100 RPM, at least 500 RPM, or at least 1000 RPM in the plurality of sequencing reads (or sample).

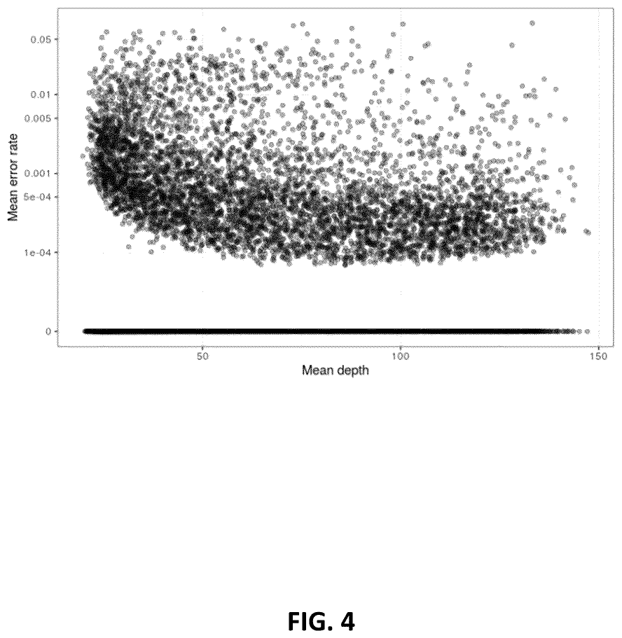

As shown in FIG. 4, high error rates correlate with low sequencing depth. FIG. 4 shows 50,000 candidate dbSNPs having wild-type (WT) noncancer expression, sequencing depth between 15 sequencing reads and 150 sequence reads, and a minor allele frequency (MAF) of 0.3<MAF<0.7. Reads with low sequencing depth had higher error rates, including error rates above the assay error rate between about 10-4 to about 10-3 described herein. As such, pre-determined SNPs present at a genomic locus that have a sequencing depth below a threshold (e.g., any of the sequencing depth criteria described herein) are excluded due to high error rates.

In some embodiments, a pre-determined SNP comprises a low error rate when detected in the plasma cfRNA. Low error rates enable a pre-determined SNP to be distinguished from technical errors from trace contamination events arising from or during performance of the assay.

In some embodiments, a pre-determined SNP is present in an exon. In some embodiments, a sequencing read identified as having one or more pre-determined SNPs is excluded if the sequencing read does not include all or a portion of an exonic sequence. In some embodiments, a sequencing read identified as having one or more pre-determined SNPs and including all or a portion of an exonic sequence results in greater statistical significance being assigned to paths assembled in step 330. In some embodiments, a sequencing read identified as having one or more pre-determined SNPs is given greater weight (e.g., a contamination model is adjusted to weight the presence of the pre-determined SNP more heavily) if the sequencing read includes all or a portion of an exonic sequence (e.g., an exon-exon junction).

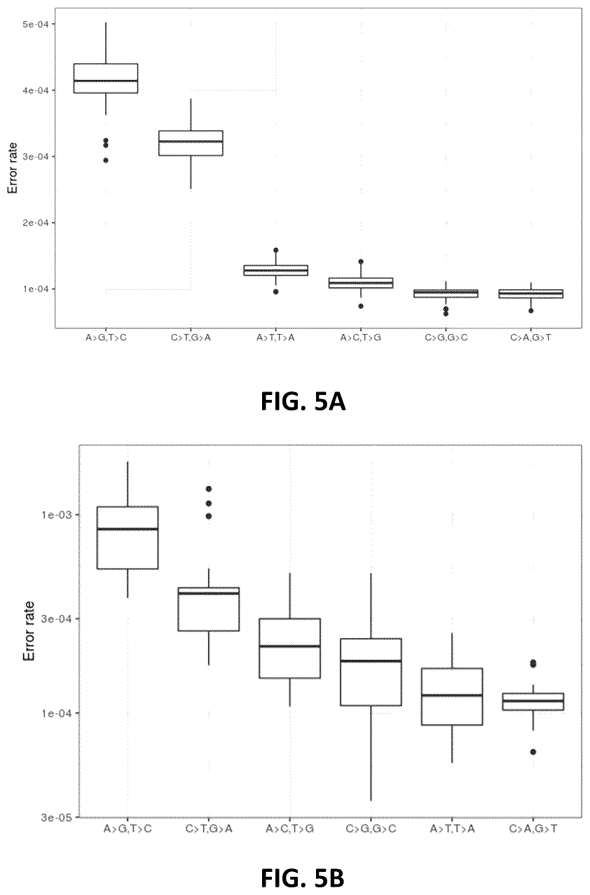

In some embodiments, one or more of the predetermined SNPs do not include SNPs having a conversion type comprising: A>G; T>C; C>T; or G>A. Conversion types including A>G; T>C; C>T; or G>A can be difficult to differentiate from low-level contamination events (See, e.g., FIGS. 5A-5B). In some embodiments, a pre-determined SNP having a conversion type comprising A>G; T>C; C>T; or G>A is removed/filtered out after being selected as a pre-determined SNP but before a contamination probability is determined. In some embodiments, target SNP error rates are between 104 and 10-3. For example, FIG. 5A shows greater error rates (y-axis) for A>G; T>C; C>T; or G>A conversion types (x-axis) when analyzing SNPs from whole transcriptome data. In another example, FIG. 5B shows error rate (y-axis) for A>G; T>C; C>T; or G>A conversion types (x-axis) when analyzing SNPs from targeted panels.

In some embodiments, the steps of selecting one or more pre-determined SNPs to be included in the contamination detection method includes determining whether the one or more pre-determined SNPs enable a contamination limit of detection (LoD) approaching the assay error rate. In some embodiments, the assay error rate is between about 104 to about 10-3 (or any of the subranges therein). In some embodiments, the contamination LoD should be about 12/effective coverage (e.g., number of sequencing reads mapping to the genomic locations of the SNPs). In some embodiments, determining the contamination LoD includes determining how many one or more pre-determined SNPs are needed to detect contamination. Determining how many one or more pre-determined SNPs are needed to detect contamination can include, without limitation: determining LoD as =˜ 3/(0.5 (i.e., % of pre-determined SNPs that are homozygous SNPs)*0.5 (i.e., % of pre-determined SNPs that will have opposite haplotype in contaminating sample)*total sampling events); determining effective coverage as =number of SNPs*mean depth; determining LoD as =˜ 3/(0.25*effective coverage); and/or determining the number of SNPs=˜ 3/(0.25*LoD*mean_depth).

### III.A. Pre-Determined SNPs Including Universal Human Reference Alleles

In some embodiments, one or more pre-determined SNPs include an allele present in a universal human reference database. In some embodiments, a universal human reference includes a plurality of nucleic acid fragments isolated from common human cells lines. Non-limiting commercially available UHRs include: Agilent, Thermo Fisher, Stratagene, and Clontech. One or more of the exemplary UHRs described herein includes cell lines selected from: adenocarcinoma (e.g., mammary gland); melanoma; hepatoblastoma (e.g., liver); liposarcoma; adenocarcinoma (e.g., cervix); histiocytic lymphoma (e.g., macrophages and histocytes); embryonal carcinoma (e.g., testis); lymphoblastic leukemia (e.g., T lymphoblasts); glioblastoma (e.g., brain); plasmacytoma (e.g., myeloma and B-lymphocyte).

In one embodiment, an allele present in a UHR based is selected as a pre-determined SNP based, at least in part, on an allele frequency considered to be homozygous. For example, an allele present in a UHR is selected as a pre-determined SNP based, at least in part, on an allele frequency greater than 0.75 in a UHR. In some embodiments, an allele present in a UHR is selected as a pre-determined SNP based, at least in part, on the SNP having an allele frequency considered to be homozygous in a UHR and the SNP having an allele frequency considered not to be homozygous in a human sample (e.g., a previously sequenced human sample). For example, an allele present in a UHR is selected as a pre-determined SNP based, at least in part, on an allele frequency of at least 0.75 (e.g., a homozygous frequency) in a UHR and an allele frequency of 0.05 or less (e.g., a non-homozygous frequency) in a human sample.

In some embodiments, UHR allele frequencies are determined empirically by sequencing UHR samples and/or human plasma samples.

Non-limiting examples of one or more pre-determined SNPs having an allele present in a UHR are provided in Table 1.

TABLE 1
UHR Contamination SNPs.

| Chromosome | Position | Rs id | ref | alt |
|---|---|---|---|---|
| chr1 | 5986204 | rs12142270 | G | A |
| chr1 | 6523171 | rs79620905 | G | A |
| chr1 | 10458539 | rs3927586 | C | T |
| chr1 | 10460323 | rs189080634 | C | T |
| chr1 | 12511291 | rs188379454 | C | T |
| chr1 | 13823620 | rs12091217 | C | T |
| chr1 | 13823643 | rs3820012 | C | T |
| chr1 | 16972632 | rs74058349 | C | T |
| chr1 | 16972633 | rs57600976 | A | G |
| chr1 | 23086965 | rs580878 | T | G |
| chr1 | 23344310 | rs12409193 | G | C |
| chr1 | 23360284 | rs17437528 | C | T |
| chr1 | 23967759 | rs4276860 | C | T |
| chr1 | 26278031 | rs75267699 | C | A |
| chr1 | 26787988 | rs113400508 | A | C |
| chr1 | 26877237 | rs34696599 | A | T |
| chr1 | 27760946 | rs74422309 | G | T |
| chr1 | 28497374 | rs58666060 | G | A |
| chr1 | 32683334 | rs16835131 | G | A |
| chr1 | 34850757 | rs12408762 | C | T |
| chr1 | 53767838 | rs71637818 | T | C |
| chr1 | 63555425 | rs2273367 | G | A |
| chr1 | 67002507 | rs11208986 | T | C |
| chr1 | 76700308 | rs74089738 | G | C |
| chr1 | 77947094 | rs17382996 | C | A |
| chr1 | 78016475 | rs114634955 | G | T |
| chr1 | 88980723 | rs79207870 | T | C |
| chr1 | 120459512 | rs587741250 | A | G |
| chr1 | 147156795 | rs17159890 | A | C |
| chr1 | 150476231 | rs12141218 | T | C |
| chr1 | 150476290 | rs1043293 | G | C |
| chr1 | 151118900 | rs76044622 | G | A |
| chr1 | 154270981 | rs12354278 | A | T |
| chr1 | 155336404 | rs114130331 | T | C |
| chr1 | 155336406 | rs41264227 | C | T |
| chr1 | 159781012 | rs3806189 | G | C |
| chr1 | 165910654 | rs3748701 | A | G |
| chr1 | 165910794 | rs512542 | A | G |
| chr1 | 166852396 | rs2232521 | C | T |
| chr1 | 179114945 | rs2274230 | T | G |
| chr1 | 179126413 | rs28914528 | C | T |
| chr1 | 179357215 | rs41308413 | T | C |
| chr1 | 205145282 | rs116436604 | T | C |
| chr1 | 207077173 | rs191886349 | A | T |
| chr1 | 228178017 | rs74142627 | G | A |
| chr1 | 234465684 | rs10910439 | C | T |
| chr1 | 234467544 | rs17378453 | C | T |
| chr2 | 26385124 | rs934280 | T | C |
| chr2 | 32310165 | rs78717808 | C | T |
| chr2 | 37672495 | rs17552689 | G | T |
| chr2 | 37929302 | rs61743792 | T | C |
| chr2 | 38295800 | rs114095450 | A | G |
| chr2 | 43291704 | rs17030648 | A | G |
| chr2 | 46617213 | rs77297964 | T | C |
| chr2 | 47153006 | rs17036300 | T | C |
| chr2 | 58046916 | rs377653814 | T | G |
| chr2 | 69324945 | rs73937246 | C | A |
| chr2 | 72178536 | rs17007922 | A | G |
| chr2 | 86042040 | rs34892520 | C | T |
| chr2 | 86045635 | rs1561328 | G | A |
| chr2 | 127845452 | rs71420810 | C | T |
| chr2 | 151481889 | rs148318449 | C | G |
| chr2 | 169639299 | rs117408837 | T | A |
| chr2 | 169639505 | rs1345141 | C | T |
| chr2 | 170082054 | rs17635525 | T | C |
| chr2 | 173226200 | rs60607753 | G | C |
| chr2 | 190502110 | rs116319890 | A | G |
| chr2 | 198147193 | rs150952998 | C | T |
| chr2 | 210022037 | rs59166419 | G | A |
| chr2 | 218663143 | rs35843327 | T | C |
| chr2 | 227560014 | rs6706723 | C | T |
| chr2 | 238245808 | rs28391755 | G | A |
| chr2 | 238399334 | rs4663891 | G | A |
| chr2 | 240560414 | rs55672855 | A | T |
| chr3 | 33147222 | rs11925558 | C | T |
| chr3 | 42552527 | rs663258 | C | T |
| chr3 | 44443735 | rs6790563 | A | G |
| chr3 | 44659626 | rs116792244 | C | T |
| chr3 | 49720391 | rs115380029 | G | A |
| chr3 | 111962298 | rs712520 | A | T |
| chr3 | 113366296 | rs74521061 | T | C |
| chr3 | 121663333 | rs2055034 | A | G |
| chr3 | 155937990 | rs113093609 | T | C |
| chr3 | 155941353 | rs146004589 | G | A |
| chr3 | 179393702 | rs6807219 | C | A |
| chr3 | 197671475 | rs73891683 | T | G |
| chr4 | 1979994 | rs111668967 | A | T |
| chr4 | 2231282 | rs3762942 | G | A |
| chr4 | 3240931 | rs73792381 | C | T |
| chr4 | 8441314 | rs3806811 | C | T |
| chr4 | 8452019 | rs61738667 | A | G |
| chr4 | 8471112 | rs17202499 | C | T |
| chr4 | 90309428 | rs12647859 | G | A |
| chr4 | 119512860 | rs61747388 | G | A |
| chr4 | 158667824 | rs11544037 | A | C |
| chr4 | 158905715 | rs191078590 | C | A |
| chr4 | 183271125 | rs11734376 | G | T |
| chr5 | 34955139 | rs12163995 | A | T |
| chr5 | 40828376 | rs389737 | T | C |
| chr5 | 43044751 | rs77862184 | G | A |
| chr5 | 43175771 | rs72752507 | T | C |
| chr5 | 56921369 | rs3756586 | A | G |
| chr5 | 79325900 | rs58646908 | G | C |
| chr5 | 79976898 | rs16877381 | T | C |
| chr5 | 151491719 | rs14160 | T | C |
| chr5 | 178228511 | rs11740356 | T | G |
| chr5 | 178867059 | rs11955074 | G | A |
| chr5 | 180847654 | rs17080695 | G | A |
| chr6 | 7249227 | rs78588343 | G | A |
| chr6 | 11135128 | rs61744084 | C | T |
| chr6 | 26523531 | rs11962165 | C | A |
| chr6 | 28359594 | rs733743 | G | C |
| chr6 | 31952179 | rs760070 | T | C |
| chr6 | 33457224 | rs114055571 | C | A |
| chr6 | 39109465 | rs78552786 | C | T |
| chr6 | 41787527 | rs115742810 | T | C |
| chr6 | 42880985 | rs78833648 | G | C |
| chr6 | 43337060 | rs74725336 | T | C |
| chr6 | 43523071 | rs7755135 | C | T |
| chr6 | 43523597 | rs55671916 | T | C |
| chr6 | 52498067 | rs7746960 | A | T |
| chr6 | 52502086 | rs9474230 | G | A |
| chr6 | 70526513 | rs7740873 | C | T |
| chr6 | 89643143 | rs7682 | G | A |
| chr6 | 89661483 | rs9444701 | G | A |
| chr6 | 89745365 | rs9359861 | A | G |
| chr6 | 89789783 | rs1036853 | G | A |
| chr6 | 100642669 | rs7755630 | T | A |
| chr6 | 109633049 | rs1406957 | C | T |
| chr6 | 111299555 | rs465646 | G | A |
| chr6 | 136792464 | rs140110518 | T | C |
| chr6 | 145954847 | rs117586623 | T | G |
| chr6 | 158509260 | rs192341971 | A | T |
| chr7 | 5306878 | rs182445426 | A | T |
| chr7 | 7567093 | rs6973400 | T | C |
| chr7 | 23174333 | rs2286273 | A | G |
| chr7 | 40095565 | rs17538342 | C | T |
| chr7 | 70792611 | rs56026275 | C | T |
| chr7 | 101238809 | rs7808669 | G | A |
| chr7 | 128305115 | rs6467170 | T | C |
| chr7 | 134291597 | rs61739885 | G | A |
| chr7 | 135361800 | rs1003226 | C | T |
| chr7 | 149284204 | rs11980276 | C | T |
| chr7 | 155780606 | rs62482831 | C | A |
| chr8 | 6643551 | rs116253794 | T | C |
| chr8 | 11324946 | rs7016671 | A | G |
| chr8 | 11327381 | rs2572402 | C | G |
| chr8 | 11327428 | rs3174048 | G | A |
| chr8 | 28093153 | rs2305451 | C | T |
| chr8 | 31167122 | rs1801196 | C | T |
| chr8 | 42169347 | rs72641449 | G | A |
| chr8 | 42171057 | rs114394395 | G | A |
| chr8 | 65709176 | rs76100380 | G | A |
| chr8 | 65709330 | rs80330597 | A | G |
| chr8 | 80520570 | rs78450036 | G | A |
| chr8 | 130016625 | rs185031455 | C | T |
| chr8 | 142271417 | rs34469664 | C | G |
| chr8 | 142664564 | rs35419434 | G | A |
| chr8 | 144520715 | rs79312814 | C | T |
| chr8 | 144523760 | rs11996936 | C | T |
| chr8 | 144804213 | rs2979086 | C | T |
| chr8 | 144807329 | rs10093836 | A | T |
| chr9 | 2043547 | rs76584435 | G | T |
| chr9 | 37441653 | rs17502738 | T | C |
| chr9 | 77416948 | rs1048743 | C | T |
| chr9 | 92614823 | rs3802383 | G | A |
| chr9 | 92642766 | rs35248147 | A | C |
| chr9 | 104134528 | rs7872034 | G | A |
| chr9 | 111649611 | rs1322259 | C | T |
| chr9 | 124878759 | rs2781055 | T | C |
| chr9 | 126506664 | rs113181570 | G | C |
| chr9 | 132905818 | rs118203576 | T | C |
| chr9 | 136428749 | rs1128877 | A | G |
| chr10 | 12121238 | rs111710934 | A | C |
| chr10 | 27093710 | rs79092403 | T | C |
| chr10 | 31807076 | rs10826997 | T | C |
| chr10 | 38120733 | rs71491238 | C | G |
| chr10 | 45000672 | rs12269028 | A | T |
| chr10 | 48436427 | rs78986194 | C | T |
| chr10 | 48439026 | rs115095528 | C | G |
| chr10 | 49470783 | rs4253207 | A | G |
| chr10 | 50625153 | rs74131448 | A | G |
| chr10 | 68482960 | rs3200066 | A | G |
| chr10 | 78013656 | rs12255950 | C | A |
| chr10 | 99696057 | rs61744356 | C | T |
| chr10 | 101556894 | rs11595968 | A | G |
| chr10 | 113911054 | rs17775775 | T | C |
| chr10 | 113914404 | rs239855 | G | T |
| chr11 | 7998914 | rs75048892 | C | T |
| chr11 | 57528575 | rs113266452 | C | A |
| chr11 | 62152097 | rs117392689 | G | C |
| chr11 | 62751391 | rs7945873 | C | T |
| chr11 | 72292875 | rs146071204 | C | A |
| chr11 | 85659899 | rs3168151 | C | G |
| chr11 | 94873768 | rs73520328 | C | T |
| chr11 | 117412910 | rs572884 | A | G |
| chr11 | 117412918 | rs572862 | A | G |
| chr12 | 276657 | rs74055605 | C | T |
| chr12 | 48935912 | rs2272311 | A | G |
| chr12 | 50176736 | rs9364 | G | A |
| chr12 | 55729581 | rs2231462 | G | A |
| chr12 | 69579004 | rs61759450 | G | A |
| chr12 | 89522129 | rs73194597 | G | A |
| chr12 | 89523034 | rs2230283 | C | T |
| chr12 | 95217374 | rs79350049 | C | A |
| chr12 | 95514973 | rs1057739 | C | T |
| chr12 | 98603278 | rs12579609 | A | G |
| chr12 | 98603497 | rs73372793 | C | T |
| chr12 | 107713138 | rs9302 | T | C |
| chr12 | 109081384 | rs78885554 | C | T |
| chr12 | 120461188 | rs111706861 | T | C |
| chr12 | 120461202 | rs141193769 | C | T |
| chr12 | 125102732 | rs3763984 | G | A |
| chr12 | 130790699 | rs73457930 | G | A |
| chr12 | 132677409 | rs5744751 | G | A |
| chr13 | 19824602 | rs9508908 | C | T |
| chr13 | 19864053 | rs374181504 | G | A |
| chr13 | 20086976 | rs259778 | A | G |
| chr13 | 20086978 | rs17076304 | G | A |
| chr13 | 23355916 | rs2031640 | A | T |
| chr13 | 27547151 | rs41291674 | G | A |
| chr13 | 41692954 | rs61752294 | A | G |
| chr13 | 52032939 | rs17480469 | A | G |
| chr13 | 52156124 | rs17482764 | T | A |
| chr13 | 52690781 | rs60220067 | A | G |
| chr13 | 52691063 | rs55875061 | G | A |
| chr13 | 52691209 | rs114906892 | C | T |
| chr13 | 52698713 | rs7994615 | G | A |
| chr13 | 52699435 | rs4261418 | C | T |
| chr13 | 52700492 | rs893070 | T | C |
| chr13 | 98023665 | rs78905111 | T | G |
| chr13 | 98023697 | rs17190392 | A | G |
| chr14 | 20287631 | rs61995495 | A | G |
| chr14 | 20287647 | rs112746533 | G | A |
| chr14 | 24308385 | rs2180197 | C | G |
| chr14 | 31095061 | rs111287623 | G | A |
| chr14 | 60091966 | rs160239 | T | C |
| chr14 | 67122363 | rs77465022 | T | C |
| chr14 | 67333008 | rs72717392 | A | G |
| chr14 | 67334999 | rs1044750 | T | C |
| chr14 | 76210098 | rs17104259 | T | C |
| chr14 | 90286410 | rs116980182 | G | A |
| chr14 | 90288582 | rs116195915 | A | C |
| chr14 | 90301263 | rs3825661 | C | T |
| chr14 | 96317747 | rs116026484 | A | G |
| chr15 | 28654355 | rs12898266 | T | C |
| chr15 | 28654366 | rs191045372 | G | A |
| chr15 | 28654369 | rs7173744 | G | A |
| chr15 | 28684798 | rs366916 | C | T |
| chr15 | 30942802 | rs3512 | G | C |
| chr15 | 42351331 | rs7181742 | T | C |
| chr15 | 42543195 | rs115365491 | A | T |
| chr15 | 42739217 | rs116819722 | C | T |
| chr15 | 44534882 | rs76263379 | C | T |
| chr15 | 64138408 | rs749504 | T | C |
| chr15 | 78157089 | rs62009337 | A | G |
| chr15 | 84622201 | rs114072014 | G | C |
| chr15 | 84632227 | rs16974462 | C | A |
| chr15 | 89295005 | rs7183618 | A | G |
| chr15 | 89295087 | rs35875311 | A | T |
| chr15 | 89315311 | rs34557339 | C | T |
| chr15 | 101654200 | rs520897 | T | C |
| chr16 | 1364674 | rs58261732 | G | T |
| chr16 | 1510110 | rs9454 | C | T |
| chr16 | 1655954 | rs77482527 | C | T |
| chr16 | 1675036 | rs73499799 | C | T |
| chr16 | 1676950 | rs7186654 | A | G |
| chr16 | 2501014 | rs76267944 | C | T |
| chr16 | 2528606 | rs139057608 | G | C |
| chr16 | 3656696 | rs8176919 | G | A |
| chr16 | 4351289 | rs569946035 | G | T |
| chr16 | 8868261 | rs75598828 | A | T |
| chr16 | 11180222 | rs11554587 | C | T |
| chr16 | 13937838 | rs2020958 | A | G |
| chr16 | 19552615 | rs116094698 | T | C |
| chr16 | 27648710 | rs61738361 | A | G |
| chr16 | 31457117 | rs28533031 | A | C |
| chr16 | 57178738 | rs767505 | A | G |
| chr16 | 69323361 | rs55955633 | G | A |
| chr16 | 69326884 | rs116676358 | G | A |
| chr16 | 74999399 | rs8053898 | C | T |
| chr16 | 80601103 | rs4281727 | C | T |
| chr16 | 88672051 | rs115005210 | C | T |
| chr16 | 88672063 | rs114081068 | C | T |
| chr17 | 1712461 | rs61736712 | C | T |
| chr17 | 2380005 | rs66647248 | A | G |
| chr17 | 3609443 | rs1977021 | G | A |
| chr17 | 6578999 | rs1063090 | A | T |
| chr17 | 6612072 | rs79173884 | T | G |
| chr17 | 6620978 | rs9889363 | T | A |
| chr17 | 8370336 | rs74532943 | G | A |
| chr17 | 17166232 | rs3744129 | C | T |
| chr17 | 30632161 | rs383436 | A | G |
| chr17 | 35118530 | rs9901455 | G | A |
| chr17 | 40089538 | rs12939700 | C | A |
| chr17 | 42573361 | rs2292754 | A | T |
| chr17 | 45061041 | rs115000396 | G | T |
| chr17 | 47050397 | rs199631359 | G | A |
| chr17 | 64129078 | rs3088093 | G | A |
| chr17 | 68131640 | rs112960508 | C | T |
| chr17 | 74864531 | rs34038065 | G | A |
| chr17 | 75629206 | rs820190 | G | A |
| chr17 | 79083494 | rs61756761 | A | G |
| chr17 | 81196776 | rs1542961 | C | T |
| chr17 | 81198167 | rs2659016 | A | G |
| chr18 | 13665767 | rs55800471 | A | G |
| chr18 | 36177087 | rs627107 | G | A |
| chr18 | 36177397 | rs72888759 | C | G |
| chr18 | 45879182 | rs34545102 | A | G |
| chr18 | 54361799 | rs1657907 | G | C |
| chr18 | 57027877 | rs187140119 | T | G |
| chr18 | 74158726 | rs17088882 | A | G |
| chr18 | 74632282 | rs17817969 | C | T |
| chr18 | 74633934 | rs948615 | A | C |
| chr18 | 74634538 | rs3764505 | C | G |
| chr18 | 75198514 | rs149526382 | C | A |
| chr19 | 2428255 | rs1050009 | A | G |
| chr19 | 3537186 | rs77733715 | A | G |
| chr19 | 4683280 | rs10404657 | G | A |
| chr19 | 4867678 | rs262559 | A | G |
| chr19 | 5910179 | rs73539613 | T | C |
| chr19 | 9527550 | rs73002164 | G | A |
| chr19 | 10112186 | rs112647895 | G | A |
| chr19 | 11780091 | rs35459645 | A | G |
| chr19 | 11832737 | rs117998813 | G | A |
| chr19 | 11903924 | rs141687609 | G | A |
| chr19 | 11948728 | rs111342482 | G | A |
| chr19 | 12076042 | rs6511763 | G | C |
| chr19 | 12156716 | rs269824 | T | C |
| chr19 | 12333574 | rs61744368 | G | A |
| chr19 | 12629947 | rs116279746 | T | C |
| chr19 | 16646165 | rs10411230 | G | A |
| chr19 | 18364168 | rs34177209 | T | A |
| chr19 | 18669828 | rs76401518 | G | A |
| chr19 | 18670107 | rs3795028 | G | A |
| chr19 | 20553833 | rs111988999 | C | T |
| chr19 | 32385828 | rs371145688 | A | C |
| chr19 | 34355051 | rs10415052 | A | G |
| chr19 | 39412913 | rs114784999 | T | C |
| chr19 | 43596055 | rs76868266 | G | C |
| chr19 | 45145850 | rs564069481 | A | C |
| chr19 | 45549636 | rs79660166 | T | C |
| chr19 | 52067461 | rs16983412 | C | G |
| chr19 | 52556065 | rs111288576 | C | T |
| chr19 | 52556292 | rs73578236 | C | T |
| chr19 | 56404363 | rs367599155 | C | G |
| chr19 | 57220592 | rs78525853 | G | A |
| chr19 | 57254933 | rs74851517 | G | A |
| chr19 | 57307340 | rs61997216 | A | G |
| chr19 | 57420659 | rs2158009 | C | T |
| chr19 | 57844874 | rs74643639 | A | G |
| chr19 | 57845421 | rs75849016 | G | C |
| chr19 | 57907991 | rs117176080 | T | A |
| chr19 | 58127929 | rs34445868 | G | A |
| chr19 | 58128960 | rs34255209 | T | C |
| chr19 | 58471080 | rs61742224 | A | G |
| chr20 | 277092 | rs2277781 | A | G |
| chr20 | 328519 | rs537465605 | T | C |
| chr20 | 18315086 | rs34099160 | C | T |
| chr20 | 18315829 | rs1050475 | C | T |
| chr20 | 25615010 | rs117999895 | T | G |
| chr20 | 35467383 | rs115994448 | G | A |
| chr20 | 39018823 | rs36025205 | C | T |
| chr20 | 39038539 | rs3752302 | C | T |
| chr20 | 62390662 | rs41312298 | T | C |
| chr21 | 14962939 | rs59988518 | C | T |
| chr21 | 33838178 | rs1802359 | C | T |
| chr21 | 39195426 | rs2836936 | G | A |
| chr21 | 43031766 | rs77084451 | G | A |
| chr21 | 44329821 | rs73907170 | T | C |
| chr21 | 46411395 | rs58559714 | G | A |
| chr21 | 46416292 | rs35978208 | A | C |
| chr21 | 46416302 | rs60444527 | A | G |
| chr21 | 46416481 | rs1044998 | T | G |
| chr21 | 46436996 | rs60078675 | C | T |
| chr22 | 18091949 | rs362128 | C | T |
| chr22 | 19847021 | rs60170553 | G | A |
| chr22 | 21484012 | rs199663506 | C | T |
| chr22 | 29507128 | rs6006177 | T | C |
| chr22 | 31906744 | rs5998170 | C | T |
| chr22 | 41688998 | rs73161345 | A | C |
| chr22 | 46237654 | rs115356860 | C | T |
| chr22 | 46239779 | rs73886769 | G | A |
| chr22 | 46241548 | rs11538240 | A | G |
| chr22 | 46242773 | rs73177043 | C | A |

### III.B. Pre-Determined Snps Including Ncbi Dbsnp Alleles

In some embodiments, one or more pre-determined SNPs include an allele present in a National Center for Biotechnology Information's (NCBI) Single Nucleotide Database (“dbSNP”) (e.g., dbSNP Build 155). The NCBI dbSNP database includes greater than 500 million SNPs compiled from various sources, which are vetted by NCBI before being placed into the dbSNP.

In some embodiments, an allele present in the NCBI dbSNP database is selected as a pre-determined SNP based, at least in part, on having a reference allele frequency in a range between 0.2 and 0.8. In some embodiments, an allele present in the NCBI dbSNP database is selected as a pre-determined SNP based, at least in part, on having a reference allele frequency between 0.3 and 0.7. In some embodiments, an allele present in the NCBI dbSNP database is selected as a pre-determined SNP based, at least in part, on having a reference allele frequency between 0.4 and 0.6.

In some embodiments, an allele present in the NCBI dbSNP database is selected as a pre-determined SNP based, at least in part, on allele frequency comprising a MAF, a VAF, sequencing depth, or any combination thereof. For example, an allele present in the NCBI dbSNP database is selected as a pre-determine SNP based, at least in part, on having a MAF in a range between 0.3 and 0.7, or optionally in a range between 0.4 and 0.6.

In some embodiments, one or more pre-determined SNPs that are present in the dbSNP database are not used as a pre-determined SNP because the SNP is a conversion type comprising: A>G; T>C; C>T; or G>A (See, e.g., FIGS. 5A-5B). In some cases, these types of conversions can be difficult to differentiate from low-level contamination events and so SNPs that match these conversion types can be excluded. In some embodiments, a pre-determined SNPs present in the dbSNP database having a conversion type comprising A>G; T>C; C>T; or G>A is removed/filtered out after being selected as a pre-determined SNP but before a contamination probability is determined.

Non-limiting examples of a pre-determined SNP having an allele present in the dbSNP database where the allele has a reference allele frequency in a range between 0.3 and 0.7 are provided in Table 2.

TABLE 2
CfRNA Contamination SNPs

| Chromosome | Position | Rs id | ref | alt |
|---|---|---|---|---|
| chr1 | 852019 | rs2905055 | G | T |
| chr1 | 1732412 | rs2294486 | G | C |
| chr1 | 1737504 | rs28537345 | A | C |
| chr1 | 1751981 | rs8841 | A | T |
| chr1 | 2556224 | rs2227312 | C | A |
| chr1 | 2581616 | rs4486391 | A | T |
| chr1 | 3780326 | rs8379 | A | C |
| chr1 | 3836572 | rs2275824 | A | T |
| chr1 | 3857169 | rs13374773 | C | A |
| chr1 | 6393650 | rs58110988 | T | G |
| chr1 | 9267328 | rs1294015 | T | G |
| chr1 | 9267890 | rs12314 | A | C |
| chr1 | 9368626 | rs9442601 | T | G |
| chr1 | 9850299 | rs935072 | A | T |
| chr1 | 15583355 | rs6429757 | C | G |
| chr1 | 15662646 | rs7536654 | C | G |
| chr1 | 15664488 | rs17448966 | T | G |
| chr1 | 17067553 | rs35058101 | T | A |
| chr1 | 17086626 | rs2076615 | A | C |
| chr1 | 19121349 | rs1044010 | C | G |
| chr1 | 19238850 | rs709683 | C | G |
| chr1 | 19682387 | rs9064 | G | T |
| chr1 | 19771448 | rs10917536 | G | T |
| chr1 | 21345450 | rs2072654 | T | G |
| chr1 | 21727934 | rs16825896 | C | A |
| chr1 | 22025547 | rs2255282 | G | T |
| chr1 | 22030736 | rs3820687 | A | T |
| chr1 | 22647804 | rs9434 | C | A |
| chr1 | 23092881 | rs3765407 | G | T |
| chr1 | 23520972 | rs2075995 | C | A |
| chr1 | 23871408 | rs2503000 | C | G |
| chr1 | 23872350 | rs6672157 | C | G |
| chr1 | 23872536 | rs2501423 | A | C |
| chr1 | 23872849 | rs2501425 | A | C |
| chr1 | 24156502 | rs7531447 | C | G |
| chr1 | 24536153 | rs196433 | T | G |
| chr1 | 25814082 | rs2294228 | C | A |
| chr1 | 27973568 | rs33981147 | T | A |
| chr1 | 37708513 | rs557897 | G | T |
| chr1 | 37708694 | rs7526362 | G | T |
| chr1 | 37862310 | rs3843 | G | T |
| chr1 | 39448691 | rs668556 | G | C |
| chr1 | 40509588 | rs4607875 | G | C |
| chr1 | 46027788 | rs1707336 | T | G |
| chr1 | 46055887 | rs785467 | A | T |
| chr1 | 46132597 | rs1707304 | C | A |
| chr1 | 46132601 | rs1707303 | A | C |
| chr1 | 47216345 | rs7664 | T | G |
| chr1 | 47217935 | rs2070929 | G | C |
| chr1 | 52826935 | rs475969 | T | A |
| chr1 | 53266643 | rs2297660 | G | T |
| chr1 | 54218183 | rs15921 | C | G |
| chr1 | 54716627 | rs1147990 | T | A |
| chr1 | 58655364 | rs10789069 | A | C |
| chr1 | 58655671 | rs232854 | T | A |
| chr1 | 58656617 | rs232852 | T | G |
| chr1 | 67409850 | rs4655708 | T | A |
| chr1 | 74206639 | rs489941 | C | A |
| chr1 | 74206956 | rs956 | T | A |
| chr1 | 74766547 | rs9647 | G | T |
| chr1 | 77564220 | rs1962523 | T | A |
| chr1 | 77713291 | rs6603958 | T | A |
| chr1 | 84205133 | rs1057738 | A | C |
| chr1 | 85250295 | rs12065422 | C | G |
| chr1 | 86351304 | rs272494 | T | A |
| chr1 | 88982295 | rs10754258 | T | A |
| chr1 | 89185167 | rs623134 | A | T |
| chr1 | 89186405 | rs1142889 | C | G |
| chr1 | 89633156 | rs10047070 | G | C |
| chr1 | 90020853 | rs2816881 | T | G |
| chr1 | 90032981 | rs954145 | G | T |
| chr1 | 93151846 | rs7532195 | T | G |
| chr1 | 93325880 | rs4847408 | G | C |
| chr1 | 93362966 | rs7525248 | T | A |
| chr1 | 93363691 | rs4847412 | C | G |
| chr1 | 99922947 | rs1804809 | A | C |
| chr1 | 100352622 | rs529224 | G | C |
| chr1 | 107765105 | rs7528153 | T | A |
| chr1 | 108937356 | rs168107 | G | T |
| chr1 | 111125554 | rs588885 | A | T |
| chr1 | 111197460 | rs600430 | T | G |
| chr1 | 111715923 | rs552802 | G | T |
| chr1 | 111725425 | rs197430 | G | C |
| chr1 | 112913924 | rs1049434 | A | T |
| chr1 | 114568062 | rs8128 | A | C |
| chr1 | 120451262 | rs77446849 | C | G |
| chr1 | 146065662 | rs199803686 | T | A |
| chr1 | 147225363 | rs2289575 | C | G |
| chr1 | 151695819 | rs1308137 | A | C |
| chr1 | 151760859 | rs8480 | T | G |
| chr1 | 151853515 | rs7556386 | G | T |
| chr1 | 153637410 | rs28510471 | C | G |
| chr1 | 155208991 | rs760077 | T | A |
| chr1 | 155247646 | rs116352080 | G | T |
| chr1 | 155247647 | rs115729781 | A | T |
| chr1 | 156211216 | rs2241108 | C | G |
| chr1 | 156464911 | rs1050316 | G | T |
| chr1 | 156915699 | rs4661012 | T | G |
| chr1 | 157677999 | rs11264794 | C | A |
| chr1 | 158636659 | rs3738791 | G | T |
| chr1 | 161226376 | rs3813628 | A | C |
| chr1 | 161631002 | rs76732376 | A | C |
| chr1 | 161631383 | rs34322334 | A | T |
| chr1 | 161727282 | rs72704099 | G | C |
| chr1 | 161961838 | rs2499849 | G | C |
| chr1 | 166851494 | rs3738209 | G | T |
| chr1 | 167420524 | rs2902147 | G | T |
| chr1 | 168244860 | rs10737541 | T | G |
| chr1 | 168246261 | rs2205699 | C | A |
| chr1 | 168251987 | rs12608 | A | C |
| chr1 | 168252748 | rs906 | G | T |
| chr1 | 169387595 | rs6427185 | G | T |
| chr1 | 169798939 | rs6668114 | C | A |
| chr1 | 171702323 | rs10798599 | T | G |
| chr1 | 173185165 | rs7514229 | G | T |
| chr1 | 173886160 | rs1322775 | A | T |
| chr1 | 173894430 | rs79526252 | A | T |
| chr1 | 173894431 | rs78007840 | T | A |
| chr1 | 179073315 | rs4652353 | T | G |
| chr1 | 179101199 | rs3813643 | C | G |
| chr1 | 180020607 | rs2477120 | G | C |
| chr1 | 182381761 | rs2296523 | C | G |
| chr1 | 182582202 | rs627928 | A | C |
| chr1 | 183926587 | rs4634865 | C | A |
| chr1 | 184691071 | rs1046239 | T | A |
| chr1 | 184694403 | rs9425343 | A | C |
| chr1 | 185118502 | rs12030554 | A | T |
| chr1 | 186421171 | rs8824 | A | C |
| chr1 | 201468349 | rs1256930 | A | T |
| chr1 | 203024070 | rs1046532 | C | A |
| chr1 | 204550059 | rs4252745 | C | G |
| chr1 | 204556440 | rs10900598 | G | T |
| chr1 | 205146022 | rs1061132 | C | A |
| chr1 | 205303855 | rs1106202 | C | G |
| chr1 | 206496836 | rs10836 | G | C |
| chr1 | 207715394 | rs7553211 | G | T |
| chr1 | 207881244 | rs1204679 | A | C |
| chr1 | 207883762 | rs1211538 | A | C |
| chr1 | 211571812 | rs11277 | C | G |
| chr1 | 214637901 | rs2070065 | C | G |
| chr1 | 222746553 | rs2378607 | T | G |
| chr1 | 224193219 | rs1060394 | A | T |
| chr1 | 226736237 | rs6667260 | A | C |
| chr1 | 229324499 | rs2282081 | A | T |
| chr1 | 229659323 | rs1048306 | T | G |
| chr1 | 230280653 | rs1043897 | G | T |
| chr1 | 230906986 | rs3811502 | T | A |
| chr1 | 236215849 | rs2449 | A | C |
| chr1 | 236217444 | rs2950396 | T | G |
| chr1 | 236218525 | rs1055851 | G | C |
| chr1 | 236249930 | rs2477599 | T | A |
| chr1 | 236548651 | rs1041942 | T | A |
| chr1 | 236548656 | rs1041943 | A | C |
| chr1 | 236895444 | rs12070777 | C | A |
| chr1 | 239714237 | rs6684622 | G | C |
| chr1 | 241630754 | rs3765820 | C | A |
| chr2 | 675831 | rs2293084 | G | T |
| chr2 | 3465692 | rs4971514 | G | C |
| chr2 | 3498284 | rs1130319 | T | G |
| chr2 | 3498427 | rs3349 | T | A |
| chr2 | 6896341 | rs7583850 | A | T |
| chr2 | 6896707 | rs6431838 | G | C |
| chr2 | 6896936 | rs6431839 | G | T |
| chr2 | 8297119 | rs3102945 | G | C |
| chr2 | 9388407 | rs2715860 | G | C |
| chr2 | 9489238 | rs13008101 | T | G |
| chr2 | 9919641 | rs1820965 | G | T |
| chr2 | 9936879 | rs4669504 | A | C |
| chr2 | 10448426 | rs28742580 | C | G |
| chr2 | 12741652 | rs1057001 | T | A |
| chr2 | 16551007 | rs4240234 | G | T |
| chr2 | 16551123 | rs4263114 | T | G |
| chr2 | 17665325 | rs2710674 | A | T |
| chr2 | 20685213 | rs9085 | A | C |
| chr2 | 25927720 | rs6738270 | G | T |
| chr2 | 25927904 | rs6728684 | T | G |
| chr2 | 25928774 | rs2072695 | A | T |
| chr2 | 27650459 | rs8731 | C | G |
| chr2 | 32488639 | rs2366894 | A | T |
| chr2 | 33564001 | rs8256 | C | G |
| chr2 | 37248833 | rs4670679 | G | C |
| chr2 | 37643365 | rs3731854 | C | G |
| chr2 | 38075034 | rs1056827 | C | A |
| chr2 | 38075247 | rs10012 | G | C |
| chr2 | 38295118 | rs6987 | A | T |
| chr2 | 38295501 | rs12712582 | G | T |
| chr2 | 38562366 | rs12329205 | T | A |
| chr2 | 42762854 | rs2278585 | G | T |
| chr2 | 42762961 | rs2278586 | G | C |
| chr2 | 46760796 | rs3768719 | T | G |
| chr2 | 48376344 | rs6705802 | A | C |
| chr2 | 48581743 | rs3749144 | T | A |
| chr2 | 48582454 | rs3792234 | G | T |
| chr2 | 53971158 | rs2949815 | G | T |
| chr2 | 55050505 | rs6545468 | C | G |
| chr2 | 55656182 | rs2627765 | G | T |
| chr2 | 64252707 | rs1963382 | G | C |
| chr2 | 64338094 | rs1426701 | G | T |
| chr2 | 68362190 | rs17035355 | C | A |
| chr2 | 68365183 | rs3732046 | A | C |
| chr2 | 69325389 | rs2667 | C | A |
| chr2 | 69431994 | rs4453725 | A | T |
| chr2 | 69462187 | rs60724200 | T | G |
| chr2 | 69881124 | rs1056482 | T | A |
| chr2 | 70447617 | rs503314 | G | C |
| chr2 | 70448449 | rs473698 | C | G |
| chr2 | 71130580 | rs981947 | G | C |
| chr2 | 71133014 | rs10199088 | A | C |
| chr2 | 71184199 | rs399251 | C | G |
| chr2 | 71611467 | rs2303606 | C | A |
| chr2 | 73700971 | rs2001490 | C | G |
| chr2 | 74215304 | rs828853 | T | G |
| chr2 | 74492783 | rs17009980 | G | T |
| chr2 | 74891203 | rs943 | T | G |
| chr2 | 75656264 | rs917236 | G | T |
| chr2 | 85319809 | rs4832164 | A | C |
| chr2 | 86841659 | rs15800 | T | G |
| chr2 | 96251850 | rs7058 | T | G |
| chr2 | 99376359 | rs7558074 | A | C |
| chr2 | 99549992 | rs13427251 | A | T |
| chr2 | 102356339 | rs4851566 | G | C |
| chr2 | 102716131 | rs1051783 | T | G |
| chr2 | 108508103 | rs2378155 | C | A |
| chr2 | 108812690 | rs975597 | T | A |
| chr2 | 112334108 | rs6761599 | T | G |
| chr2 | 112334856 | rs7557862 | C | A |
| chr2 | 112550939 | rs2304555 | T | A |
| chr2 | 113612069 | rs1665293 | C | A |
| chr2 | 113756521 | rs7592689 | A | C |
| chr2 | 118013990 | rs11545372 | C | A |
| chr2 | 119980249 | rs1046433 | C | A |
| chr2 | 120013132 | rs2276586 | A | C |
| chr2 | 127701640 | rs10206957 | C | G |
| chr2 | 130152246 | rs3192417 | G | C |
| chr2 | 130152309 | rs3192414 | C | G |
| chr2 | 131498932 | rs3817572 | A | C |
| chr2 | 134453973 | rs1041938 | A | T |
| chr2 | 135985573 | rs2278682 | G | C |
| chr2 | 144141992 | rs3731958 | C | A |
| chr2 | 149587175 | rs4667420 | C | G |
| chr2 | 151248577 | rs34132424 | C | A |
| chr2 | 151476790 | rs13555 | C | A |
| chr2 | 159616888 | rs1046496 | A | T |
| chr2 | 161308497 | rs9713 | A | T |
| chr2 | 165748500 | rs13429321 | A | T |
| chr2 | 169636593 | rs1050354 | T | A |
| chr2 | 171556106 | rs7585194 | C | A |
| chr2 | 175927031 | rs7571968 | A | C |
| chr2 | 178504189 | rs3731754 | C | G |
| chr2 | 179106363 | rs2008989 | T | G |
| chr2 | 179264718 | rs12693183 | G | T |
| chr2 | 182757820 | rs288334 | T | G |
| chr2 | 182779178 | rs288241 | A | T |
| chr2 | 183098602 | rs2138485 | C | A |
| chr2 | 184598458 | rs359895 | T | A |
| chr2 | 187466457 | rs13392310 | A | T |
| chr2 | 190204963 | rs11542 | T | A |
| chr2 | 196197069 | rs12472336 | A | T |
| chr2 | 200490212 | rs3795969 | C | G |
| chr2 | 201217736 | rs13006529 | T | A |
| chr2 | 201287439 | rs13113 | T | A |
| chr2 | 207825736 | rs2306432 | G | T |
| chr2 | 217799910 | rs3747 | T | G |
| chr2 | 217800324 | rs9579 | T | G |
| chr2 | 218217396 | rs2271541 | G | T |
| chr2 | 218568230 | rs500317 | G | C |
| chr2 | 218568272 | rs500422 | C | A |
| chr2 | 218568634 | rs524902 | A | C |
| chr2 | 218658710 | rs4674324 | T | G |
| chr2 | 218737776 | rs3731877 | G | C |
| chr2 | 227357577 | rs8222 | C | G |
| chr2 | 227559055 | rs4312485 | C | G |
| chr2 | 229024552 | rs3755302 | A | T |
| chr2 | 230168267 | rs4973282 | C | A |
| chr2 | 230168572 | rs7583955 | A | C |
| chr2 | 231524818 | rs3752760 | C | G |
| chr2 | 232735194 | rs11555646 | A | C |
| chr2 | 234494012 | rs10194289 | G | T |
| chr2 | 236124429 | rs1530936 | T | G |
| chr2 | 238098522 | rs73098352 | C | A |
| chr2 | 238099209 | rs1054641 | T | A |
| chr2 | 239048664 | rs895808 | A | C |
| chr2 | 241095731 | rs2240538 | G | T |
| chr2 | 241143065 | rs758068 | A | C |
| chr3 | 3782652 | rs769639 | C | G |
| chr3 | 4361469 | rs14275 | T | G |
| chr3 | 4675127 | rs2306877 | A | C |
| chr3 | 9757089 | rs1052133 | C | G |
| chr3 | 11555613 | rs4684789 | G | T |
| chr3 | 11846785 | rs420599 | C | G |
| chr3 | 14145949 | rs2228001 | G | T |
| chr3 | 14671551 | rs11717438 | G | T |
| chr3 | 14671614 | rs11717411 | C | G |
| chr3 | 14897972 | rs2164356 | G | T |
| chr3 | 16264167 | rs14080 | C | G |
| chr3 | 16286348 | rs842274 | T | G |
| chr3 | 16316471 | rs842424 | T | A |
| chr3 | 27716784 | rs2887944 | G | T |
| chr3 | 28478926 | rs1563656 | T | A |
| chr3 | 31991251 | rs13094125 | T | G |
| chr3 | 32166245 | rs6799728 | A | T |
| chr3 | 33439908 | rs2272153 | G | C |
| chr3 | 33867556 | rs7651053 | G | C |
| chr3 | 36988684 | rs9311149 | C | A |
| chr3 | 39281672 | rs11715522 | A | C |
| chr3 | 40451727 | rs6801859 | G | T |
| chr3 | 40464175 | rs13095055 | G | T |
| chr3 | 42225341 | rs9156 | C | A |
| chr3 | 45594721 | rs267239 | C | G |
| chr3 | 46408487 | rs11266744 | A | C |
| chr3 | 46408579 | rs3204849 | T | A |
| chr3 | 47347457 | rs8180040 | T | A |
| chr3 | 47851089 | rs1061003 | G | C |
| chr3 | 48440024 | rs9876891 | T | G |
| chr3 | 52576635 | rs17264436 | T | A |
| chr3 | 52763618 | rs1029871 | G | C |
| chr3 | 56620806 | rs10865999 | C | G |
| chr3 | 57560266 | rs7618684 | A | C |
| chr3 | 58318881 | rs3210776 | C | G |
| chr3 | 58319508 | rs10687 | G | T |
| chr3 | 58565844 | rs1043956 | G | T |
| chr3 | 73067565 | rs7653851 | A | T |
| chr3 | 98580521 | rs1051712 | T | G |
| chr3 | 98793981 | rs14310 | T | A |
| chr3 | 100748832 | rs7297 | A | T |
| chr3 | 101347873 | rs2433031 | T | A |
| chr3 | 101782136 | rs2466368 | A | C |
| chr3 | 101826741 | rs622013 | A | T |
| chr3 | 101994628 | rs12629299 | C | A |
| chr3 | 112928985 | rs9826308 | A | C |
| chr3 | 112929280 | rs4596117 | T | G |
| chr3 | 113008337 | rs2306857 | A | T |
| chr3 | 114321119 | rs9879813 | T | G |
| chr3 | 119211944 | rs5868 | G | C |
| chr3 | 119823277 | rs60393216 | A | T |
| chr3 | 120394556 | rs1057231 | T | G |
| chr3 | 120395281 | rs13709 | A | C |
| chr3 | 120689323 | rs72625420 | A | C |
| chr3 | 122423056 | rs1962046 | C | G |
| chr3 | 122533357 | rs11921027 | T | G |
| chr3 | 122636889 | rs2650954 | C | G |
| chr3 | 122728727 | rs3732832 | A | C |
| chr3 | 123584974 | rs1271004 | G | C |
| chr3 | 124968923 | rs1909586 | G | T |
| chr3 | 128895342 | rs1680778 | A | C |
| chr3 | 129567570 | rs2245285 | G | C |
| chr3 | 131228591 | rs3738000 | A | T |
| chr3 | 133649518 | rs3192149 | T | G |
| chr3 | 134597864 | rs9857995 | G | C |
| chr3 | 138162423 | rs3732839 | T | A |
| chr3 | 142558733 | rs2227930 | A | T |
| chr3 | 143058666 | rs7623532 | C | A |
| chr3 | 143991426 | rs1979910 | A | C |
| chr3 | 152244427 | rs62272722 | A | T |
| chr3 | 153167810 | rs6785014 | A | T |
| chr3 | 154301098 | rs9438 | G | C |
| chr3 | 158672606 | rs9841 | A | T |
| chr3 | 158692158 | rs8650 | T | A |
| chr3 | 161075890 | rs12107243 | C | G |
| chr3 | 161078566 | rs1045448 | G | C |
| chr3 | 170085142 | rs1861935 | G | T |
| chr3 | 170089836 | rs6444896 | C | G |
| chr3 | 170090051 | rs6804888 | G | T |
| chr3 | 170396290 | rs1045210 | A | C |
| chr3 | 172397675 | rs6794474 | T | A |
| chr3 | 179234719 | rs9838117 | G | T |
| chr3 | 183452348 | rs10804889 | A | C |
| chr3 | 183490831 | rs2948135 | C | G |
| chr3 | 183680283 | rs10937148 | C | A |
| chr3 | 183682700 | rs11927407 | C | G |
| chr3 | 183684143 | rs11542855 | C | A |
| chr3 | 184711115 | rs9872799 | T | G |
| chr3 | 184711626 | rs10937187 | C | A |
| chr3 | 184915459 | rs4686879 | A | C |
| chr3 | 186147828 | rs2280210 | A | T |
| chr3 | 187371115 | rs1533595 | C | A |
| chr3 | 188877884 | rs1064607 | G | C |
| chr3 | 189147634 | rs2242013 | T | G |
| chr3 | 189150926 | rs1052437 | A | C |
| chr3 | 191396520 | rs2293378 | A | T |
| chr3 | 191397293 | rs4677732 | G | C |
| chr3 | 194590002 | rs1055161 | C | A |
| chr3 | 195277764 | rs7632534 | G | T |
| chr3 | 195910366 | rs56261799 | G | T |
| chr3 | 196235373 | rs870339 | G | T |
| chr3 | 196503693 | rs9837291 | G | C |
| chr3 | 196734603 | rs1047113 | A | C |
| chr3 | 197043111 | rs7641 | C | G |
| chr4 | 440673 | rs9328746 | A | T |
| chr4 | 766470 | rs7336 | G | T |
| chr4 | 959910 | rs4690326 | A | C |
| chr4 | 1170489 | rs2279279 | C | G |
| chr4 | 1717156 | rs2236787 | A | T |
| chr4 | 1745117 | rs8389 | A | T |
| chr4 | 2249484 | rs11649 | G | C |
| chr4 | 2834468 | rs73189445 | C | A |
| chr4 | 2836036 | rs1263416 | G | C |
| chr4 | 2837711 | rs735794 | G | C |
| chr4 | 3041786 | rs2857850 | A | C |
| chr4 | 6717048 | rs3172604 | G | T |
| chr4 | 7031197 | rs3756255 | A | T |
| chr4 | 16161642 | rs317854 | C | G |
| chr4 | 17486663 | rs699460 | T | G |
| chr4 | 17628569 | rs4698634 | G | T |
| chr4 | 17843615 | rs7688403 | G | C |
| chr4 | 36066949 | rs12645801 | A | T |
| chr4 | 38775552 | rs10856838 | A | T |
| chr4 | 38775615 | rs10856839 | T | G |
| chr4 | 38824455 | rs6822503 | C | A |
| chr4 | 38825193 | rs2381290 | T | A |
| chr4 | 39287688 | rs17754 | G | C |
| chr4 | 40244370 | rs1053509 | A | C |
| chr4 | 42020447 | rs15857 | C | A |
| chr4 | 42410670 | rs12639920 | T | G |
| chr4 | 44699747 | rs6817397 | T | G |
| chr4 | 47591266 | rs4145944 | G | C |
| chr4 | 48424049 | rs7664981 | A | T |
| chr4 | 51848345 | rs6851073 | C | G |
| chr4 | 56314450 | rs11723379 | G | C |
| chr4 | 67617773 | rs13348 | T | G |
| chr4 | 69727072 | rs2292092 | G | T |
| chr4 | 75528640 | rs9307834 | A | C |
| chr4 | 75917705 | rs7686066 | A | T |
| chr4 | 76021790 | rs3921 | C | G |
| chr4 | 76114975 | rs4730 | G | C |
| chr4 | 77031389 | rs17002335 | T | G |
| chr4 | 77169402 | rs11724432 | T | G |
| chr4 | 80072642 | rs13140055 | G | T |
| chr4 | 80203442 | rs12780 | G | C |
| chr4 | 82353055 | rs7691121 | C | G |
| chr4 | 83284719 | rs6818847 | C | A |
| chr4 | 83461399 | rs1126971 | A | T |
| chr4 | 84966274 | rs71597394 | C | G |
| chr4 | 86001034 | rs10305 | A | T |
| chr4 | 87138873 | rs342458 | C | A |
| chr4 | 87495036 | rs13051 | G | T |
| chr4 | 89243561 | rs756004 | C | G |
| chr4 | 89244491 | rs872614 | A | C |
| chr4 | 89244500 | rs872613 | T | A |
| chr4 | 89245232 | rs17015264 | A | C |
| chr4 | 89245627 | rs6532146 | C | A |
| chr4 | 89246223 | rs1431552 | G | T |
| chr4 | 89246225 | rs1431551 | A | T |
| chr4 | 89246355 | rs9790623 | G | C |
| chr4 | 89246446 | rs9790754 | T | G |
| chr4 | 89247264 | rs1431550 | A | T |
| chr4 | 98879023 | rs4699688 | G | C |
| chr4 | 102888488 | rs7254 | T | G |
| chr4 | 103025961 | rs17215211 | T | A |
| chr4 | 105708873 | rs3756260 | G | C |
| chr4 | 112277649 | rs701758 | G | C |
| chr4 | 112441466 | rs231253 | C | G |
| chr4 | 118710837 | rs1064034 | A | T |
| chr4 | 118715240 | rs298975 | G | T |
| chr4 | 121870446 | rs2271176 | G | C |
| chr4 | 123315824 | rs11930165 | C | G |
| chr4 | 142026393 | rs11100741 | C | G |
| chr4 | 143553513 | rs1391191 | A | C |
| chr4 | 146256316 | rs11930848 | T | G |
| chr4 | 153222954 | rs34449206 | C | G |
| chr4 | 153466445 | rs71620317 | G | C |
| chr4 | 158667824 | rs11544037 | A | C |
| chr4 | 163525131 | rs1053209 | T | A |
| chr4 | 165076223 | rs57550388 | T | G |
| chr4 | 165100659 | rs6536890 | G | C |
| chr4 | 184627976 | rs6948 | G | T |
| chr4 | 186211877 | rs1053094 | A | T |
| chr5 | 6633666 | rs248793 | C | G |
| chr5 | 10650212 | rs13354827 | T | G |
| chr5 | 10650213 | rs13354828 | T | G |
| chr5 | 31553161 | rs11748072 | T | G |
| chr5 | 32602840 | rs1046680 | T | A |
| chr5 | 34951045 | rs37439 | C | A |
| chr5 | 43015112 | rs160709 | A | C |
| chr5 | 43289606 | rs6814 | G | C |
| chr5 | 43526931 | rs4866747 | A | T |
| chr5 | 44819544 | rs9637783 | T | G |
| chr5 | 44826157 | rs7702464 | A | C |
| chr5 | 44827578 | rs6868232 | G | C |
| chr5 | 50843524 | rs27243 | A | T |
| chr5 | 62476708 | rs26635 | G | T |
| chr5 | 64719534 | rs898211 | G | C |
| chr5 | 68300033 | rs12755 | C | A |
| chr5 | 69123227 | rs164572 | A | T |
| chr5 | 69167187 | rs164390 | G | T |
| chr5 | 69217772 | rs2242350 | G | T |
| chr5 | 73580857 | rs13168040 | G | T |
| chr5 | 76969210 | rs1053989 | C | A |
| chr5 | 77431040 | rs335634 | A | C |
| chr5 | 78002795 | rs11552314 | A | T |
| chr5 | 78360288 | rs4530741 | A | C |
| chr5 | 78778375 | rs7704939 | A | C |
| chr5 | 78779095 | rs754566 | C | A |
| chr5 | 79325845 | rs3733886 | G | T |
| chr5 | 79685727 | rs3087813 | G | T |
| chr5 | 79978772 | rs10060444 | T | A |
| chr5 | 79981994 | rs6453495 | A | C |
| chr5 | 80141798 | rs10053887 | A | C |
| chr5 | 80142454 | rs12519111 | C | G |
| chr5 | 81417277 | rs11949697 | T | G |
| chr5 | 90516859 | rs3087840 | T | A |
| chr5 | 94707188 | rs7714195 | A | T |
| chr5 | 96783148 | rs27044 | G | C |
| chr5 | 97161619 | rs2216709 | A | C |
| chr5 | 98773712 | rs2545731 | T | G |
| chr5 | 100809684 | rs11584 | A | C |
| chr5 | 109337054 | rs33730 | T | A |
| chr5 | 110764969 | rs7376 | T | G |
| chr5 | 111489430 | rs31619 | G | T |
| chr5 | 112867867 | rs7213 | T | A |
| chr5 | 112869510 | rs439456 | G | C |
| chr5 | 113019201 | rs17372511 | C | G |
| chr5 | 113019971 | rs4778 | C | G |
| chr5 | 113553185 | rs72805422 | A | T |
| chr5 | 113593316 | rs1132528 | T | A |
| chr5 | 115208202 | rs10059069 | A | C |
| chr5 | 115522740 | rs12187973 | G | T |
| chr5 | 115615383 | rs698365 | T | G |
| chr5 | 115615443 | rs698366 | T | G |
| chr5 | 116092637 | rs1129494 | G | T |
| chr5 | 119355998 | rs3797339 | C | A |
| chr5 | 119395372 | rs1105769 | A | C |
| chr5 | 119395578 | rs1105771 | A | C |
| chr5 | 122775515 | rs1870560 | G | C |
| chr5 | 123614636 | rs3797534 | G | C |
| chr5 | 126626106 | rs1142104 | C | G |
| chr5 | 132482939 | rs6873426 | G | T |
| chr5 | 134899923 | rs319600 | A | C |
| chr5 | 136178801 | rs10038999 | T | A |
| chr5 | 136180287 | rs9327749 | T | G |
| chr5 | 136180500 | rs3206633 | T | G |
| chr5 | 138436607 | rs11334 | G | C |
| chr5 | 140332965 | rs7268 | A | C |
| chr5 | 140673766 | rs2530242 | G | C |
| chr5 | 148442572 | rs1128450 | T | G |
| chr5 | 148827884 | rs1042719 | G | C |
| chr5 | 149003782 | rs1432798 | C | G |
| chr5 | 149340524 | rs813035 | T | G |
| chr5 | 150527971 | rs2273235 | T | G |
| chr5 | 151661589 | rs3549 | G | C |
| chr5 | 154038399 | rs920310 | T | G |
| chr5 | 154458724 | rs734200 | A | C |
| chr5 | 157266552 | rs187458 | C | G |
| chr5 | 157269485 | rs767007 | C | G |
| chr5 | 160402051 | rs1128026 | A | T |
| chr5 | 169604214 | rs2042248 | G | T |
| chr5 | 170378625 | rs2656841 | T | G |
| chr5 | 170378626 | rs2656842 | G | T |
| chr5 | 175528569 | rs166641 | A | T |
| chr5 | 175529036 | rs156371 | G | T |
| chr5 | 177596097 | rs6634 | A | T |
| chr5 | 177632129 | rs6886539 | T | G |
| chr5 | 179623187 | rs1136267 | A | C |
| chr5 | 179863845 | rs30386 | T | G |
| chr5 | 180233786 | rs6703 | T | A |
| chr5 | 180235722 | rs4634313 | C | A |
| chr5 | 180810084 | rs936712 | G | C |
| chr6 | 711150 | rs2244443 | A | C |
| chr6 | 2855596 | rs375556 | G | C |
| chr6 | 2990660 | rs1054132 | G | T |
| chr6 | 3723530 | rs1045778 | G | T |
| chr6 | 3727577 | rs226959 | C | G |
| chr6 | 7862398 | rs17557 | G | C |
| chr6 | 8014471 | rs2748375 | A | C |
| chr6 | 13361695 | rs2496160 | G | T |
| chr6 | 13364317 | rs553948 | T | A |
| chr6 | 13790070 | rs3734669 | T | G |
| chr6 | 13790161 | rs3734668 | C | G |
| chr6 | 24533965 | rs1054899 | C | A |
| chr6 | 24804580 | rs11285 | G | C |
| chr6 | 26526713 | rs11754138 | G | C |
| chr6 | 26634838 | rs2259033 | G | C |
| chr6 | 27451068 | rs7509 | T | G |
| chr6 | 28363351 | rs13201753 | A | C |
| chr6 | 28380381 | rs1052215 | T | G |
| chr6 | 29723313 | rs1362125 | T | A |
| chr6 | 30283609 | rs1075105 | C | G |
| chr6 | 30287588 | rs1264623 | A | C |
| chr6 | 30292242 | rs1264619 | G | C |
| chr6 | 30908257 | rs2074510 | G | T |
| chr6 | 30909983 | rs1419693 | A | C |
| chr6 | 31202451 | rs9366770 | G | C |
| chr6 | 31394557 | rs1052405 | G | C |
| chr6 | 31400763 | rs2523452 | C | G |
| chr6 | 31477157 | rs2516435 | C | A |
| chr6 | 31477190 | rs2516515 | A | C |
| chr6 | 31533435 | rs11796 | A | T |
| chr6 | 31637671 | rs7889 | C | G |
| chr6 | 31795067 | rs2753960 | G | T |
| chr6 | 31896770 | rs7887 | G | T |
| chr6 | 32553965 | rs538116343 | A | T |
| chr6 | 32632812 | rs9272126 | G | C |
| chr6 | 32632824 | rs9272128 | C | A |
| chr6 | 32644028 | rs9273030 | T | A |
| chr6 | 32644097 | rs9273034 | G | T |
| chr6 | 32644532 | rs9273078 | T | A |
| chr6 | 32644779 | rs9273098 | C | G |
| chr6 | 32644871 | rs9273112 | A | T |
| chr6 | 32644887 | rs9273114 | C | G |
| chr6 | 32644895 | rs9273115 | C | A |
| chr6 | 32644922 | rs9273119 | T | A |
| chr6 | 32645023 | rs9273132 | T | G |
| chr6 | 32645979 | rs9273218 | C | A |
| chr6 | 32646160 | rs9273231 | C | A |
| chr6 | 32646167 | rs9273232 | A | T |
| chr6 | 32646180 | rs9273235 | T | A |
| chr6 | 32646196 | rs9273236 | G | C |
| chr6 | 32646605 | rs9273271 | A | C |
| chr6 | 32646637 | rs9273277 | T | A |
| chr6 | 32646734 | rs9273288 | T | A |
| chr6 | 32646928 | rs17843563 | T | G |
| chr6 | 32659473 | rs9273410 | C | A |
| chr6 | 33005736 | rs410168 | C | G |
| chr6 | 33067736 | rs1054031 | C | G |
| chr6 | 33083959 | rs542443316 | A | T |
| chr6 | 33086898 | rs9277529 | C | G |
| chr6 | 33691695 | rs2229642 | C | G |
| chr6 | 35295900 | rs8205 | T | A |
| chr6 | 35574699 | rs3800373 | C | A |
| chr6 | 36230800 | rs3748045 | G | C |
| chr6 | 36928661 | rs8472 | T | G |
| chr6 | 36954908 | rs1405069 | A | C |
| chr6 | 37028997 | rs708017 | C | G |
| chr6 | 37480218 | rs1874736 | C | G |
| chr6 | 41191484 | rs7754593 | G | T |
| chr6 | 41546673 | rs6935737 | C | G |
| chr6 | 41790098 | rs8393 | C | A |
| chr6 | 41921089 | rs2274578 | C | G |
| chr6 | 42082162 | rs6918636 | G | C |
| chr6 | 42206873 | rs8850 | G | T |
| chr6 | 43025087 | rs3749903 | C | G |
| chr6 | 43216394 | rs2273709 | A | C |
| chr6 | 43336269 | rs7692 | C | A |
| chr6 | 43523209 | rs11077 | T | G |
| chr6 | 43770613 | rs2010963 | C | G |
| chr6 | 45901893 | rs3224 | G | C |
| chr6 | 52498046 | rs1056709 | T | A |
| chr6 | 69697573 | rs12648 | A | T |
| chr6 | 71306704 | rs7753063 | C | A |
| chr6 | 75679273 | rs1018103 | T | G |
| chr6 | 75715878 | rs7385 | A | C |
| chr6 | 80344946 | rs1042367 | C | G |
| chr6 | 87512174 | rs1051148 | T | G |
| chr6 | 90515198 | rs157706 | A | T |
| chr6 | 98871364 | rs2743877 | T | A |
| chr6 | 99399384 | rs4144165 | G | T |
| chr6 | 106628908 | rs1987623 | A | T |
| chr6 | 107704766 | rs11153074 | T | G |
| chr6 | 107704813 | rs11153076 | T | G |
| chr6 | 107704933 | rs6903929 | T | A |
| chr6 | 107719440 | rs3844150 | T | A |
| chr6 | 111576375 | rs2235175 | A | C |
| chr6 | 116251808 | rs1931895 | C | G |
| chr6 | 116432987 | rs550373 | G | T |
| chr6 | 116440936 | rs514272 | G | C |
| chr6 | 117560442 | rs1759 | A | T |
| chr6 | 118463267 | rs55868726 | T | A |
| chr6 | 118935008 | rs62422267 | C | G |
| chr6 | 118935067 | rs62422268 | G | C |
| chr6 | 125929251 | rs1138820 | T | A |
| chr6 | 125957084 | rs2295005 | G | C |
| chr6 | 135037429 | rs7742542 | T | G |
| chr6 | 138904788 | rs12619 | G | T |
| chr6 | 143340029 | rs9908 | A | C |
| chr6 | 145886521 | rs2256998 | A | C |
| chr6 | 147388044 | rs7739314 | A | C |
| chr6 | 149594921 | rs9027 | T | A |
| chr6 | 149658547 | rs9322208 | A | T |
| chr6 | 149659317 | rs9393132 | A | C |
| chr6 | 149702212 | rs4870509 | C | G |
| chr6 | 151349037 | rs3734799 | A | C |
| chr6 | 151353191 | rs3823310 | A | C |
| chr6 | 151405040 | rs3757312 | G | T |
| chr6 | 152148053 | rs2252755 | C | G |
| chr6 | 152344126 | rs4645434 | C | A |
| chr6 | 154157305 | rs2236256 | C | A |
| chr6 | 154158261 | rs9322448 | C | G |
| chr6 | 158509664 | rs6918518 | A | C |
| chr6 | 158511785 | rs6880 | G | C |
| chr6 | 158764531 | rs3123101 | T | A |
| chr6 | 159790522 | rs1128661 | T | G |
| chr6 | 166365191 | rs3728 | T | G |
| chr6 | 169708321 | rs3088034 | C | G |
| chr6 | 169709604 | rs7768116 | G | T |
| chr6 | 170578605 | rs3173219 | G | C |
| chr7 | 259884 | rs36170987 | G | T |
| chr7 | 1160151 | rs71518378 | C | A |
| chr7 | 1160154 | rs6946684 | G | C |
| chr7 | 1160155 | rs79849558 | G | C |
| chr7 | 2611534 | rs3823604 | T | G |
| chr7 | 2614158 | rs2272287 | C | A |
| chr7 | 2729301 | rs7805092 | G | T |
| chr7 | 4997828 | rs3087733 | C | G |
| chr7 | 5069139 | rs1127434 | A | C |
| chr7 | 5332775 | rs13238738 | G | T |
| chr7 | 6654579 | rs2464876 | C | A |
| chr7 | 7878420 | rs1558476 | G | C |
| chr7 | 12232942 | rs3800841 | A | T |
| chr7 | 12236419 | rs1468801 | G | C |
| chr7 | 16599990 | rs7156 | A | C |
| chr7 | 16784353 | rs6616 | T | G |
| chr7 | 17814909 | rs2723501 | G | T |
| chr7 | 19696143 | rs3735617 | C | A |
| chr7 | 22944083 | rs4607514 | A | T |
| chr7 | 22945153 | rs10085448 | A | C |
| chr7 | 32495590 | rs56981934 | C | G |
| chr7 | 36153533 | rs66763009 | T | G |
| chr7 | 38257965 | rs7781243 | A | C |
| chr7 | 38377955 | rs2080284 | A | C |
| chr7 | 38723977 | rs17767770 | A | T |
| chr7 | 38725927 | rs3735347 | A | C |
| chr7 | 43877128 | rs2232108 | T | G |
| chr7 | 44040624 | rs149692528 | C | G |
| chr7 | 44044693 | rs4430012 | C | G |
| chr7 | 44768492 | rs1050331 | T | G |
| chr7 | 44769677 | rs1065647 | G | C |
| chr7 | 44885028 | rs6966024 | A | C |
| chr7 | 45183887 | rs3173757 | G | T |
| chr7 | 64349841 | rs663305 | C | A |
| chr7 | 64666158 | rs6460174 | G | C |
| chr7 | 64975111 | rs1060379 | A | T |
| chr7 | 65038404 | rs34438629 | G | T |
| chr7 | 65399999 | rs3846972 | A | C |
| chr7 | 66495270 | rs6460302 | G | C |
| chr7 | 66554403 | rs801209 | G | T |
| chr7 | 66640176 | rs9791712 | C | G |
| chr7 | 66640211 | rs9791713 | C | A |
| chr7 | 74405694 | rs5874 | C | G |
| chr7 | 76066870 | rs3801471 | T | G |
| chr7 | 77094044 | rs3789831 | A | C |
| chr7 | 77780997 | rs6954671 | G | C |
| chr7 | 79460421 | rs7777453 | G | T |
| chr7 | 79461511 | rs4727868 | C | A |
| chr7 | 91873093 | rs9008 | C | G |
| chr7 | 91940976 | rs4727267 | G | C |
| chr7 | 92097613 | rs1063243 | A | C |
| chr7 | 92612319 | rs2285332 | G | C |
| chr7 | 93927805 | rs4261 | A | T |
| chr7 | 94556752 | rs15671 | A | C |
| chr7 | 95584641 | rs11768781 | A | C |
| chr7 | 99456666 | rs1043466 | T | G |
| chr7 | 99891317 | rs1048705 | A | T |
| chr7 | 100119278 | rs3807479 | C | G |
| chr7 | 100214213 | rs1052482 | A | T |
| chr7 | 101138164 | rs7242 | T | G |
| chr7 | 102253445 | rs2529114 | G | T |
| chr7 | 102286955 | rs113764263 | G | C |
| chr7 | 128580057 | rs4294131 | G | T |
| chr7 | 129001172 | rs2305324 | G | C |
| chr7 | 130440971 | rs2287371 | T | G |
| chr7 | 134293326 | rs1862047 | G | C |
| chr7 | 134293415 | rs1862048 | G | C |
| chr7 | 134293592 | rs1862049 | A | T |
| chr7 | 134294473 | rs2241334 | C | G |
| chr7 | 134294793 | rs2504 | A | T |
| chr7 | 135168333 | rs73153794 | A | C |
| chr7 | 135169092 | rs9649052 | C | A |
| chr7 | 137875951 | rs9757 | C | G |
| chr7 | 139045049 | rs10271373 | A | C |
| chr7 | 139047113 | rs10250646 | G | T |
| chr7 | 139778376 | rs1860509 | T | G |
| chr7 | 140085224 | rs10984 | C | A |
| chr7 | 140380287 | rs62490396 | C | G |
| chr7 | 142392270 | rs17208 | C | G |
| chr7 | 143728268 | rs7811904 | T | G |
| chr7 | 143728285 | rs12540107 | G | T |
| chr7 | 143729538 | rs7795149 | C | A |
| chr7 | 148698580 | rs243549 | A | C |
| chr7 | 149182042 | rs1058059 | A | T |
| chr7 | 149254901 | rs1053298 | G | T |
| chr7 | 149282558 | rs3735315 | G | T |
| chr7 | 149282825 | rs4727038 | G | C |
| chr7 | 149861320 | rs2240361 | G | C |
| chr7 | 149866649 | rs3735330 | G | T |
| chr7 | 149880502 | rs1133480 | A | C |
| chr7 | 151012483 | rs7830 | G | T |
| chr7 | 151076479 | rs1050734 | C | A |
| chr7 | 151076720 | rs7262 | A | T |
| chr7 | 151081337 | rs9097 | G | T |
| chr7 | 151213368 | rs2608288 | C | G |
| chr7 | 151234182 | rs2608293 | C | G |
| chr7 | 151556679 | rs1051956 | C | A |
| chr7 | 154944546 | rs2293258 | G | C |
| chr7 | 156969752 | rs3087905 | G | T |
| chr7 | 156971737 | rs6952436 | T | G |
| chr7 | 156972072 | rs3800868 | A | C |
| chr7 | 156972349 | rs7803794 | C | A |
| chr7 | 157857648 | rs12667537 | G | T |
| chr7 | 158732374 | rs3763411 | T | G |
| chr7 | 158733200 | rs34119683 | G | C |
| chr7 | 158741690 | rs59980573 | G | C |
| chr7 | 158945920 | rs2527201 | G | T |
| chr8 | 6414878 | rs2305022 | G | T |
| chr8 | 8893391 | rs3110411 | G | T |
| chr8 | 9137426 | rs12785 | A | T |
| chr8 | 9139426 | rs330915 | T | A |
| chr8 | 9140288 | rs330922 | C | G |
| chr8 | 11304987 | rs2164272 | A | C |
| chr8 | 11324639 | rs6995404 | G | C |
| chr8 | 11326881 | rs13266233 | A | C |
| chr8 | 11327587 | rs1047950 | G | C |
| chr8 | 13133009 | rs13275331 | T | A |
| chr8 | 16140465 | rs4333601 | T | G |
| chr8 | 22251098 | rs9173 | A | C |
| chr8 | 22441144 | rs1049437 | C | A |
| chr8 | 22574864 | rs710098 | C | A |
| chr8 | 23022649 | rs1047275 | G | C |
| chr8 | 25414848 | rs1911251 | C | G |
| chr8 | 27311613 | rs6988218 | A | T |
| chr8 | 27544447 | rs1126452 | A | C |
| chr8 | 27611345 | rs9331888 | C | G |
| chr8 | 28342977 | rs13931 | C | A |
| chr8 | 31116441 | rs1800392 | G | T |
| chr8 | 31141764 | rs1801195 | G | T |
| chr8 | 33567028 | rs3735952 | T | G |
| chr8 | 38996464 | rs7840270 | C | A |
| chr8 | 41578276 | rs999188 | T | G |
| chr8 | 47736128 | rs3614 | A | T |
| chr8 | 60281201 | rs10101374 | T | G |
| chr8 | 68055931 | rs1434774 | C | A |
| chr8 | 81800694 | rs11776932 | A | C |
| chr8 | 86561416 | rs8041 | G | C |
| chr8 | 89934373 | rs1063054 | T | G |
| chr8 | 89935041 | rs2735383 | C | G |
| chr8 | 90623246 | rs4734269 | G | C |
| chr8 | 93729524 | rs2914952 | A | C |
| chr8 | 93733158 | rs16916186 | G | T |
| chr8 | 93924304 | rs911 | G | C |
| chr8 | 94926432 | rs72676983 | A | C |
| chr8 | 96227385 | rs2292836 | A | C |
| chr8 | 103400160 | rs2241777 | C | A |
| chr8 | 103415131 | rs3134295 | A | C |
| chr8 | 107250441 | rs2507800 | T | A |
| chr8 | 107250906 | rs1954727 | C | G |
| chr8 | 109289818 | rs2980619 | T | G |
| chr8 | 109448259 | rs1673407 | G | T |
| chr8 | 109477391 | rs1783148 | A | T |
| chr8 | 115409708 | rs800897 | A | C |
| chr8 | 120537437 | rs3924784 | A | C |
| chr8 | 120537479 | rs3924785 | A | T |
| chr8 | 123436564 | rs6470147 | T | A |
| chr8 | 124450857 | rs3812474 | A | T |
| chr8 | 132812132 | rs235432 | C | A |
| chr8 | 140529755 | rs2944760 | T | G |
| chr8 | 140658761 | rs7460 | A | T |
| chr8 | 141000954 | rs10098028 | C | G |
| chr8 | 141128761 | rs3739232 | C | G |
| chr8 | 141431608 | rs12542151 | G | C |
| chr8 | 141431950 | rs10086164 | T | G |
| chr8 | 142271167 | rs7014279 | A | C |
| chr8 | 142658233 | rs4336593 | T | G |
| chr8 | 142662241 | rs3824208 | G | C |
| chr8 | 142663460 | rs750529 | C | G |
| chr8 | 143636398 | rs11136309 | G | C |
| chr8 | 143693701 | rs6987308 | C | A |
| chr8 | 144379425 | rs6599528 | C | A |
| chr8 | 144850447 | rs1209881 | T | G |
| chr9 | 213810 | rs7850051 | G | C |
| chr9 | 2039983 | rs10964528 | A | C |
| chr9 | 4662369 | rs301487 | A | C |
| chr9 | 4676745 | rs184205 | G | C |
| chr9 | 4711440 | rs6915 | T | A |
| chr9 | 5776236 | rs702274 | C | A |
| chr9 | 15591374 | rs4741510 | T | A |
| chr9 | 19127491 | rs3808660 | G | C |
| chr9 | 21862272 | rs15735 | A | C |
| chr9 | 27326669 | rs1061832 | C | A |
| chr9 | 32526235 | rs3739674 | G | C |
| chr9 | 33025253 | rs2297218 | G | C |
| chr9 | 33473895 | rs2777744 | T | G |
| chr9 | 33921979 | rs2781 | G | C |
| chr9 | 34399004 | rs1002352 | C | A |
| chr9 | 35748809 | rs1570246 | G | T |
| chr9 | 37007478 | rs4880051 | G | T |
| chr9 | 40992306 | rs12376395 | C | A |
| chr9 | 63818436 | rs75137747 | A | C |
| chr9 | 69714063 | rs11139928 | A | T |
| chr9 | 70354601 | rs1052684 | A | T |
| chr9 | 75069774 | rs3752955 | A | C |
| chr9 | 76194562 | rs17179121 | T | G |
| chr9 | 76500928 | rs4532668 | A | C |
| chr9 | 78273375 | rs7859927 | C | A |
| chr9 | 83245613 | rs1408105 | T | A |
| chr9 | 83980816 | rs296890 | C | A |
| chr9 | 83980886 | rs796003 | G | T |
| chr9 | 92297508 | rs710162 | T | A |
| chr9 | 98056788 | rs3199064 | T | G |
| chr9 | 98085269 | rs3780471 | G | T |
| chr9 | 98087218 | rs1059273 | G | T |
| chr9 | 98124543 | rs701379 | A | T |
| chr9 | 105694607 | rs2271247 | A | C |
| chr9 | 109119887 | rs12001627 | G | C |
| chr9 | 112872972 | rs7032763 | A | T |
| chr9 | 112890601 | rs3802491 | G | T |
| chr9 | 113188426 | rs10435864 | A | C |
| chr9 | 113262744 | rs10759637 | A | C |
| chr9 | 113263975 | rs1143245 | G | C |
| chr9 | 114903651 | rs3181368 | A | T |
| chr9 | 120903623 | rs4836834 | T | A |
| chr9 | 120904499 | rs2241003 | G | C |
| chr9 | 121154742 | rs3736855 | T | A |
| chr9 | 122240917 | rs3829097 | T | A |
| chr9 | 125148219 | rs1048251 | G | T |
| chr9 | 125364368 | rs2841333 | G | C |
| chr9 | 126505925 | rs10739677 | T | G |
| chr9 | 127505577 | rs1276 | G | C |
| chr9 | 127867954 | rs4226 | G | T |
| chr9 | 127940874 | rs200385840 | A | C |
| chr9 | 128826609 | rs6478854 | G | C |
| chr9 | 129895273 | rs10760645 | T | A |
| chr9 | 132690283 | rs371222 | C | A |
| chr9 | 132692001 | rs2772006 | T | G |
| chr9 | 132692463 | rs2772005 | C | G |
| chr9 | 133330442 | rs551154 | T | G |
| chr9 | 134026248 | rs417142 | G | T |
| chr9 | 134159126 | rs1128044 | G | C |
| chr9 | 134908885 | rs3012787 | T | G |
| chr9 | 136380752 | rs3812570 | A | C |
| chr9 | 136477334 | rs6560632 | A | C |
| chr10 | 810978 | rs4229 | A | T |
| chr10 | 5094459 | rs12529 | C | G |
| chr10 | 5952731 | rs2296135 | A | C |
| chr10 | 5960405 | rs2228059 | T | G |
| chr10 | 6427193 | rs582052 | G | T |
| chr10 | 12089082 | rs3740015 | T | G |
| chr10 | 12165888 | rs4750179 | A | T |
| chr10 | 12167400 | rs2280619 | C | G |
| chr10 | 14899056 | rs7896464 | T | G |
| chr10 | 16437008 | rs7922050 | C | G |
| chr10 | 17379419 | rs359324 | G | C |
| chr10 | 18651228 | rs3740102 | C | A |
| chr10 | 27014676 | rs2274741 | A | T |
| chr10 | 30311297 | rs540994 | A | C |
| chr10 | 31318302 | rs3737179 | T | G |
| chr10 | 31805962 | rs1023207 | C | A |
| chr10 | 35196021 | rs1057108 | T | G |
| chr10 | 38095087 | rs2472177 | T | G |
| chr10 | 42590065 | rs210284 | G | C |
| chr10 | 42753729 | rs787447 | G | T |
| chr10 | 42831179 | rs7133 | A | C |
| chr10 | 45000672 | rs12269028 | A | T |
| chr10 | 48435527 | rs9284 | T | G |
| chr10 | 49818659 | rs8474 | C | G |
| chr10 | 59906128 | rs1171830 | C | A |
| chr10 | 60794716 | rs10711 | T | G |
| chr10 | 63214777 | rs10761725 | A | T |
| chr10 | 68465747 | rs3758626 | G | T |
| chr10 | 70145813 | rs3750774 | C | A |
| chr10 | 74111977 | rs2131956 | C | G |
| chr10 | 74121589 | rs3180427 | G | T |
| chr10 | 75178505 | rs2804529 | T | A |
| chr10 | 80081936 | rs1932574 | G | T |
| chr10 | 80181161 | rs2573353 | C | A |
| chr10 | 80181251 | rs2788295 | C | G |
| chr10 | 86958679 | rs1800373 | A | C |
| chr10 | 89737874 | rs1062465 | T | A |
| chr10 | 89774767 | rs12948 | G | T |
| chr10 | 91864260 | rs1539042 | C | G |
| chr10 | 95687616 | rs10786229 | A | T |
| chr10 | 96060582 | rs1047370 | G | T |
| chr10 | 96163243 | rs3748226 | T | A |
| chr10 | 97679784 | rs2275047 | G | C |
| chr10 | 97744873 | rs10882993 | G | T |
| chr10 | 100987606 | rs3740484 | G | T |
| chr10 | 101007360 | rs701836 | C | A |
| chr10 | 101007398 | rs14177 | C | G |
| chr10 | 102163139 | rs7897 | G | T |
| chr10 | 103368377 | rs10883859 | T | G |
| chr10 | 103445545 | rs7831 | A | C |
| chr10 | 103596687 | rs10656552 | A | T |
| chr10 | 103918139 | rs4387287 | A | C |
| chr10 | 110510917 | rs1042606 | A | C |
| chr10 | 113729891 | rs10787498 | T | G |
| chr10 | 114436017 | rs1057139 | C | G |
| chr10 | 117374457 | rs3814230 | G | C |
| chr10 | 117375381 | rs183125037 | C | G |
| chr10 | 119677500 | rs8946 | G | C |
| chr10 | 119792069 | rs2289306 | A | C |
| chr10 | 120909270 | rs1045170 | G | T |
| chr10 | 120909289 | rs1045179 | A | C |
| chr10 | 122983379 | rs3736582 | G | C |
| chr10 | 124986120 | rs1046373 | A | C |
| chr10 | 125823221 | rs4385801 | G | T |
| chr10 | 128083514 | rs3210509 | T | A |
| chr10 | 128101514 | rs11106 | G | C |
| chr10 | 131955978 | rs7894 | G | C |
| chr10 | 132330481 | rs1132165 | G | T |
| chr11 | 205198 | rs3782123 | C | A |
| chr11 | 2270485 | rs7126721 | G | T |
| chr11 | 4119902 | rs183484 | C | A |
| chr11 | 4394036 | rs10767979 | A | C |
| chr11 | 5643601 | rs3740998 | C | A |
| chr11 | 5680179 | rs3824949 | G | C |
| chr11 | 6611626 | rs1876300 | A | T |
| chr11 | 6721432 | rs7112649 | G | C |
| chr11 | 7998243 | rs6578918 | C | A |
| chr11 | 9428830 | rs2290423 | T | G |
| chr11 | 9751970 | rs360136 | C | A |
| chr11 | 10878762 | rs11345 | G | T |
| chr11 | 14499808 | rs2575823 | C | A |
| chr11 | 14611024 | rs1403247 | A | C |
| chr11 | 17276818 | rs214087 | G | C |
| chr11 | 18366581 | rs4596 | G | C |
| chr11 | 33075849 | rs7111203 | C | A |
| chr11 | 33076440 | rs2273554 | T | A |
| chr11 | 33707068 | rs831618 | T | G |
| chr11 | 34438925 | rs7943316 | A | T |
| chr11 | 34995658 | rs9326 | G | T |
| chr11 | 43856384 | rs1061810 | C | A |
| chr11 | 44930066 | rs860694 | G | C |
| chr11 | 45882062 | rs2292910 | A | C |
| chr11 | 47426404 | rs7948705 | C | G |
| chr11 | 60389634 | rs2233252 | T | G |
| chr11 | 60415497 | rs7131283 | A | T |
| chr11 | 63614405 | rs3809073 | G | T |
| chr11 | 63827600 | rs8995 | C | A |
| chr11 | 64341646 | rs647152 | T | G |
| chr11 | 64743850 | rs2073798 | T | G |
| chr11 | 65121944 | rs769440 | G | C |
| chr11 | 65775950 | rs522800 | G | C |
| chr11 | 65779386 | rs610037 | A | C |
| chr11 | 66002309 | rs14157 | T | G |
| chr11 | 66002338 | rs1786171 | G | C |
| chr11 | 66537640 | rs1189338 | C | G |
| chr11 | 67437991 | rs869736 | C | A |
| chr11 | 69072944 | rs1466220 | C | G |
| chr11 | 71448718 | rs28364617 | T | G |
| chr11 | 72041114 | rs7115200 | T | G |
| chr11 | 72793803 | rs677231 | A | T |
| chr11 | 73787888 | rs1792174 | T | G |
| chr11 | 74492263 | rs586088 | T | A |
| chr11 | 74641156 | rs1051058 | C | G |
| chr11 | 75566712 | rs650241 | C | G |
| chr11 | 75572608 | rs6704 | C | A |
| chr11 | 77024719 | rs10899344 | T | A |
| chr11 | 78216990 | rs3740677 | G | T |
| chr11 | 82901800 | rs3763814 | C | G |
| chr11 | 82932718 | rs7947780 | G | T |
| chr11 | 88324087 | rs217059 | C | G |
| chr11 | 90197341 | rs7929696 | T | A |
| chr11 | 90202418 | rs1045861 | G | T |
| chr11 | 93147028 | rs7110304 | T | A |
| chr11 | 93729441 | rs7131178 | A | T |
| chr11 | 93763397 | rs666136 | T | A |
| chr11 | 94129327 | rs1138800 | A | C |
| chr11 | 95069457 | rs12627 | C | A |
| chr11 | 95130022 | rs503612 | C | A |
| chr11 | 95130701 | rs677549 | T | G |
| chr11 | 96343125 | rs11021542 | G | C |
| chr11 | 102339006 | rs13711 | A | C |
| chr11 | 107792377 | rs516091 | C | G |
| chr11 | 108121598 | rs3741055 | T | A |
| chr11 | 108121619 | rs3741056 | G | C |
| chr11 | 108368901 | rs4585 | G | T |
| chr11 | 110464278 | rs4753894 | A | C |
| chr11 | 111377789 | rs4622303 | C | G |
| chr11 | 113233274 | rs584427 | T | G |
| chr11 | 113323446 | rs723077 | A | C |
| chr11 | 114399882 | rs3741302 | C | A |
| chr11 | 114410019 | rs13725 | C | G |
| chr11 | 117293108 | rs638405 | C | G |
| chr11 | 118193867 | rs619250 | A | T |
| chr11 | 118229696 | rs869638 | G | C |
| chr11 | 118354737 | rs36061634 | T | A |
| chr11 | 119045044 | rs13929 | G | C |
| chr11 | 119182117 | rs4245191 | C | A |
| chr11 | 119304365 | rs2509671 | C | A |
| chr11 | 120229811 | rs3225 | C | G |
| chr11 | 121577381 | rs2070045 | T | G |
| chr11 | 121605213 | rs3824968 | T | A |
| chr11 | 121632036 | rs1131497 | C | G |
| chr11 | 122812674 | rs3134430 | A | T |
| chr11 | 122872099 | rs67366392 | C | A |
| chr11 | 124146451 | rs1939860 | C | G |
| chr11 | 126263313 | rs9106 | C | A |
| chr11 | 130877336 | rs1050071 | C | G |
| chr11 | 130877491 | rs6590520 | C | G |
| chr11 | 130916450 | rs3751033 | C | A |
| chr11 | 134150327 | rs11223716 | T | G |
| chr12 | 1491812 | rs1064125 | A | T |
| chr12 | 1495324 | rs1046473 | A | C |
| chr12 | 1792319 | rs1044825 | G | T |
| chr12 | 1793600 | rs2058111 | T | G |
| chr12 | 3044528 | rs10431347 | G | T |
| chr12 | 3611779 | rs10848892 | A | T |
| chr12 | 6492009 | rs1048402 | A | C |
| chr12 | 6493530 | rs11545055 | T | A |
| chr12 | 6522003 | rs917634 | C | A |
| chr12 | 6531510 | rs1043271 | T | A |
| chr12 | 6534761 | rs3741915 | T | G |
| chr12 | 6548372 | rs2286724 | T | G |
| chr12 | 6883871 | rs2269357 | A | C |
| chr12 | 6883987 | rs2269358 | G | T |
| chr12 | 7210978 | rs1057225 | C | G |
| chr12 | 8096454 | rs1062836 | C | G |
| chr12 | 9115877 | rs226380 | A | C |
| chr12 | 9657404 | rs17805558 | C | G |
| chr12 | 9660808 | rs34383380 | G | T |
| chr12 | 9693925 | rs7968401 | C | G |
| chr12 | 9699333 | rs1044771 | C | A |
| chr12 | 9753255 | rs917911 | A | C |
| chr12 | 9869549 | rs7313141 | T | G |
| chr12 | 10314934 | rs2537752 | T | A |
| chr12 | 10316507 | rs7301715 | A | T |
| chr12 | 10318718 | rs12813197 | C | G |
| chr12 | 10319739 | rs10845106 | T | G |
| chr12 | 10446203 | rs2734414 | A | T |
| chr12 | 10557664 | rs7971934 | G | C |
| chr12 | 11171577 | rs2416548 | C | A |
| chr12 | 11892330 | rs1062298 | G | T |
| chr12 | 11894839 | rs1051782 | G | C |
| chr12 | 14500733 | rs7955289 | T | A |
| chr12 | 21470188 | rs13035 | T | G |
| chr12 | 25205716 | rs12245 | A | T |
| chr12 | 25205894 | rs12587 | T | G |
| chr12 | 25206035 | rs1137196 | T | G |
| chr12 | 25206394 | rs1137189 | A | T |
| chr12 | 26336611 | rs1049380 | G | T |
| chr12 | 27799687 | rs17801400 | T | G |
| chr12 | 27802908 | rs9029 | C | G |
| chr12 | 29338198 | rs11050203 | A | T |
| chr12 | 30630250 | rs4082413 | C | G |
| chr12 | 31385426 | rs7294574 | G | T |
| chr12 | 32642025 | rs7980205 | T | G |
| chr12 | 32644303 | rs11052123 | G | T |
| chr12 | 32792173 | rs12612 | G | C |
| chr12 | 40320032 | rs1427263 | C | A |
| chr12 | 40368129 | rs10878441 | A | C |
| chr12 | 42158495 | rs2406568 | G | C |
| chr12 | 46184372 | rs3742059 | A | C |
| chr12 | 46268702 | rs2242355 | G | C |
| chr12 | 47968629 | rs6823 | G | C |
| chr12 | 48341521 | rs2634679 | G | T |
| chr12 | 48689611 | rs3209584 | G | T |
| chr12 | 48921079 | rs10875894 | C | A |
| chr12 | 49188909 | rs1039225 | T | G |
| chr12 | 50744904 | rs2280503 | A | C |
| chr12 | 51059583 | rs3190077 | A | C |
| chr12 | 51061621 | rs7722 | C | A |
| chr12 | 51061956 | rs2306732 | G | T |
| chr12 | 56433910 | rs2279665 | C | G |
| chr12 | 56594558 | rs9368 | C | A |
| chr12 | 56739356 | rs1131514 | T | G |
| chr12 | 57723954 | rs238517 | T | G |
| chr12 | 59782798 | rs10877338 | A | C |
| chr12 | 62335441 | rs2242032 | G | C |
| chr12 | 63144342 | rs10047514 | A | C |
| chr12 | 64410018 | rs11175383 | A | C |
| chr12 | 64482007 | rs7486100 | T | A |
| chr12 | 64697534 | rs15958 | T | G |
| chr12 | 65463775 | rs7316024 | T | A |
| chr12 | 68432609 | rs3741807 | G | T |
| chr12 | 69273295 | rs1463335 | T | A |
| chr12 | 71786392 | rs328742 | G | T |
| chr12 | 79592094 | rs2307220 | A | C |
| chr12 | 88496200 | rs1907699 | A | T |
| chr12 | 95972991 | rs1059844 | T | G |
| chr12 | 98515034 | rs11768 | T | G |
| chr12 | 101726511 | rs7965541 | C | A |
| chr12 | 103957073 | rs703657 | T | A |
| chr12 | 104287004 | rs11111979 | C | G |
| chr12 | 105236087 | rs1196785 | C | G |
| chr12 | 109052491 | rs12426673 | G | T |
| chr12 | 109536174 | rs1045255 | G | C |
| chr12 | 111599196 | rs695871 | G | C |
| chr12 | 113010847 | rs13311 | C | A |
| chr12 | 113057821 | rs3741985 | G | C |
| chr12 | 117030562 | rs2242469 | C | G |
| chr12 | 120904130 | rs2393716 | C | G |
| chr12 | 121777720 | rs15797 | C | A |
| chr12 | 122143969 | rs1047813 | A | T |
| chr12 | 122327956 | rs1129167 | G | C |
| chr12 | 122361151 | rs79909185 | C | A |
| chr12 | 122716390 | rs1696352 | T | G |
| chr12 | 122985100 | rs3741530 | G | T |
| chr12 | 123156117 | rs1727314 | C | A |
| chr12 | 123257546 | rs1533703 | T | G |
| chr12 | 123411359 | rs28577594 | G | C |
| chr12 | 130789849 | rs1236 | A | T |
| chr12 | 132189489 | rs7307636 | G | C |
| chr12 | 133106694 | rs905225 | A | T |
| chr12 | 133107042 | rs1025 | A | T |
| chr12 | 133107164 | rs1026 | C | A |
| chr13 | 20782511 | rs4617691 | T | A |
| chr13 | 24303412 | rs9580931 | G | C |
| chr13 | 24435159 | rs1050112 | G | T |
| chr13 | 24435347 | rs1050110 | C | G |
| chr13 | 25249069 | rs7999040 | T | A |
| chr13 | 28700517 | rs1771162 | G | C |
| chr13 | 30206974 | rs9506275 | C | A |
| chr13 | 32402511 | rs61946986 | G | C |
| chr13 | 39655820 | rs3812883 | T | A |
| chr13 | 40808575 | rs17849654 | A | T |
| chr13 | 42992237 | rs3825511 | A | C |
| chr13 | 44989329 | rs1140993 | G | C |
| chr13 | 45333603 | rs7316959 | A | C |
| chr13 | 48709632 | rs1323552 | A | C |
| chr13 | 49444706 | rs61959991 | T | G |
| chr13 | 49533239 | rs1062979 | G | C |
| chr13 | 49533837 | rs3186012 | G | C |
| chr13 | 52028783 | rs3825528 | A | C |
| chr13 | 52029058 | rs3742289 | G | T |
| chr13 | 52697614 | rs7324427 | G | C |
| chr13 | 67228207 | rs8000556 | A | T |
| chr13 | 72775221 | rs7332388 | G | C |
| chr13 | 78614399 | rs1044385 | T | A |
| chr13 | 79313276 | rs1748768 | A | T |
| chr13 | 98793610 | rs2899 | A | T |
| chr13 | 102875652 | rs17655 | G | C |
| chr13 | 110713558 | rs2289461 | G | C |
| chr13 | 113457972 | rs3814254 | C | A |
| chr14 | 20316559 | rs1132644 | G | T |
| chr14 | 20404722 | rs1760898 | G | T |
| chr14 | 20920107 | rs3748340 | G | C |
| chr14 | 21090399 | rs6571653 | G | C |
| chr14 | 22894328 | rs4982704 | C | A |
| chr14 | 23098565 | rs6736 | T | A |
| chr14 | 23475305 | rs2236261 | C | A |
| chr14 | 23968980 | rs4706 | C | A |
| chr14 | 24432043 | rs3742520 | A | C |
| chr14 | 31446699 | rs7153450 | A | T |
| chr14 | 34711183 | rs712301 | T | A |
| chr14 | 35046893 | rs799474 | C | G |
| chr14 | 39308472 | rs1950952 | G | C |
| chr14 | 39399442 | rs3814860 | C | A |
| chr14 | 49633965 | rs2985686 | C | G |
| chr14 | 50758414 | rs2073349 | G | T |
| chr14 | 55047130 | rs11849878 | G | C |
| chr14 | 55367156 | rs1572611 | T | A |
| chr14 | 56299376 | rs8018553 | T | G |
| chr14 | 59458941 | rs9323348 | G | T |
| chr14 | 64055956 | rs8010911 | G | C |
| chr14 | 64170429 | rs7161192 | C | A |
| chr14 | 64225659 | rs1152583 | C | A |
| chr14 | 64533320 | rs1542313 | A | C |
| chr14 | 64793509 | rs229591 | T | G |
| chr14 | 64946030 | rs3087955 | G | C |
| chr14 | 65084098 | rs7159443 | T | A |
| chr14 | 65742472 | rs1054218 | C | G |
| chr14 | 66013530 | rs1807441 | A | C |
| chr14 | 67471175 | rs1315732 | A | C |
| chr14 | 67650289 | rs10483801 | C | A |
| chr14 | 70372672 | rs11844845 | A | C |
| chr14 | 71112418 | rs221926 | A | C |
| chr14 | 73718186 | rs4903144 | G | C |
| chr14 | 74064782 | rs3815330 | T | G |
| chr14 | 74661613 | rs16661 | A | C |
| chr14 | 74663532 | rs1045430 | T | G |
| chr14 | 74713031 | rs2270425 | C | G |
| chr14 | 75009368 | rs4556 | G | C |
| chr14 | 75124143 | rs175449 | A | T |
| chr14 | 75428242 | rs113661747 | C | G |
| chr14 | 76202966 | rs4903385 | C | A |
| chr14 | 77335311 | rs6636 | G | C |
| chr14 | 77507838 | rs11159268 | C | A |
| chr14 | 88012710 | rs12878534 | A | T |
| chr14 | 89160210 | rs11159889 | T | G |
| chr14 | 92164621 | rs7142318 | T | A |
| chr14 | 95408171 | rs1047403 | C | G |
| chr14 | 95411670 | rs10047824 | A | C |
| chr14 | 95412071 | rs4905299 | A | C |
| chr14 | 95457333 | rs2024863 | A | C |
| chr14 | 95756165 | rs4359368 | C | A |
| chr14 | 96364089 | rs57280159 | G | C |
| chr14 | 100306335 | rs11557209 | G | C |
| chr14 | 102499100 | rs3783382 | A | T |
| chr14 | 103521843 | rs1136165 | G | T |
| chr14 | 103629432 | rs3742463 | G | T |
| chr14 | 104927219 | rs2841280 | G | C |
| chr14 | 105588091 | rs9972103 | C | G |
| chr15 | 22671530 | rs389677 | G | T |
| chr15 | 22825366 | rs1059774 | C | G |
| chr15 | 22912200 | rs2289818 | C | G |
| chr15 | 28755672 | rs422339 | C | A |
| chr15 | 29117870 | rs3751555 | G | C |
| chr15 | 34853939 | rs1357180 | T | A |
| chr15 | 40091578 | rs3743129 | A | C |
| chr15 | 40419071 | rs2075625 | C | G |
| chr15 | 40459356 | rs3803357 | C | A |
| chr15 | 41342390 | rs7178777 | C | A |
| chr15 | 41898869 | rs7166358 | C | A |
| chr15 | 42415645 | rs1062038 | G | C |
| chr15 | 42567037 | rs10851411 | T | G |
| chr15 | 42736551 | rs4265781 | T | A |
| chr15 | 43408732 | rs1058298 | G | T |
| chr15 | 48989968 | rs11542124 | T | G |
| chr15 | 49033092 | rs11638215 | A | C |
| chr15 | 49934116 | rs2452524 | G | T |
| chr15 | 51737823 | rs28699115 | G | T |
| chr15 | 51810635 | rs2554315 | T | G |
| chr15 | 56918429 | rs2165461 | G | C |
| chr15 | 59055730 | rs1446239 | C | A |
| chr15 | 59659798 | rs1046053 | C | A |
| chr15 | 59659925 | rs6494133 | G | T |
| chr15 | 59660054 | rs4775195 | C | G |
| chr15 | 59662137 | rs6151589 | C | A |
| chr15 | 60492410 | rs7165874 | A | T |
| chr15 | 61853956 | rs2059471 | A | C |
| chr15 | 63542120 | rs1421151 | A | T |
| chr15 | 63594180 | rs11457 | G | C |
| chr15 | 64154773 | rs895885 | C | G |
| chr15 | 65624189 | rs3743171 | A | T |
| chr15 | 65792069 | rs1369312 | G | T |
| chr15 | 67201966 | rs8991 | T | G |
| chr15 | 74843920 | rs6938 | C | G |
| chr15 | 76434124 | rs1607017 | G | T |
| chr15 | 77052451 | rs11737 | T | A |
| chr15 | 77484156 | rs952471 | C | G |
| chr15 | 77484220 | rs952472 | A | C |
| chr15 | 77996436 | rs56367308 | G | T |
| chr15 | 78944838 | rs1036937 | C | A |
| chr15 | 79897181 | rs2903105 | C | G |
| chr15 | 81001003 | rs111785807 | C | G |
| chr15 | 85581252 | rs4843074 | C | G |
| chr15 | 85583044 | rs4842891 | C | A |
| chr15 | 88907356 | rs1878326 | G | T |
| chr15 | 90885359 | rs7183988 | T | G |
| chr15 | 92171901 | rs2270061 | A | T |
| chr15 | 93025654 | rs9672839 | A | C |
| chr15 | 94340879 | rs8025851 | G | C |
| chr15 | 97973392 | rs1043374 | A | C |
| chr15 | 99712600 | rs325400 | G | T |
| chr15 | 100569472 | rs8451 | C | A |
| chr15 | 100569589 | rs12157 | C | G |
| chr15 | 100570060 | rs2411836 | T | A |
| chr15 | 100573111 | rs7174482 | C | G |
| chr15 | 101071602 | rs12911171 | A | C |
| chr15 | 101072338 | rs7179909 | A | T |
| chr15 | 101489392 | rs1135910 | C | G |
| chr16 | 84442 | rs1061435 | C | A |
| chr16 | 553884 | rs11539618 | C | G |
| chr16 | 554283 | rs11539619 | G | T |
| chr16 | 627854 | rs15564 | G | T |
| chr16 | 668514 | rs7204542 | C | G |
| chr16 | 1493567 | rs2272972 | C | G |
| chr16 | 1674692 | rs2294444 | G | T |
| chr16 | 1786795 | rs2235648 | C | A |
| chr16 | 1997890 | rs9081 | C | A |
| chr16 | 2267777 | rs11642797 | T | G |
| chr16 | 2762938 | rs2240140 | C | A |
| chr16 | 2832196 | rs12373 | G | T |
| chr16 | 2912037 | rs71384679 | C | G |
| chr16 | 3382594 | rs1044390 | T | A |
| chr16 | 4434395 | rs1139653 | A | T |
| chr16 | 4510928 | rs7702 | G | C |
| chr16 | 4848119 | rs2219271 | C | G |
| chr16 | 8774919 | rs1641022 | C | A |
| chr16 | 8781688 | rs737695 | G | C |
| chr16 | 8782001 | rs1641031 | A | C |
| chr16 | 8782345 | rs3743801 | C | G |
| chr16 | 8782420 | rs4985000 | G | C |
| chr16 | 8783997 | rs12597124 | C | G |
| chr16 | 9109737 | rs9940147 | T | A |
| chr16 | 9109791 | rs9937728 | A | C |
| chr16 | 11742542 | rs3743587 | C | G |
| chr16 | 11836480 | rs3743590 | C | A |
| chr16 | 11871533 | rs11641520 | C | G |
| chr16 | 12568729 | rs1075844 | A | C |
| chr16 | 12569607 | rs745828 | T | A |
| chr16 | 12571072 | rs3826103 | A | C |
| chr16 | 13948831 | rs3743538 | G | T |
| chr16 | 17104667 | rs9934313 | C | A |
| chr16 | 20733933 | rs1058905 | A | C |
| chr16 | 22285165 | rs2290829 | C | A |
| chr16 | 28496323 | rs180743 | C | G |
| chr16 | 30506720 | rs2230433 | G | C |
| chr16 | 48540129 | rs3743779 | T | G |
| chr16 | 48540726 | rs1039340 | A | C |
| chr16 | 50732216 | rs3135499 | A | C |
| chr16 | 53388447 | rs2908796 | T | G |
| chr16 | 56346681 | rs2550299 | C | G |
| chr16 | 57663656 | rs10852555 | C | A |
| chr16 | 69700600 | rs1865965 | C | A |
| chr16 | 70158561 | rs55679539 | A | C |
| chr16 | 70162283 | rs1044876 | T | G |
| chr16 | 70529184 | rs76371422 | C | G |
| chr16 | 71856586 | rs2291947 | C | G |
| chr16 | 71949873 | rs1035543 | G | C |
| chr16 | 72008783 | rs3213422 | A | C |
| chr16 | 72096304 | rs1050361 | C | G |
| chr16 | 72105285 | rs2074626 | C | A |
| chr16 | 72112542 | rs7940 | C | G |
| chr16 | 74623587 | rs8058133 | A | T |
| chr16 | 75445408 | rs59347518 | C | G |
| chr16 | 75464355 | rs34904236 | G | T |
| chr16 | 75612787 | rs3743598 | G | T |
| chr16 | 77193934 | rs3743760 | G | T |
| chr16 | 77212950 | rs2278048 | T | G |
| chr16 | 78996264 | rs80205998 | C | A |
| chr16 | 79211923 | rs383362 | G | T |
| chr16 | 80602400 | rs33943240 | C | G |
| chr16 | 80602910 | rs3045223 | C | A |
| chr16 | 81631447 | rs4265801 | T | G |
| chr16 | 81739421 | rs12446781 | G | C |
| chr16 | 83805782 | rs42763 | G | C |
| chr16 | 84479791 | rs1044871 | A | T |
| chr16 | 84489291 | rs436278 | G | C |
| chr16 | 84616326 | rs2967868 | A | C |
| chr16 | 84664100 | rs873857 | G | C |
| chr16 | 84664602 | rs881584 | C | G |
| chr16 | 84872492 | rs721005 | C | G |
| chr16 | 85921698 | rs1568391 | G | T |
| chr16 | 85935402 | rs385989 | T | G |
| chr16 | 86531065 | rs1046200 | G | T |
| chr16 | 87830869 | rs1060266 | G | C |
| chr16 | 87832532 | rs1060253 | G | C |
| chr16 | 88717041 | rs8057031 | C | G |
| chr16 | 89323224 | rs3114901 | A | C |
| chr16 | 89696951 | rs3803690 | G | C |
| chr16 | 89798695 | rs11076626 | T | A |
| chr17 | 2299873 | rs216195 | T | G |
| chr17 | 3861974 | rs2915546 | T | G |
| chr17 | 4006110 | rs1052617 | C | A |
| chr17 | 4157188 | rs1049523 | G | T |
| chr17 | 4269648 | rs1045738 | C | A |
| chr17 | 5093744 | rs3744706 | G | C |
| chr17 | 5384859 | rs10792 | A | T |
| chr17 | 5385474 | rs1058400 | G | C |
| chr17 | 5422825 | rs12761 | C | G |
| chr17 | 6454782 | rs4796500 | C | G |
| chr17 | 6620978 | rs9889363 | T | A |
| chr17 | 6657372 | rs2309597 | T | G |
| chr17 | 6760576 | rs2271231 | C | G |
| chr17 | 7587859 | rs4227 | G | T |
| chr17 | 8189376 | rs8531 | T | G |
| chr17 | 9913073 | rs15814 | G | T |
| chr17 | 9913314 | rs3177567 | G | C |
| chr17 | 9914873 | rs9900085 | A | C |
| chr17 | 9915653 | rs1047365 | T | A |
| chr17 | 10680397 | rs7512 | G | C |
| chr17 | 12992667 | rs1044564 | G | C |
| chr17 | 13865219 | rs11651470 | C | A |
| chr17 | 14347038 | rs2200000 | T | G |
| chr17 | 15230858 | rs13422 | T | G |
| chr17 | 15717765 | rs62071728 | A | C |
| chr17 | 17142710 | rs3744137 | C | A |
| chr17 | 17793217 | rs3803763 | G | C |
| chr17 | 17793441 | rs11649804 | C | A |
| chr17 | 18314850 | rs2273030 | A | C |
| chr17 | 18325291 | rs4925172 | C | A |
| chr17 | 18326138 | rs12949119 | T | A |
| chr17 | 18672943 | rs4924901 | G | C |
| chr17 | 20056515 | rs4005937 | A | C |
| chr17 | 27456509 | rs114378193 | C | G |
| chr17 | 27893329 | rs4063521 | G | T |
| chr17 | 28396594 | rs2239911 | G | T |
| chr17 | 30526512 | rs216463 | A | C |
| chr17 | 31376420 | rs1800845 | C | G |
| chr17 | 31536936 | rs1551358 | G | T |
| chr17 | 34962918 | rs8249 | A | T |
| chr17 | 35268823 | rs2622524 | T | G |
| chr17 | 35363447 | rs12453150 | C | A |
| chr17 | 35422900 | rs1849733 | A | C |
| chr17 | 35470352 | rs9916257 | G | T |
| chr17 | 35548243 | rs8073060 | T | A |
| chr17 | 36544987 | rs3736166 | C | G |
| chr17 | 37517559 | rs11868673 | T | A |
| chr17 | 38770478 | rs228289 | T | G |
| chr17 | 39727784 | rs1058808 | C | G |
| chr17 | 42554255 | rs676387 | C | A |
| chr17 | 42562786 | rs615942 | C | A |
| chr17 | 43022008 | rs2070835 | A | C |
| chr17 | 43148782 | rs11079056 | C | A |
| chr17 | 43218965 | rs35989681 | C | A |
| chr17 | 43361038 | rs60766100 | G | T |
| chr17 | 44177159 | rs7217858 | T | G |
| chr17 | 45023913 | rs7225735 | A | C |
| chr17 | 45051538 | rs8071429 | T | A |
| chr17 | 46548562 | rs1863115 | C | A |
| chr17 | 46941877 | rs1047779 | T | G |
| chr17 | 47925378 | rs1130932 | G | T |
| chr17 | 47947294 | rs7220104 | A | C |
| chr17 | 48107652 | rs2072441 | C | G |
| chr17 | 49290174 | rs3179840 | T | G |
| chr17 | 50360694 | rs2526537 | G | T |
| chr17 | 50693774 | rs9455 | G | T |
| chr17 | 51178613 | rs3744661 | C | G |
| chr17 | 58091352 | rs12950704 | G | C |
| chr17 | 59399874 | rs1451508 | T | G |
| chr17 | 63689097 | rs16947042 | T | G |
| chr17 | 67071095 | rs16960542 | A | T |
| chr17 | 67073202 | rs7212626 | A | C |
| chr17 | 68127291 | rs8064704 | T | G |
| chr17 | 68206978 | rs9892851 | T | G |
| chr17 | 68271550 | rs7222013 | A | T |
| chr17 | 69516862 | rs1133228 | C | A |
| chr17 | 73248027 | rs1472454 | C | G |
| chr17 | 74522104 | rs72852234 | A | C |
| chr17 | 74776559 | rs4789096 | G | C |
| chr17 | 75063725 | rs4365317 | C | G |
| chr17 | 75499611 | rs13357 | C | G |
| chr17 | 75776775 | rs7342 | G | C |
| chr17 | 75953459 | rs1135640 | G | C |
| chr17 | 77089178 | rs2247814 | C | G |
| chr17 | 80319389 | rs55996424 | A | T |
| chr17 | 80332302 | rs9913636 | G | C |
| chr17 | 80332508 | rs9908287 | C | G |
| chr17 | 81029363 | rs113473934 | C | G |
| chr17 | 81222862 | rs9911096 | C | G |
| chr17 | 81228529 | rs1048775 | G | C |
| chr17 | 81246424 | rs2725405 | G | C |
| chr17 | 81558092 | rs6565596 | T | G |
| chr17 | 82022880 | rs3934983 | C | A |
| chr17 | 82458214 | rs28365943 | C | G |
| chr18 | 2547501 | rs2677879 | G | T |
| chr18 | 3013288 | rs28738097 | C | G |
| chr18 | 3246488 | rs1055549 | T | G |
| chr18 | 3247258 | rs4798075 | A | C |
| chr18 | 5238443 | rs11795 | G | C |
| chr18 | 5239337 | rs3170041 | T | G |
| chr18 | 5289888 | rs2789 | C | G |
| chr18 | 5392654 | rs9953490 | T | A |
| chr18 | 9957576 | rs29068 | C | A |
| chr18 | 12329537 | rs1129115 | C | G |
| chr18 | 13651498 | rs9945994 | C | A |
| chr18 | 32131298 | rs1054667 | A | C |
| chr18 | 35142849 | rs617849 | G | C |
| chr18 | 35246672 | rs1060758 | G | T |
| chr18 | 35246697 | rs1060760 | T | A |
| chr18 | 36138363 | rs1785934 | A | C |
| chr18 | 42084140 | rs484350 | A | T |
| chr18 | 45750931 | rs9954521 | T | A |
| chr18 | 45752515 | rs3178156 | A | C |
| chr18 | 45984012 | rs6507658 | G | C |
| chr18 | 45984961 | rs1438388 | G | C |
| chr18 | 45985229 | rs1048827 | G | T |
| chr18 | 47836843 | rs1792666 | A | T |
| chr18 | 57029213 | rs3826642 | C | A |
| chr18 | 57601254 | rs11356 | A | C |
| chr18 | 63317731 | rs1893806 | C | A |
| chr18 | 63367187 | rs402348 | T | G |
| chr18 | 69860524 | rs1790947 | T | G |
| chr18 | 80045856 | rs3744872 | A | C |
| chr19 | 973971 | rs12971369 | T | A |
| chr19 | 984554 | rs4806884 | C | G |
| chr19 | 1065564 | rs2242437 | G | C |
| chr19 | 1854152 | rs12972720 | G | C |
| chr19 | 1877728 | rs2289287 | G | T |
| chr19 | 1924654 | rs3810415 | C | A |
| chr19 | 3121910 | rs308040 | C | G |
| chr19 | 3209485 | rs4594 | T | G |
| chr19 | 3592857 | rs10411250 | A | C |
| chr19 | 4653358 | rs4806994 | C | G |
| chr19 | 6494904 | rs3099129 | C | G |
| chr19 | 8526688 | rs2303687 | C | G |
| chr19 | 10112159 | rs1037686 | T | A |
| chr19 | 10468798 | rs7256672 | T | G |
| chr19 | 10489766 | rs1048290 | G | C |
| chr19 | 10559508 | rs3826709 | C | G |
| chr19 | 10653527 | rs4804514 | G | T |
| chr19 | 11354640 | rs6887 | G | C |
| chr19 | 12431840 | rs28599549 | T | A |
| chr19 | 13152241 | rs55724477 | C | G |
| chr19 | 14031804 | rs6511905 | C | G |
| chr19 | 14719756 | rs11666622 | G | T |
| chr19 | 15122770 | rs2074265 | C | A |
| chr19 | 15660440 | rs28371514 | T | G |
| chr19 | 15660443 | rs28371515 | G | C |
| chr19 | 15661423 | rs1063803 | T | G |
| chr19 | 15661567 | rs1140862 | T | A |
| chr19 | 15661689 | rs4305201 | T | A |
| chr19 | 15661754 | rs4358060 | T | A |
| chr19 | 17283695 | rs891017 | A | C |
| chr19 | 17286692 | rs1465582 | T | G |
| chr19 | 17286891 | rs10401700 | A | C |
| chr19 | 17377332 | rs10417806 | A | C |
| chr19 | 18427932 | rs10405636 | A | C |
| chr19 | 19338877 | rs2074090 | G | T |
| chr19 | 21058731 | rs10409844 | T | A |
| chr19 | 21423627 | rs4621113 | G | T |
| chr19 | 23261681 | rs3180232 | A | T |
| chr19 | 23359489 | rs385750 | G | C |
| chr19 | 34950545 | rs7250359 | T | G |
| chr19 | 34963700 | rs2546028 | A | C |
| chr19 | 35232731 | rs10416254 | G | T |
| chr19 | 36324999 | rs2972629 | G | T |
| chr19 | 36325162 | rs1127406 | T | G |
| chr19 | 36512705 | rs2945977 | A | T |
| chr19 | 36545166 | rs3096637 | T | G |
| chr19 | 36951549 | rs826303 | C | A |
| chr19 | 38878729 | rs2015 | T | G |
| chr19 | 38915527 | rs9403 | C | G |
| chr19 | 41426179 | rs284660 | G | T |
| chr19 | 41811262 | rs2008808 | T | G |
| chr19 | 43475437 | rs1055099 | G | T |
| chr19 | 44007050 | rs2356549 | A | T |
| chr19 | 44477666 | rs1897820 | G | C |
| chr19 | 44747899 | rs2965169 | A | C |
| chr19 | 45365051 | rs238406 | T | G |
| chr19 | 45940628 | rs1047061 | C | A |
| chr19 | 46023040 | rs2072562 | T | G |
| chr19 | 46839610 | rs312185 | A | C |
| chr19 | 47082260 | rs7250850 | G | C |
| chr19 | 47275600 | rs6612 | C | G |
| chr19 | 47352883 | rs1064202 | G | C |
| chr19 | 48151296 | rs20580 | G | T |
| chr19 | 48208649 | rs4597433 | T | A |
| chr19 | 48208827 | rs118114021 | A | T |
| chr19 | 48256721 | rs12459322 | C | G |
| chr19 | 48257419 | rs7343088 | A | T |
| chr19 | 48321846 | rs10403090 | G | C |
| chr19 | 48469282 | rs1799257 | A | C |
| chr19 | 49451759 | rs2293011 | G | T |
| chr19 | 49659652 | rs7251 | C | G |
| chr19 | 49665670 | rs2304205 | A | C |
| chr19 | 49877601 | rs731826 | T | G |
| chr19 | 50725545 | rs1053020 | T | G |
| chr19 | 50820217 | rs5516 | C | G |
| chr19 | 51127225 | rs2258983 | C | A |
| chr19 | 51795323 | rs12610825 | A | C |
| chr19 | 51992393 | rs11084128 | A | T |
| chr19 | 51992431 | rs2288886 | A | T |
| chr19 | 52384174 | rs8104808 | A | C |
| chr19 | 52385367 | rs3170100 | T | G |
| chr19 | 52592134 | rs7245397 | T | A |
| chr19 | 52592163 | rs7259768 | A | T |
| chr19 | 52800452 | rs10417163 | T | G |
| chr19 | 52905530 | rs28538829 | G | C |
| chr19 | 52908094 | rs7256037 | C | A |
| chr19 | 52949691 | rs1808106 | T | G |
| chr19 | 52951536 | rs12459008 | A | T |
| chr19 | 53202556 | rs11084224 | G | C |
| chr19 | 53211443 | rs11672910 | C | A |
| chr19 | 53211614 | rs4801970 | C | G |
| chr19 | 53383782 | rs1817396 | C | A |
| chr19 | 53385373 | rs2708712 | T | G |
| chr19 | 53441861 | rs4803124 | G | C |
| chr19 | 53441997 | rs4803126 | A | T |
| chr19 | 53454872 | rs2708743 | T | G |
| chr19 | 53456314 | rs2617726 | G | C |
| chr19 | 54106385 | rs254266 | T | G |
| chr19 | 54354020 | rs111919294 | T | A |
| chr19 | 54632040 | rs1061681 | T | G |
| chr19 | 55000864 | rs1043673 | C | A |
| chr19 | 55015005 | rs2304166 | G | C |
| chr19 | 55321059 | rs10412726 | T | G |
| chr19 | 55461583 | rs2303088 | T | G |
| chr19 | 56664936 | rs12460400 | T | G |
| chr19 | 57320721 | rs4801461 | G | T |
| chr19 | 57326650 | rs6510057 | C | G |
| chr19 | 57328199 | rs1968090 | T | A |
| chr19 | 57363586 | rs2285604 | C | G |
| chr19 | 57471570 | rs2885061 | C | G |
| chr19 | 57472543 | rs10405925 | C | A |
| chr19 | 57473058 | rs10407042 | C | A |
| chr19 | 57494300 | rs7248267 | C | A |
| chr19 | 57593078 | rs58449774 | G | C |
| chr19 | 57689260 | rs12608585 | G | T |
| chr19 | 57757805 | rs13037 | G | C |
| chr19 | 57849960 | rs28374851 | G | C |
| chr19 | 57862267 | rs3745134 | C | G |
| chr19 | 58315169 | rs3206947 | T | A |
| chr19 | 58417938 | rs3764531 | G | C |
| chr19 | 58478128 | rs893185 | A | C |
| chr19 | 58582117 | rs3499 | G | T |
| chr19 | 58583086 | rs3211055 | A | C |
| chr20 | 437555 | rs3746793 | T | A |
| chr20 | 1442888 | rs3210915 | A | T |
| chr20 | 1443203 | rs13063 | G | T |
| chr20 | 1467296 | rs3795134 | C | G |
| chr20 | 1477265 | rs6135048 | C | A |
| chr20 | 1937841 | rs3197744 | G | T |
| chr20 | 3650034 | rs12930 | A | C |
| chr20 | 3867769 | rs16989000 | A | C |
| chr20 | 3929522 | rs7270329 | G | C |
| chr20 | 3931990 | rs397095 | G | T |
| chr20 | 3931991 | rs443168 | C | G |
| chr20 | 3932476 | rs241604 | G | T |
| chr20 | 4856675 | rs6037992 | G | C |
| chr20 | 5192362 | rs6133193 | G | C |
| chr20 | 5544961 | rs6107649 | A | C |
| chr20 | 7980265 | rs6055433 | A | C |
| chr20 | 16050724 | rs16997014 | G | C |
| chr20 | 17494045 | rs6105762 | T | G |
| chr20 | 18484357 | rs5867 | C | A |
| chr20 | 23376692 | rs2424527 | A | C |
| chr20 | 25058203 | rs3646 | C | G |
| chr20 | 25300548 | rs11100 | G | C |
| chr20 | 32194740 | rs1056776 | C | G |
| chr20 | 32333144 | rs2151437 | A | C |
| chr20 | 33667025 | rs7263119 | G | T |
| chr20 | 37316835 | rs1043415 | C | G |
| chr20 | 38926473 | rs3752290 | G | C |
| chr20 | 46014194 | rs13969 | A | C |
| chr20 | 46062711 | rs1537028 | T | G |
| chr20 | 49255840 | rs238221 | C | G |
| chr20 | 49635801 | rs235034 | T | A |
| chr20 | 50889658 | rs875068 | C | G |
| chr20 | 51004246 | rs1054268 | G | T |
| chr20 | 51599747 | rs3827044 | A | C |
| chr20 | 56458420 | rs3746623 | C | G |
| chr20 | 57604366 | rs6064572 | C | A |
| chr20 | 57607240 | rs6123711 | A | C |
| chr20 | 58361815 | rs6026214 | C | A |
| chr20 | 58362977 | rs968323 | T | G |
| chr20 | 58365097 | rs6026220 | A | C |
| chr20 | 62650103 | rs3901528 | G | T |
| chr20 | 62650521 | rs3843758 | A | T |
| chr20 | 62800205 | rs7397 | A | C |
| chr20 | 63104157 | rs750698 | T | G |
| chr20 | 63562677 | rs3810483 | G | C |
| chr20 | 63641230 | rs3865523 | G | T |
| chr20 | 63966341 | rs817329 | T | G |
| chr21 | 17792211 | rs1062204 | C | G |
| chr21 | 26466883 | rs219639 | C | G |
| chr21 | 28577229 | rs2831900 | T | A |
| chr21 | 33449384 | rs1044213 | G | C |
| chr21 | 34792108 | rs13051066 | G | T |
| chr21 | 37065463 | rs7337 | C | G |
| chr21 | 39192959 | rs2836934 | A | C |
| chr21 | 41426246 | rs464138 | A | C |
| chr21 | 41987926 | rs693386 | C | A |
| chr21 | 42769062 | rs3087994 | A | C |
| chr21 | 42873634 | rs2248490 | C | G |
| chr21 | 43032758 | rs2839628 | C | G |
| chr21 | 43693748 | rs762400 | C | G |
| chr21 | 44339314 | rs73374031 | G | C |
| chr21 | 45514947 | rs1051296 | A | C |
| chr21 | 46285759 | rs17182538 | C | A |
| chr22 | 17114180 | rs5992628 | T | G |
| chr22 | 17149596 | rs1034859 | C | A |
| chr22 | 17181273 | rs7290147 | C | G |
| chr22 | 18089340 | rs456551 | T | A |
| chr22 | 18096995 | rs468784 | C | A |
| chr22 | 19919576 | rs5748469 | C | A |
| chr22 | 20064958 | rs3804043 | C | A |
| chr22 | 20065009 | rs415520 | C | G |
| chr22 | 20110836 | rs1640299 | T | G |
| chr22 | 20407094 | rs4020 | C | A |
| chr22 | 23315688 | rs440531 | A | C |
| chr22 | 23316029 | rs185140678 | C | A |
| chr22 | 23316030 | rs188387429 | T | G |
| chr22 | 24155941 | rs915595 | T | G |
| chr22 | 26464303 | rs2014410 | G | C |
| chr22 | 29306758 | rs2301585 | G | C |
| chr22 | 29306920 | rs2301586 | A | T |
| chr22 | 29307419 | rs9613859 | G | C |
| chr22 | 30654728 | rs757027 | C | A |
| chr22 | 30972110 | rs5749201 | A | T |
| chr22 | 31095309 | rs3205187 | G | C |
| chr22 | 31619464 | rs9956 | T | G |
| chr22 | 35346932 | rs743810 | T | G |
| chr22 | 38216365 | rs5995550 | A | C |
| chr22 | 38735722 | rs1043312 | T | G |
| chr22 | 39053498 | rs5750734 | G | T |
| chr22 | 41781449 | rs4822050 | G | C |
| chr22 | 41880738 | rs2228314 | G | C |
| chr22 | 42070505 | rs133375 | C | G |
| chr22 | 42079699 | rs2269524 | T | G |
| chr22 | 42869821 | rs7074 | G | T |
| chr22 | 44494965 | rs131154 | C | G |
| chr22 | 45134238 | rs7292511 | C | A |
| chr22 | 45327926 | rs11556482 | G | C |
| chr22 | 45340553 | rs1056322 | C | G |
| chr22 | 46684904 | rs1047123 | G | C |
| chr22 | 46685071 | rs801722 | T | G |
| chr22 | 46687115 | rs2748349 | T | A |
| chr22 | 49960624 | rs111752560 | A | C |
| chr22 | 50199168 | rs8238 | G | C |
| chr22 | 50343347 | rs72619589 | G | C |
| chr22 | 50549633 | rs140519 | G | T |
| chr22 | 50625611 | rs743616 | G | C |

### III.C Genotyping Snps

In some embodiments, one or more pre-determined SNPs include a genotyping SNP. Genotyping SNPs are SNPs associated with a particular sample or sample type and therefore can be used to differentiate samples.

In some embodiments, an allele is selected as a pre-determined SNP based, at least in part, on a SNPs ability to provide genotype information across samples (e.g., samples prepared with different assays).

Non-limiting examples of a pre-determined SNP that can be used as a genotyping SNP are provided in Table 3.

TABLE 3
Genotyping SNPs

| Chromosome | Position | rsid | ref | alt |
|---|---|---|---|---|
| chr1 | 634211 | rs560715817 | C | T |
| chr1 | 1310923 | rs41285824 | G | A |
| chr1 | 6221794 | rs1059867 | G | A |
| chr1 | 6599385 | rs2232461 | C | T |
| chr1 | 6599445 | rs2232460 | G | A |
| chr1 | 19312815 | rs2231192 | G | A |
| chr1 | 19312818 | rs139369121 | C | T |
| chr1 | 21247362 | rs1076669 | G | A |
| chr1 | 40861377 | rs72949149 | A | T |
| chr1 | 40861609 | rs1057635 | C | A |
| chr1 | 43338136 | rs17292650 | G | T |
| chr1 | 43338669 | rs12731981 | G | A |
| chr1 | 43704645 | rs304302 | G | A |
| chr1 | 43997532 | rs2286245 | C | T |
| chr1 | 46612965 | rs4660947 | T | C |
| chr1 | 52602421 | rs11205977 | G | T |
| chr1 | 52633413 | rs142476797 | C | T |
| chr1 | 89632759 | rs113690266 | G | A |
| chr1 | 92480739 | rs114464352 | T | C |
| chr1 | 1.01E+08 | rs3765684 | A | G |
| chr1 | 1.08E+08 | rs345269 | G | A |
| chr1 | 1.11E+08 | rs547905371 | T | G |
| chr1 | 1.55E+08 | rs35826120 | T | C |
| chr1 | 1.62E+08 | rs61803027 | T | C |
| chr1 | 1.62E+08 | rs34322334 | A | T |
| chr1 | 2.21E+08 | rs12141189 | T | C |
| chr1 | 2.27E+08 | rs74854864 | T | G |
| chr1 | 2.28E+08 | rs10916317 | A | G |
| chr1 | 2.36E+08 | rs6665008 | G | A |
| chr2 | 24492050 | rs535415536 | A | C |
| chr2 | 25246633 | rs2276598 | C | T |
| chr2 | 37671935 | rs12999211 | A | G |
| chr2 | 37672137 | rs13026016 | T | A |
| chr2 | 37672367 | rs114941880 | T | G |
| chr2 | 37672406 | rs56137036 | G | A |
| chr2 | 37672495 | rs17552689 | G | T |
| chr2 | 46297441 | rs17039192 | C | T |
| chr2 | 47790942 | rs1800932 | A | G |
| chr2 | 47800255 | rs56371757 | C | T |
| chr2 | 47803553 | rs2020910 | T | A |
| chr2 | 68319242 | rs4671898 | T | C |
| chr2 | 68319317 | rs13025842 | G | A |
| chr2 | 86790433 | rs79392961 | G | A |
| chr2 | 1.28E+08 | rs147371476 | C | A |
| chr2 | 1.58E+08 | rs3755401 | G | A |
| chr2 | 1.6E+08 | rs35284483 | A | G |
| chr2 | 1.66E+08 | rs111425435 | A | T |
| chr2 | 1.77E+08 | rs34744592 | A | G |
| chr2 | 1.81E+08 | rs113276800 | C | A |
| chr2 | 1.85E+08 | rs359895 | T | A |
| chr2 | 1.85E+08 | rs73041379 | G | A |
| chr2 | 2.08E+08 | rs11554137 | G | A |
| chr2 | 2.08E+08 | rs73070954 | C | T |
| chr2 | 2.18E+08 | rs2739048 | T | G |
| chr2 | 2.38E+08 | rs7240 | T | C |
| chr2 | 2.38E+08 | rs116000582 | A | G |
| chr2 | 2.38E+08 | rs3739061 | C | T |
| chr3 | 13325906 | rs665064 | C | T |
| chr3 | 18444681 | rs62240975 | G | A |
| chr3 | 23945356 | rs72627093 | A | T |
| chr3 | 37050534 | rs2020873 | C | T |
| chr3 | 45967999 | rs3796376 | C | T |
| chr3 | 45968128 | rs34147726 | C | T |
| chr3 | 45968489 | rs9875356 | C | T |
| chr3 | 45968515 | rs13071283 | T | C |
| chr3 | 63982224 | rs1053338 | A | G |
| chr3 | 1.14E+08 | rs3732799 | C | T |
| chr3 | 1.28E+08 | rs3087452 | T | G |
| chr3 | 1.3E+08 | rs7619850 | A | G |
| chr3 | 1.41E+08 | rs376975274 | C | T |
| chr3 | 1.43E+08 | rs6764683 | G | T |
| chr3 | 1.43E+08 | rs2280083 | G | A |
| chr3 | 1.43E+08 | rs4149494 | C | T |
| chr3 | 1.61E+08 | rs111314651 | T | C |
| chr3 | 1.61E+08 | rs533438138 | G | A |
| chr3 | 1.79E+08 | rs7611674 | T | G |
| chr3 | 1.84E+08 | rs148794859 | C | T |
| chr3 | 1.97E+08 | rs116984491 | G | A |
| chr4 | 56656054 | rs4626270 | A | G |
| chr4 | 56656229 | rs113431848 | G | A |
| chr4 | 85475380 | rs34267869 | C | T |
| chr4 | 85475529 | rs77314201 | T | C |
| chr4 | 1.05E+08 | rs76682196 | A | C |
| chr4 | 1.05E+08 | rs60786079 | G | A |
| chr4 | 1.4E+08 | rs72714251 | G | A |
| chr4 | 1.43E+08 | rs28989190 | C | T |
| chr4 | 1.53E+08 | rs184521106 | C | T |
| chr5 | 472836 | rs890974 | T | C |
| chr5 | 1064149 | rs143746308 | G | A |
| chr5 | 10564734 | rs814576 | C | T |
| chr5 | 98773768 | rs115735063 | C | T |
| chr5 | 1.43E+08 | rs10482609 | A | C |
| chr5 | 1.49E+08 | rs1801704 | C | T |
| chr5 | 1.49E+08 | rs1042713 | G | A |
| chr5 | 1.58E+08 | rs11465228 | C | T |
| chr6 | 13288303 | rs202040 | C | T |
| chr6 | 20212238 | rs12194843 | G | A |
| chr6 | 20212254 | rs148235151 | G | A |
| chr6 | 20212375 | rs113570493 | G | A |
| chr6 | 26522344 | rs116080308 | G | A |
| chr6 | 38170038 | rs3749926 | G | A |
| chr6 | 52362218 | rs75731219 | T | C |
| chr6 | 89433215 | rs138689380 | G | A |
| chr6 | 1.23E+08 | rs12523814 | C | T |
| chr6 | 1.47E+08 | rs144205394 | C | T |
| chr6 | 1.49E+08 | rs75156427 | G | A |
| chr6 | 1.49E+08 | rs79387518 | C | T |
| chr6 | 1.49E+08 | rs112722576 | G | A |
| chr6 | 1.52E+08 | rs17082422 | C | T |
| chr7 | 1459222 | rs61090716 | A | G |
| chr7 | 4762194 | rs61733617 | C | T |
| chr7 | 5593611 | rs187465308 | C | T |
| chr7 | 17298806 | rs7796976 | A | G |
| chr7 | 29684807 | rs191178315 | G | A |
| chr7 | 29685440 | rs116534988 | G | A |
| chr7 | 36153533 | rs66763009 | T | G |
| chr7 | 36153568 | rs140096401 | C | T |
| chr7 | 44885028 | rs6966024 | A | C |
| chr7 | 97117880 | rs62624461 | T | C |
| chr7 | 99558823 | rs6947941 | G | T |
| chr7 | 99558897 | rs6947826 | C | T |
| chr7 | 1.02E+08 | rs78058924 | C | A |
| chr7 | 1.02E+08 | rs75620414 | G | A |
| chr7 | 1.02E+08 | rs368214 | C | T |
| chr7 | 1.02E+08 | rs112726409 | G | A |
| chr7 | 1.02E+08 | rs142248299 | G | A |
| chr7 | 1.02E+08 | rs116434957 | A | G |
| chr7 | 1.02E+08 | rs56104629 | C | T |
| chr7 | 1.02E+08 | rs2529114 | G | T |
| chr7 | 1.02E+08 | rs35652575 | G | A |
| chr7 | 1.02E+08 | rs10259347 | A | G |
| chr7 | 1.02E+08 | rs2529115 | G | T |
| chr7 | 1.02E+08 | rs11771091 | G | A |
| chr7 | 1.02E+08 | rs73412055 | A | G |
| chr7 | 1.02E+08 | rs3087658 | G | A |
| chr7 | 1.02E+08 | rs113388724 | C | T |
| chr7 | 1.02E+08 | rs116793921 | A | C |
| chr7 | 1.02E+08 | rs813000 | G | A |
| chr7 | 1.02E+08 | rs2230103 | A | G |
| chr7 | 1.49E+08 | rs77051363 | A | G |
| chr8 | 23163833 | rs11135703 | G | A |
| chr8 | 27311137 | rs35188998 | A | G |
| chr8 | 60281082 | rs115885226 | T | C |
| chr8 | 1.18E+08 | rs76805972 | G | A |
| chr9 | 14314515 | rs73641905 | T | C |
| chr9 | 25677955 | rs34498078 | T | C |
| chr9 | 27529668 | rs77812016 | C | T |
| chr9 | 27529702 | rs3202600 | C | T |
| chr9 | 1.28E+08 | rs562125563 | T | G |
| chr9 | 1.28E+08 | rs35400405 | G | A |
| chr9 | 1.3E+08 | rs117436334 | G | A |
| chr9 | 1.31E+08 | rs116024762 | G | A |
| chr9 | 1.33E+08 | rs1050700 | C | T |
| chr9 | 1.37E+08 | rs3204123 | G | A |
| chr10 | 7161275 | rs9665413 | C | T |
| chr10 | 12349619 | rs145905575 | G | A |
| chr10 | 17453620 | rs45462798 | T | A |
| chr10 | 29735796 | rs34220528 | C | T |
| chr10 | 72088317 | rs2306324 | C | T |
| chr10 | 79315059 | rs3740259 | G | A |
| chr10 | 79315197 | rs45508000 | C | T |
| chr10 | 97714554 | rs139003280 | T | A |
| chr11 | 562437 | rs11246189 | G | A |
| chr11 | 2269820 | rs116549635 | G | A |
| chr11 | 61007755 | rs139918339 | C | T |
| chr11 | 61341502 | rs2260655 | G | A |
| chr11 | 61897520 | rs13966 | T | C |
| chr11 | 64357150 | rs61886888 | G | A |
| chr11 | 72013687 | rs35342866 | C | T |
| chr11 | 72015166 | rs3750912 | C | T |
| chr11 | 72721940 | rs11603334 | G | A |
| chr11 | 74254093 | rs17132881 | C | T |
| chr11 | 75768819 | rs7934862 | C | T |
| chr11 | 75769063 | rs35085051 | G | A |
| chr11 | 1.2E+08 | rs113799084 | C | T |
| chr11 | 1.23E+08 | rs147335078 | C | A |
| chr12 | 6384275 | rs41512347 | C | T |
| chr12 | 11891261 | rs1058028 | T | C |
| chr12 | 11892069 | rs72552356 | A | G |
| chr12 | 11893016 | rs11552161 | C | T |
| chr12 | 11894023 | rs76396773 | C | T |
| chr12 | 11894684 | rs1573613 | T | C |
| chr12 | 40224610 | rs1491945 | G | A |
| chr12 | 57759165 | rs1048691 | C | T |
| chr12 | 94149730 | rs2230754 | C | T |
| chr12 | 1.04E+08 | rs17041522 | C | T |
| chr12 | 1.17E+08 | rs118100421 | C | T |
| chr12 | 1.2E+08 | rs35490437 | C | T |
| chr12 | 1.2E+08 | rs7300790 | T | C |
| chr13 | 28061947 | rs7338903 | G | A |
| chr13 | 28718730 | rs1300234 | T | G |
| chr13 | 28718735 | rs3764098 | A | G |
| chr13 | 41193823 | rs140877303 | G | A |
| chr13 | 41458436 | rs7136 | T | C |
| chr14 | 23307872 | rs2231300 | G | A |
| chr14 | 23307890 | rs2231301 | G | A |
| chr14 | 72562901 | rs17780615 | C | T |
| chr14 | 72562999 | rs8020134 | T | C |
| chr14 | 1.03E+08 | rs34302315 | T | C |
| chr14 | 1.03E+08 | rs34174242 | G | A |
| chr14 | 1.04E+08 | rs74324704 | A | G |
| chr14 | 1.04E+08 | rs112809961 | T | C |
| chr15 | 43370561 | rs76609032 | T | A |
| chr15 | 43370631 | rs3809481 | G | A |
| chr15 | 79923673 | rs3803540 | C | A |
| chr15 | 83107504 | rs28444867 | C | A |
| chr15 | 83107874 | rs61323939 | C | A |
| chr15 | 84646233 | rs2271431 | T | G |
| chr16 | 297184 | rs214252 | A | G |
| chr16 | 1675036 | rs73499799 | C | T |
| chr16 | 1675296 | rs59823671 | C | T |
| chr16 | 14668001 | rs72789518 | C | T |
| chr16 | 31063854 | rs2303223 | G | A |
| chr16 | 56658083 | rs76144808 | G | T |
| chr16 | 84617682 | rs73257529 | C | A |
| chr16 | 89154280 | rs79800328 | C | T |
| chr17 | 4739441 | rs140340376 | G | A |
| chr17 | 7669124 | rs4968187 | C | T |
| chr17 | 10198392 | rs114822626 | G | A |
| chr17 | 31356976 | rs17881980 | C | T |
| chr17 | 40023239 | rs2302777 | A | G |
| chr17 | 43070958 | rs1799967 | C | T |
| chr17 | 44558348 | rs35283843 | T | C |
| chr17 | 56833691 | rs7219253 | C | T |
| chr17 | 60656730 | rs111239559 | A | C |
| chr17 | 60665826 | rs116005345 | C | T |
| chr17 | 74212952 | rs60217659 | C | A |
| chr17 | 75093611 | rs4789134 | G | A |
| chr17 | 75093757 | rs4788863 | T | C |
| chr17 | 75627122 | rs74528906 | T | C |
| chr17 | 78138595 | rs142857824 | C | T |
| chr17 | 78141720 | rs11651404 | T | A |
| chr17 | 78141852 | rs11654773 | T | G |
| chr17 | 80109992 | rs1800305 | C | T |
| chr17 | 80415678 | rs35549084 | G | A |
| chr17 | 81252464 | rs35546507 | T | C |
| chr18 | 3450206 | rs7233448 | A | T |
| chr18 | 62524195 | rs7229802 | G | A |
| chr19 | 5915381 | rs10423464 | T | C |
| chr19 | 10252575 | rs113197610 | A | C |
| chr19 | 10514059 | rs35483143 | A | T |
| chr19 | 10514445 | rs34803021 | G | A |
| chr19 | 12885686 | rs2072596 | A | G |
| chr19 | 12885905 | rs117351327 | G | A |
| chr19 | 12885926 | rs2072597 | A | G |
| chr19 | 17539244 | rs74546231 | G | A |
| chr19 | 17539420 | rs114207587 | C | A |
| chr19 | 19004193 | rs10409265 | T | C |
| chr19 | 19626877 | rs33982830 | C | T |
| chr19 | 33300901 | rs1049969 | T | C |
| chr19 | 33301036 | rs4142943 | G | A |
| chr19 | 33301842 | rs192240793 | G | A |
| chr19 | 33622277 | rs191155315 | G | T |
| chr19 | 34963700 | rs2546028 | A | C |
| chr19 | 34963866 | rs111702221 | C | T |
| chr19 | 45145245 | rs10419874 | A | G |
| chr19 | 47725912 | rs8111184 | A | G |
| chr19 | 49809544 | rs35002951 | C | T |
| chr19 | 50725377 | rs11084024 | G | A |
| chr19 | 56368398 | rs142343375 | G | A |
| chr19 | 57614276 | rs2269818 | A | G |
| chr19 | 58326896 | rs113019525 | C | T |
| chr19 | 58499157 | rs77807864 | C | T |
| chr20 | 31605735 | rs15817 | A | G |
| chr20 | 32434666 | rs3746609 | G | A |
| chr20 | 32434962 | rs35712951 | C | T |
| chr20 | 32435225 | rs35632616 | A | G |
| chr20 | 32435697 | rs62206933 | C | T |
| chr20 | 32436685 | rs6057581 | C | T |
| chr20 | 32437732 | rs2295762 | A | G |
| chr20 | 32437764 | rs55820705 | T | C |
| chr20 | 32438576 | rs142200477 | C | T |
| chr20 | 38146733 | rs2294545 | G | A |
| chr20 | 47736827 | rs3810526 | A | G |
| chr20 | 63863966 | rs74432425 | G | A |
| chr20 | 63864109 | rs3795149 | G | A |
| chr20 | 63864135 | rs77107743 | T | G |
| chr20 | 64048520 | rs183578654 | C | T |
| chr21 | 34788103 | rs78335539 | A | G |
| chr21 | 34789075 | rs76478380 | A | G |
| chr21 | 34790997 | rs55744508 | G | T |
| chr21 | 34791123 | rs55767668 | G | A |
| chr21 | 34792047 | rs539980908 | C | A |
| chr21 | 34792065 | rs150481777 | A | G |
| chr21 | 34792108 | rs13051066 | G | T |
| chr21 | 34799341 | rs59802347 | G | A |
| chr21 | 34887027 | rs111527738 | A | G |
| chr21 | 43762120 | rs1300 | T | C |
| chr21 | 44329577 | rs115857899 | C | T |
| chr21 | 45530949 | rs79091853 | C | T |
| chr22 | 22888120 | rs382768 | C | T |
| chr22 | 23180952 | rs139121414 | G | A |
| chr22 | 29695776 | rs8140096 | C | T |
| chr22 | 41688233 | rs73161344 | T | C |
| chr22 | 49903558 | rs116765369 | C | T |
| chr22 | 49903598 | rs76848348 | C | T |
| chr22 | 50248072 | rs36039258 | A | T |
| chr22 | 50439767 | rs13057311 | G | A |

### IV. Analytical Validation to Determine Limit of Detection for Methods Using Pre-Determined Snps

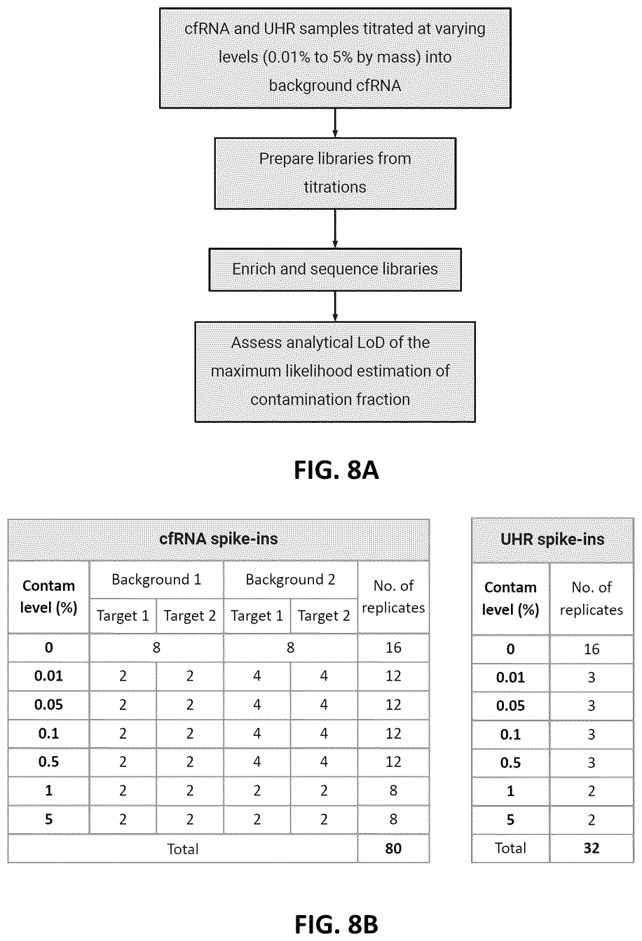

To determine the limit of detection (LOD) of contamination detection workflow 600, different contamination levels of cfRNA (“cfRNA spike-ins”) and UHR (“UHR spike-ins”) ranging from 5% down to 0.01% by mass (see, e.g., FIGS. 8A-8B) were mixed into background cfRNA. Limit of detection was assessed using maximum likelihood estimation of contamination fraction (i.e., at step 620 in FIG. 6 a maximum likelihood estimation was used). Here, the limit of detection is considered to be the lowest contamination level at which the specificity is above 95%.

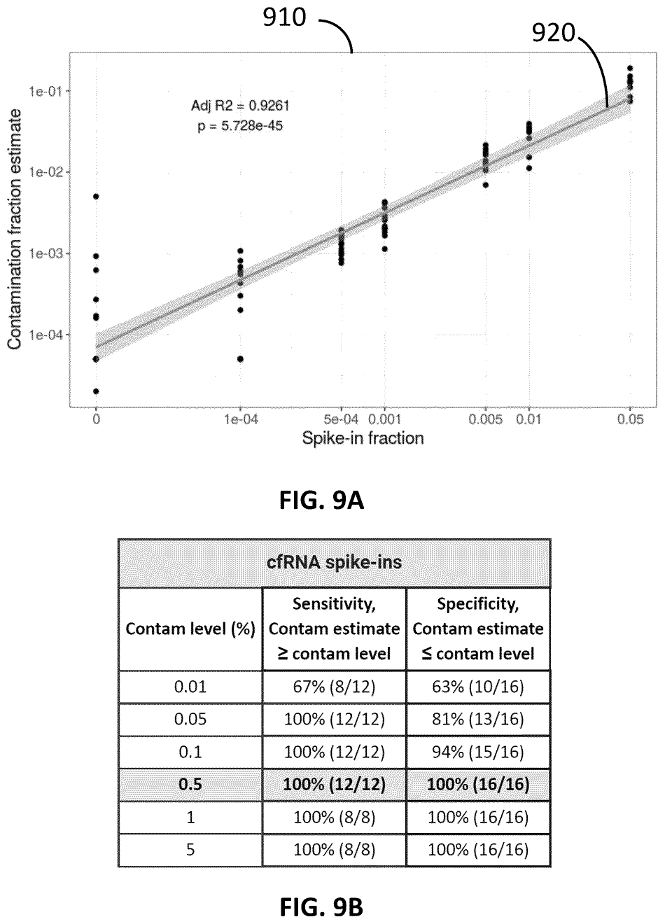

FIG. 9A is a plot showing the analytical validation for limit of detection for cfRNA contamination using the detection methods described herein. Plot 910 shows a best fit line 920 of the detection rate obtained at each cfFNA spike-in level (see, e.g., FIG. 9A numeral 920 having Adj R²=0.9261, p=5.728e-45). FIG. 9B shows limit of detection of cfRNA spike-ins using detection workflow 600 (and as shown in FIG. 8A) was 0.5% contamination level.

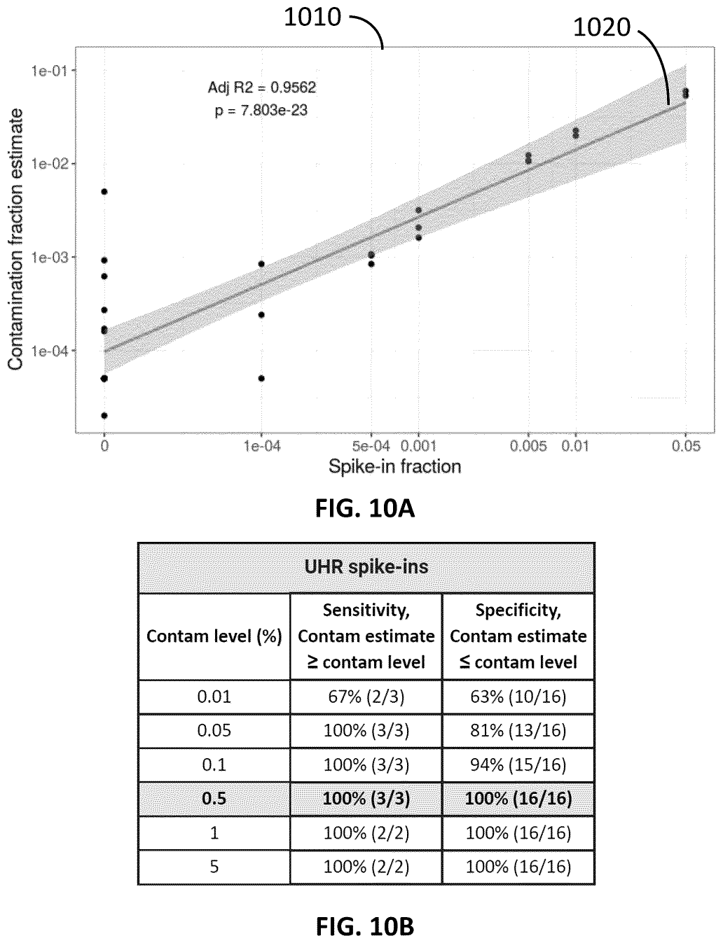

FIG. 10A is a plot showing the analytical validation for limit of detection of UHR contamination using the detection methods described herein. Plot 1010 shows a best fit line 1020 of the detection rate obtained at each UHR spike-in level (see, e.g., FIG. 10A numeral 1020 having Adj R²=0.9562, p=7.803e-23). FIG. 10B shows limit of detection of UHR spike-ins using detection workflow 600 (and as shown in FIG. 8A) was 0.5% contamination level.

Limit of detection for detection workflow 600 (e.g., Step 620) can also be measured using a robust linear regression model for contamination detection (see, e.g., PCT/IB2018/050979, which is incorporated herein by reference in its entirety).

### V. Validation of Contamination Detection Using Pre-Determined Snps and Likelihood Tests

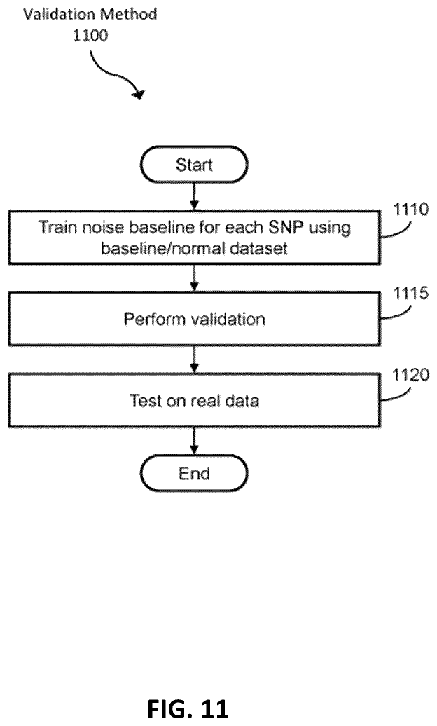

Detection workflow 600 using maximum likelihood estimation for contamination probability determinations (i.e., at step 620 in FIG. 6 a maximum likelihood estimation was used) was validated using a three-step process. FIG. 11 illustrates an example of a method 1100 for validating contamination detection workflow (e.g., workflow 600 or 700). Validation method 1100 may include, but is not limited to, the following steps.

At a step 1100, a background noise baseline for each SNP is generated using a set of normal training samples (e.g., 80 normal, uncontaminated samples). The noise baseline provides an estimate of the expected noise for each SNP and is used to distinguish a contamination event from a background noise signal. Generation of a noise (contamination) baseline is described in more detail in PCT/US2018/039609, which is incorporated herein by reference in its entirety.

At a step 1115, a 5-fold cross-validation process is performed. For example, datasets of 24 normal samples and in silico titrations are partitioned into a validation set and a training set. Here, the contamination levels ranges from 0.05% to 50%. The training set is used to train detection method 600 and set a threshold for calling a contamination event versus normal background noise. That is, detection method 600 can include a different threshold for each threshold and repeat of an SNP. The threshold is then tested on the validation set. This process is repeated a total of 10 times to identify a final threshold and LOD for calling a contamination event.

At a step 1120, the final threshold and LOD are tested on a real dataset (e.g., a cfDNA dataset from cancer patient samples).

FIGS. 12A-D show a workflow (FIG. 12A) and a plot (FIG. 12B) showing preliminary in silico validation of the detection method workflow 600 using whole transcriptome data of plasma from two individuals titrated with background plasma at 0%, 0.01%, 0.05%, 0.1%, 0.5%, 1% and 5%. Observed allele frequencies were determined for sequencing reads identified as having one or more pre-determined single nucleotide polymorphisms (SNPs). Contamination probability was determined using maximum likelihood estimation using the methods described herein and described in PCT/US2018/039609, which is incorporated herein by reference in its entirety.

FIG. 12C and FIG. 12D shows that contamination fraction estimates with small panels correlate better with average log likelihood (predicting the presence of contamination in a sample) than the same correlation calculation when analyzing SNPs from whole transcriptome data.

### VI. Detecting Contamination Using-Likelihood Tests

In one embodiment, a method for identifying contamination in a sample includes applying at least one likelihood test (i.e., a contamination model) to the sequencing reads. In one embodiment, a method for identifying contamination in a sample includes applying at least one likelihood test (i.e., a contamination model) to the sequencing reads identified as having one or more pre-determined SNPs and an observed allele frequency in the plurality of sequencing reads. Exemplary methods for using likelihood tests for contamination detection are described in PCT/US2018/039609, which is incorporated herein by reference in its entirety.

In some embodiments, one or more likelihood tests are applied to a sequencing read of the plurality of sequencing reads using the associated contamination probability. In such cases, each likelihood test is used to obtain a current contamination probability is indicative of whether the sequencing reads are contaminated. In one embodiment, each likelihood test is used to obtain a confidence score representing a measure of the predicted contamination in the sequencing reads.

In one embodiment, a method of identifying contamination in a sample that includes applying at least one likelihood test (e.g., a contamination model) further includes a step of determining that the sequencing reads are contaminated based on the current contamination probability of the at least one test being above a threshold associated with the at least one test likelihood test.

In one embodiment, a method of identifying contamination in a sample that includes applying at least one likelihood test (e.g., a contamination model) further includes a step of determining that the sequencing reads are contaminated based on the current contamination probability of at least two likelihood tests being above a threshold associated with the at least two likelihood tests. In such cases, the threshold for each likelihood test can be the same. In other cases, the threshold for each likelihood test can be different.

In one embodiment, the at least one likelihood test maximizes a likelihood function, the likelihood function proportional to the probability of an event occurring in a data set given a variable.

In one embodiment, applying the at least one likelihood test of the contamination model comprises: comparing a set of generated contaminated sequencing reads to a set of previously obtained non-contaminated sequencing reads to determine the contamination probability.

In one embodiment, applying at least one likelihood test of the contamination model comprises: generating a null hypothesis representing that the sequencing reads are not contaminated; generating a set of contamination hypotheses representing that the sequencing reads are contaminated, wherein each contamination hypothesis of the set of contamination hypotheses is contaminated at a different contamination level; applying a likelihood ratio test between the set of contamination hypotheses and the null hypothesis, the likelihood ratio test to obtain the current contamination probability.

In one embodiment, applying the at least one likelihood test of the contamination model comprises: comparing a set of generated contaminated sequencing reads to an average of previously obtained sequencing reads to determine the contamination probability, the contamination probability associated with the likelihood that the sequencing reads are contaminated at a contamination level.

In one embodiment, applying at least one likelihood test of the contamination model comprises: generating a set of contamination hypotheses representing that the sequencing reads are contaminated, wherein each contamination hypothesis of the set of contamination hypotheses is contaminated at a different contamination level; generating a null hypothesis representing the mean minor allele frequency at a contamination level for a plurality of previously obtained sequencing reads, wherein the contamination level is associated with the contamination hypothesis most likely to be contaminated; and applying a likelihood ratio test between the set of contamination hypotheses and the null hypothesis, the likelihood ratio test to obtain the current contamination probability.

In some embodiments, it is important to be able to distinguish between contamination and noise. As noted above, processing system 200 can be used to detect contamination in a test sample. For example, using the contamination detection workflow 700 a contamination event can be detected based on a plurality (or set) of observed variant allele frequencies in a test sample. In one embodiment, the observed variant allele frequencies can be compared to population MAFs from a plurality of SNPs for the detection of cross-sample contamination.

In a non-limiting example, FIG. 7 illustrates a flow diagram illustrating a contamination detection workflow 700. The detection workflow 700 of this embodiment includes, but is not limited to, the following steps.

At step 710, sequencing data obtained from a sample (e.g., using the process 300) is cleaned up. In some embodiments, data cleaning may include removing a pre-determined SNPs with no-calls (e.g., no coverage), a sequencing depth less than a threshold (e.g., any of the sequence depth thresholds described herein), high error frequencies (e.g., >0.1%), high variance, and/or low coverage. In other examples, homozygous alternative SNPs with variant frequency 0.8 to 1.0 can be negated (e.g., variant frequency 0.95 becomes 0.05) in order to put all the variant frequency data in one scale that can be linearly compared to minor allele frequency values. Further, the MAF values can be negated based on a samples genotype.

At step 715, optionally, observed allele frequencies for each of the one or more pre-determined SNPs is determined.

At step 717, optionally, a contamination probability for each pre-determined SNP is determined using the observed allele frequency for each pre-determined SNP. In one example, a prior probability of contamination is calculated for each SNP based on host sample's genotype and minor allele frequency.

At step 720, a likelihood model including a maximum likelihood estimation is applied to determine contamination based on the probability of contamination for the pre-determined SNPs. The likelihood model includes a first and a second likelihood test as described herein.

At a decision step 725, it is determined whether the test sample is contaminated. If a test sample passes both likelihood tests, then the sample is contaminated and workflow 700 proceeds to a step 730. If a test sample does not pass both likelihood tests, then the workflow is not contaminated and workflow 700 ends.

At step 730, a likely source of contamination is identified based on the prior probabilities of SNPs from known genotypes of other samples that were processed in the same batch as the sample (or a set of related batches).

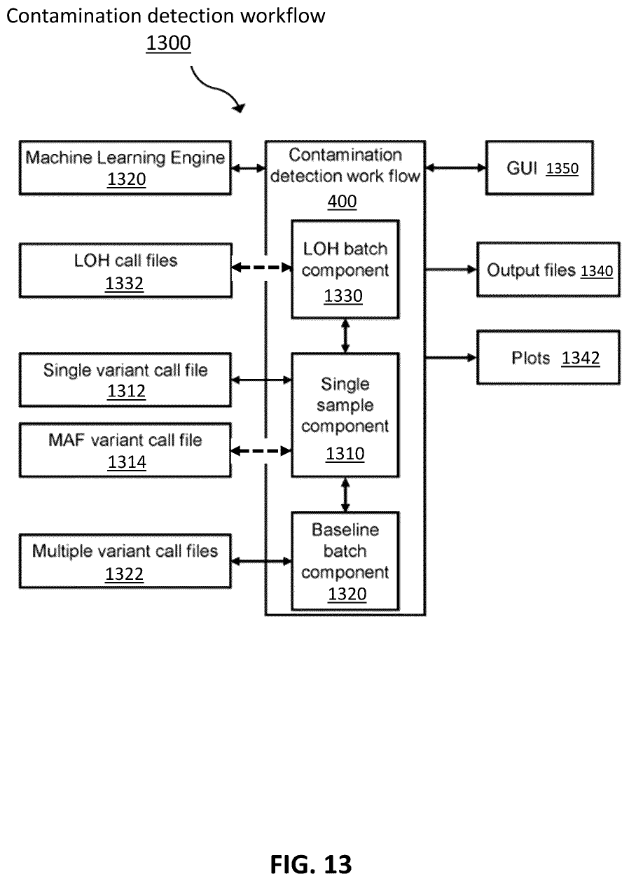

In one embodiment, method 700 is executed according to workflow 1300. For example, FIG. 13 provides a diagram of a contamination detection workflow 1300 executing on the processing system 200 for detecting and calling contamination, in accordance with applying at least one likelihood test (i.e., a contamination model).

In the illustrated example, contamination detection workflow 1300 includes a single sample component 1310, a baseline batch component 1320, and an optional loss of heterozygosity (LOH) batch component 1330. Single sample component 1310 of contamination detection workflow 1300 is informed, for example, by the contents of a single variant call file 1312 and a minor allele frequencies (MAF) variant call file 1314 called by the variant caller 240. The single variant call file 1312 is the variant call file for a single target sample. The MAF variant call file 1314 is the MAF variant call file for any number of SNP population allele frequencies AF.

Baseline batch component 1320 of contamination detection workflow 1300 generates a background noise baseline for each SNP from uncontaminated samples as another input to single sample component 1310. Generating a background noise baseline using a contamination noise baseline workflow is described in more detail in regard to FIG. 13. Baseline batch component 1320 is informed, for example, by the contents of multiple variant call files 1322 called by the variant caller 240. The multiple variant call files 1322 can be the variant call files of multiple samples.

LOH batch component 1330 of contamination detection workflow 1300 determines a LOH in samples as another input to the single sample component 1310. LOH batch component 1330 is informed, for example, by the contents of LOH call files 1332. The LOH call files are call files for a plurality of alleles previously determined to include SNPs with LOH in the sample. The LOH call files can be called by the variant caller 240 and stored in the sequence database 210.

In one embodiment, the contamination detection workflow 1300 can generate output files 1340 and/or plots 1342 from sequencing data processed by contamination detection algorithm 110. For example, contamination detection workflow 1300 may generate log-likelihood data and/or display log-likelihood plots 1342 as a means for evaluating a DNA test sample for contamination. Data processed by contamination detection workflow 1300 can be visually presented to the user via a graphical user interface (GUI) 1350 of the processing system 200. For example, the contents of output files 1340 (e.g., a text file of data opened in Excel) and log-likelihood plots 1342 can be displayed in GUI 1350.

In another embodiment, the contamination detection workflow 1300 may use the machine learning engine 220 to improve contamination detection. Various training datasets (e.g., parameters from parameter database 230, sequences from sequence database 210, etc.) may be used to supply information to the machine learning engine 220 as described herein. In accordance with this embodiment, the machine learning engine 220 may be used to train a contamination noise baseline to identify a noise threshold, detect loss of heterozygosity, and determine the limit of detection (LOD) for contamination detection.

Single sample component 1310 of contamination detection workflow 1300 is, for example, a runnable script that is used to estimate contamination in a sample. By contrast, baseline batch component 1330 of contamination detection algorithm 110 is, for example, a runnable script that is used for generating estimates across a batch of samples, and may also be used to generate the noise model across these samples (if the input batch is healthy). Similarly, LOH batch component 1330 of contamination detection model is, for example, a runnable script that is used for generating estimates across a batch of samples, and may be used to determine the LOH in single samples based on the generated estimates.

In one embodiment, the contamination detection workflow 1300 may be based on a model for estimating contamination. In one embodiment, the model is a maximum likelihood model (herein referred to as the likelihood model) for detecting contamination in sequencing data from a sample. However, in other examples, the model can be any other estimation model such as an M-estimator, maximum spacing estimation, method of support, etc.

In one example, the likelihood model determines contamination by calculating the probability of observing a MAF of a sample at a given contamination level a and, subsequently, determining if the sample is contaminated. In some embodiments, the likelihood model is informed by prior probabilities of contamination that are first calculated for each pre-determined SNP in the sample based on the genotype of previously observed contaminated samples.

Further, the contamination detection workflow 1300 can, in some cases, determine the likely source of contamination for the observed sample. That is, the likelihood model can compare sequencing data from several contaminated samples to determine a source of contamination. The likelihood model can be informed by prior probabilities of contamination from other samples with a known genotype to identify a likely source of contamination. In some embodiments, genotype is determined by identifying sequencing reads have a pre-determined genotyping SNP.

### VI.A Probability of Contamination for a Single Pre-Determined SNP

The contamination detection workflow 1300 determines a probability that a sample is contaminated using prior probabilities and observed sequencing data (FIG. 13). In some examples, the observed sequencing data can be included in a sample call file (such as single variant call file 1312), optionally a LOH call file (such as LOH call file 1332), and optionally a population call file (such as MAF call file 1314). The prior probabilities of contamination can be determined based on the observed sequencing data. Here, for purpose of example, the probability of contamination for a single pre-determined SNP is based on a samples minor allele frequency MAF and the error rate of previously observed homozygous SNPs. In some embodiments, the contamination detection workflow 1300 can additionally or alternatively use, for example, alternate allele frequency, noise rates, and read depths to determine a contamination probability.

Contamination detection workflow 1300 compares the probability of observing data in the plurality of sequencing reads using two different models. In one model, there is no contamination and any sequencing reads with alternative alleles at the site are either the result of noise in the plurality of sequencing reads or of heterozygosity of the plurality of sequencing reads at a site of a pre-determined SNP. In the other model, there is contamination of the sample and sequencing reads with alternative alleles can be the result of correctly reading a contaminating cfRNA strand. In this context, contamination detection workflow 1300 calculates a ratio between the likelihood the sample is contaminated and the likelihood the sample is uncontaminated using the two models. Based on the ratio, contamination detection workflow can determine if the sample is contaminated or uncontaminated.

In one embodiment, the probability of contamination at a single pre-determined SNP site for a given set of data D is calculated as:

$$P ( α \vert D ) = P ( α ) \cdot P ( α ) \qquad ( 1 )$$

where P(α|D) is the probability of observing the contamination level alpha given the data D, P(D|α) is the probability of observing the data given the contamination level alpha, and P(α) is the probability of the contamination level alpha. Therefore, in an example where there is no contamination in the sample, the probability of contamination in a sample can be represented as:

$$P ( α = 0 \vert D ) = P ( α = 0 ) \cdot P ( α = 0 ) \qquad ( 2 )$$

where a=0 indicates that the contamination level a is 0.0%.

In one embodiment, in samples where the contamination level is non-zero, the probability of observing data D with a contamination level a for a given set of data D (P(D|α)) is further based on the genotype of the contaminant G_{C} and the genotype of the host GH (the source of the test sample). That is, the probability of observing data D given a contamination level a can be represented as:

$$P ( α ) = {\sum }_{{G}_{H} , {G}_{C}} P ( {G}_{H} ) \cdot P ( {G}_{C} ) \cdot P ( D \vert p ) \qquad ( 3 )$$

where P(G_{C}) is the probability that the contamination at the pre-determined SNP site will be the type associated with the genotype of the contaminant at that site, P(G_{H}) is the probability that the contamination at the site will be the genotype of the host at that site, and P(D|p) is the probability of observing the data D given a set of characteristics p. Here, the set of characteristics p include the probability of an SNP mutation & for the pre-determined SNP site and the contamination level a but can include any other characteristics of the sample. The summation over the genotypes indicates that the probability of observing data at a contamination level a includes contributions based on the three possible genotypes of the contaminant and host (A/A, A/B, and B/B).

For a given pre-determined SNP the probability of observing the data at a given contamination level alpha can be represented with a generic site specific model. The generic site specific model can be represented as:

$$P ( α ) = P ( {AA}_{host} ) \cdot P ( {AA}_{cont} ) \cdot P ( p = ε ) + P ( {AA}_{host} ) \cdot P ( {AB}_{cont} ) \cdot P ( p = ε + \frac{a}{2} ) + P ( {AA}_{host} ) \cdot P ( {BB}_{cont} ) \cdot P ( p = ε + α ) + … P ( {BB}_{host} ) \cdot P ( {BB}_{cont} ) \cdot P ( p = ε ) \qquad ( 4 )$$

where AA is a homozygous reference allele, AB is a heterozygous allele, BB is a homozygous alternative allele, the subscript “host” represents the genotype of the host G_{H}, the subscript “cont” represents the genotype of the contaminant, & is the probability of observing a specific mutation, and α is the contamination level.

In some cases, the generic site specific model can be modeled with a binomial distribution. For example, for a specific case from the generic site specific model, the probability of observing the data D at a given contamination level alpha can be represented as:

$$P ( α ) = P ( A {A}_{host} , {AB}_{cont} , α ) = binomial ( DP , MAD , \frac{α}{2} + ε ) \qquad ( 5 )$$

where “binomial” is the binomial probability of observing the data based on depth DP and minor allele depth MAD (minor allele depth) of the test sample, the genotype of the host (A/A), the genotype of the contaminant (A/B), the contamination level a, and the probability of observing a specific error or mutation ¿.

The generic site specific model can be simplified using prior probabilities of contamination. The simplified model can be represented as:

$$P ( α ) = {P}_{C} \cdot P ( α , C ) + ( 1 - {P}_{C} ) P ( α = 0 , ! C ) \qquad ( 6 )$$

where P_{C} is the probability of contamination of the sample based on a prior observation of a contaminant with a genotype different from the host genotype C, P(D|α,C) is the probability of observing the data D with a contamination level a given the SNP is contaminated, (1-P_{c}) is the probability of no contamination and P(D|α=0,!C) is the probability of observing data D with a contamination level a of 0% (i.e., no contamination, denoted as!C).

Alternatively stated, P_{C} is the probability that an SNP at a site is contaminated with a contaminant of a different allele type than the host given a contamination level α. In one example, the simplified model determines the prior probability of contamination P_{C} using the following:

$${P}_{C} = \{ 1 - {( 1 - M A F )}^{2} 1 - M A {F}^{2} if host A / A if host B / B$$

where MAF is the minor allele frequency, A/A is a homozygous reference allele, and B/B is a homozygous alternative allele. Here, heterozygous alleles are removed and are not considered in determining the probability of contamination for a sample.

### VI.B Probability of Contamination for a Sample

As previously described, in one embodiment, the contamination detection workflow 1300 uses a likelihood model to determine contamination in a sample. Here, to determine contamination in a sample, the likelihood model determines a level of contamination a that maximizes a likelihood function L(α). The likelihood function L(α) can be written as:

$$L ( α ) ∝ P ( α ) = {\prod }_{i = 1}^{N} max ( P ( α ) , β ) \qquad ( 7 )$$

where P(D|α) is the probability of observing data D given contamination level α, β is a minimum allowable probability, N is the number of homozygous (A\A or B\B) SNPs of the sample, and Di is the observed data for a given pre-determined SNP.

The likelihood function L(α) is proportional to the probability of observing data D given a contamination level α(P(D|α)). The probability of the data D given a contamination level α takes into account all pre-determined SNPs of the sample. That is, L(α) is the product over each pre-determined SNP in the sample of the maximum of the probability of the data in that pre-determined SNP given the contamination level α(P(Dᵢ|α)). For each pre-determined SNP, if the probability of the data D given a contamination level α is below a threshold, the probability for that pre-determined SNP can be assigned a value β. The value β is a minimum probability that is set as a black swan term (e.g., β=3.3×10⁻⁷) which limits the lowest value each pre-determined SNP evaluated can contribute to the likelihood function L(α). The probability of contamination at of a single pre-determined SNP site (P(Dᵢ|α)) is described in more detail in Section V.A.

### VI.C Probability of Contamination for a Sample Using Likelihood Tests

In one example of determining the likelihood of contamination, the contamination detection workflow 1300 applies a likelihood model including two separate likelihoods tests.

In the first likelihood test, the product term of the likelihood function L(α) is used to calculate a first likelihood ratio (LR) representing the maximum contamination likelihood that is obtained from testing a series of contamination levels ai against the minor allele frequency in a sample. That is, which level of contamination a gives the highest contamination likelihood.

The first likelihood ratio LR₁ uses a first null hypothesis that the sample is contaminated at a maximum of a series of contamination levels a (L(α=ai)) based on the MAF of the observed, pre-determined SNPs. That is, the sample is contaminated at a contamination level Qmax giving the highest likelihood of contamination. Therefore, the first null hypothesis can be written as:

$${L}_{max} = max [ {L}_{1} ( α = . 001 ) , {L}_{2} ( α = . 002 ) , … {L}_{i} \cdot ( α = . 5 ) ] \qquad ( 8 )$$

The first likelihood ratio also uses a first hypothesis that there is no contamination in the sample (L(α=0.000)). Therefore, the first likelihood ratio test LR₁ can be written as:

$$L {R}_{1} = \frac{max [ L ( α = 0.001 ) , L ( α = 0.002 ) , L ( α = 0.003 ) … L ( α = \text{.5} ) ]}{L ( α = 0. )} \qquad ( 9 )$$

Generally, the first likelihood ratio LR₁ results in a value. The sample is considered to pass the first likelihood test if the value of the first likelihood ratio LR₁ is above a threshold level. That is, it is likely that the sample is contaminated at a contamination level α.

In the second likelihood test, the likelihood function L(α) is used to calculate a second likelihood ratio LR₂ representing a likelihood that observed minor allele frequencies are due to contamination rather than due to a constant increase in noise across all pre-determined SNPs or all SNPs.

The second likelihood ratio LR₂ uses a second null hypothesis Lₘₐₓ MAF that is the same as the first null hypotheses (Eqn. 4). Additionally, the second likelihood ratio LR₂ uses a second hypothesis Lnoise that a sample contaminated at contamination level amax includes minor allele frequencies at an average allele frequency of previously observed SNPs (e.g., pre-determined SNPs or all SNPs) (uniform (MAF)). The second null hypothesis can be written as:

$${L}_{n o i s e} = L ( {α}_{max} \vert uniform ( MAF ) ) \qquad ( 10 )$$

Accordingly, the second likelihood ratio can be written as:

$$L {R}_{2} = \frac{{L}_{max}}{{L}_{n o i s e}} = \frac{max [ {L}_{1} ( a = 0.001 ) , {L}_{2} ( α = 0.002 ) , … {L}_{i} ( α = \text{.5} )}{L ( {α}_{max} \vert u niform ( mAF ) )} \qquad ( 11 )$$

The second likelihood ratio LR₂ results in a value. The sample is considered to pass the second likelihood test LR₂ if the value is above a threshold. That is, it is likely that the observed MAF is due to contamination and not due to noise. Alternatively stated, the second likelihood test passes when a specific arrangement of previously observed MAFs are significant in determining the contamination likelihood, while a random distribution of previously observed MAFs are insignificant in determining contamination likelihood.

If a sample passes both of the likelihood tests, then the sample is called as contaminated at contamination level α which passes the tests. If a sample fails either of the likelihood tests, then it is not called as contaminated.

In other configurations, the contamination detection workflow can use additional or fewer likelihood tests to determine if a sample is contaminated.

### VI.D Determining a Contamination Source

In one example of determining the likelihood of contamination, the likelihood model of the contamination detection workflow 400 can additionally determine a likely source of contamination. Detecting the source of contamination enables the assessment of risk introduced by the contaminant, as well as the point in sample process in which it happened, such as, for example, any step of process 100 or 300. In contamination detection workflow 600 or 700, the genotypes of likely contaminants may be used in place of prior probabilities from population SNPs. Introduction of prior probabilities of contamination will either increase or decrease the likelihood ratio relative to the likelihood ratio obtained by for probabilities based on the population.

The likelihood model can be informed by the prior probabilities of pre-determined SNPs from the known genotypes of samples that were processed in the same batch as the test sample (or a set of related batches). A likelihood test is then performed to determine if knowing the exact genotype probabilities gives a higher value than the likelihood obtained using the population MAF probability. If the difference is significant, it can be concluded that a given sample is the contaminant.

For a given pre-determined SNP, three observed genotypes are possible: homozygous reference 0/0, heterozygous 0/1, and homozygous alternative 1/1, where 0 represents the reference allele and 1 the alternative allele. In a normal (uncontaminated) sample, the expected allele frequency values observed are expected to be close to 0, 0.5 and 1 for genotypes 0/0, 0/1 and 1/1, respectively. However, in a contaminated sample, the observed allele frequency values can be expected to shift from 0, 0.5, and 1, as the pre-determined SNPs vary across the population, and thus, have a higher likelihood of being present in a contaminating sample.

### VII. Detecting Contamination Using-Regression

In one embodiment, a method for identifying contamination in a sample includes generating a noise model (i.e., a contamination model) based on the sequencing reads. In one embodiment, a method for identifying contamination in a sample includes generating a noise model (i.e., a contamination model) based on the sequencing reads identified as having one or more pre-determined SNPs and an observed allele frequency in the plurality of sequencing reads. Exemplary methods for using regression analysis for contamination detection are described in PCT/IB2018/050979, which is incorporated herein by reference in its entirety.

In one embodiment, the noise model represents a measure of background noise in a subset of sequencing reads, the noise model generated based on the subset of the sequencing reads. The background noise can be a population measure of allele frequency in the subset of sequencing reads. Additionally, the background noise can be representative of the static noise generated when sequencing a SNP.

In one embodiment, a method of identifying contamination in a sample that includes applying a noise model (e.g., a contamination model) further includes applying the contamination model to an identified sequencing read using the observed allele frequency of the one or more pre-determined SNPs in the identified sequencing reads and the generated noise model to obtain a confidence score representing a measure of the predicted contamination in the sequencing reads. In such cases, a plurality of sequencing reads (e.g., a sample) is identified as contaminated when the confidence score is above a threshold that the contamination model predicts is indicative of contamination. Contamination models can include a random error term to aid in generating a confidence score.

In one embodiment, generating the noise model further comprises: determining a noise coefficient for each SNP of the subset of sequencing reads, the noise coefficient predicting the expected noise level for each SNP. In some embodiments, the noise model generated based on the subset of sequencing reads is additionally based on a sample type of the sequencing reads.

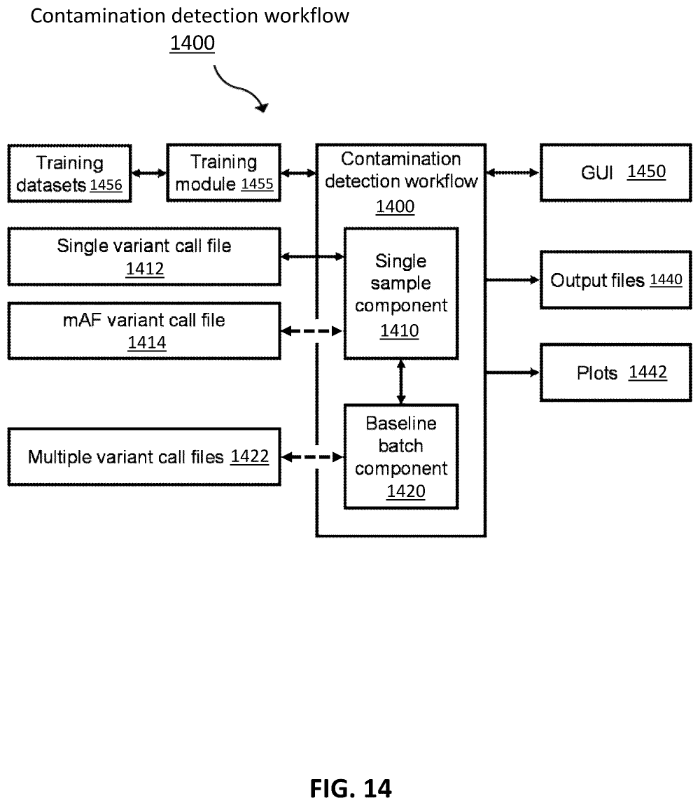

In a non-limiting example, FIG. 14 provides a diagram of a contamination detection workflow 1400 executing on the processing system 200 for detecting and calling contamination, applying a noise model (i.e., a contamination model).

In the illustrated example, contamination detection workflow 1400 includes a single sample component 1410 and a baseline batch component 1420. Single sample component 1410 of contamination detection workflow 1400 is informed, for example, by the contents of a single variant call file 1412 and a minor allele frequencies (MAF) variant call file 1414 called by the variant caller 240. The single variant call file 1412 is the variant call file for a single target sample. The MAF variant call file 1414 is the MAF variant call file for any number of SNP population allele frequencies AF.

Baseline batch component 1420 of contamination detection workflow 1400 generates a background noise baseline for each SNP from uncontaminated samples as another input to the single sample component 1410. Generating a background noise baseline is described in more detail below. Baseline batch component 1420 is informed, for example, by the contents of multiple variant call files 1422 called by the variant caller 240. The multiple variant call files 1422 can be the variant call files of multiple samples and are, in some examples, variants that are determined to be healthy samples. Healthy samples are samples previously determined not to include cancer.

In one embodiment, the contamination detection workflow 1400 can generate output files 1440 and/or plots 1442 from sequencing data processed by contamination detection algorithm 110. For example, contamination detection workflow 1400 may generate variant allele frequency distribution plots or regression plots as a means for evaluating a DNA test sample for contamination. Data processed by contamination detection workflow 1400 can be visually presented to the user via a graphical user interface (GUI) 1450 of the processing system 200. For example, the contents of output files 1440 (e.g., a text file of data opened in Excel) and regression plots 1442, for example, can be displayed in GUI 1450.

In another embodiment, the contamination detection workflow 1400 may use the machine learning engine 220 and training module 1455 to improve contamination detection. Various training datasets 1456 (e.g., parameters from parameter database 230, sequences from sequence database 210, etc.) may be used to supply information to the machine learning engine 220 as described herein. In accordance with this embodiment, the machine learning engine 220 may be used to train a contamination noise baseline to identify a noise threshold, determine a contamination level, determine a contamination event, and determine the limit of detection (LOD) for contamination detection. Additionally, machine learning engine may be used to calculate the sensitivity (true positive rate) and specificity (true negative rate) for contamination detection. That is, machine learning engine 220 can analyze different statistical significance indicators (such as p-values) and determine the threshold that achieves highest sensitivity at the minimum desired specificity level (e.g. 99%) for determining a contamination event.

Single sample component 1410 of contamination detection workflow 1400 is, for example, a runnable script that is used to estimate contamination in a sample. By contrast, baseline batch component 1430 of contamination detection algorithm 110 is, for example, a runnable script that is used for generating estimates across a batch of samples, and may also be used to generate a background noise model across these samples. The noise model is generated from a batch of samples previously determined to be healthy.

### VIII. Detecting Contamination Using Maf and Noise

Exemplary methods for using regression analysis for detecting contamination are described in PCT/IB2018/050979, which is incorporated herein by reference its entirety.

In one embodiment, the contamination detection workflow 1400 may be based on a model for estimating contamination. In one example, the model is a linear regression model based on population mean allele frequencies of the one or more pre-determined SNPs, herein referred to as the “population model” for clarity, that is configured for detecting contamination in sequencing data from a sample (e.g., a plurality of sequencing reads).

In one example, the population model determines contamination by calculating a probability that the observed variant frequency VAF for a sample (e.g., a plurality of sequencing reads) is statistically significant relative to the population mean allele frequency MAF and a background noise baseline. That is, the population model calculates a probability of observing a variant allele frequency VAF of a sample at a given contamination level α of the average minor allele frequency MAF of the population for any one or more of the pre-determined SNPs. If the population model determines that the observed VAF for the sample at a given contamination level α is above a threshold contamination level and statistically significant, the contamination detection workflow 1400 can call a contamination event.

In some embodiments, the population model can be informed by a sample call file (e.g., single variant call file 1412), a population call file (e.g., MAF call file 1414), and a set of variant call files (e.g., multiple variant call files 1422). The single variant call file 1412 includes, at least in part, observed variant allele frequencies VAFs for each of the one or more of the pre-determined SNPs that are present in the plurality of sequencing reads. Similarly, the population call file includes the minor allele frequencies of a population of test samples (MAFp). The minor allele frequency of the population of test samples MAFp can include the minor allele frequencies MAF of any number of SNPs of the population at any number of sites k. The set of variant call files includes the variant allele frequencies for a set of test samples (VAFB). The set of variant allele frequencies for a set of test samples can include variant allele frequencies VAF of any number of SNPs at any number of sites k.

### VIII. A Regression Model for MAF and Noise

In one embodiment, a contamination detection workflow 1400 determines a likelihood that a sample is contaminated using observed sequencing data and a background noise model. In some examples, the observed sequencing data can be included in a test sample call file (such as single variant call file 1412) and a population call file (such as MAF call file 1414). The background noise model can use a set of variant call files (such as multiple variant call files 1422) to determine a background noise baseline. Here, for the purpose of example, the probability of contamination for a single SNP is based on the relationship between a sample's observed variant allele frequency VAFs of the one or more pre-determined SNPs present in the sample, a population minor allele frequency MAFp, and a background noise baseline generated from a set of variant allele frequencies VAFB.

In one embodiment, the contamination detection workflow 1400 uses a population model on a sample including a number of SNPs, including one or more of the pre-determined SNPs. The population model can be represented as:

$$V A {F}_{S} = α MA {F}_{P} + β N ( V A {F}_{B} ) + ϵ \qquad ( 12 )$$

where α is the contamination level, β is the noise fraction for the sample (i.e., number of noisy SNPs over number of non-noisy SNPs), N is the background noise model based on a set of observed variant allele frequencies VAFB, and & is a random error term determined by the regression.

In some cases, the observed variant allele frequency of the sample VAFs and the minor allele frequency MAFp of the population can include a negated variant allele frequency VAF and a negated minor allele frequency (MAF). Negated variant allele frequencies and negated minor allele frequencies allow the data used by the population model to be similarly scaled such that data from homozygous alternate alleles and homozygous alleles in a test samples are similarly analyzed in the population model.

In one example embodiment, the population model includes each pre-determined SNP i in a sample. Each pre-determined SNP i of the test sample is associated with a site k (i.e., genomic position) and any number of reads of the test sample can be associated with site k. Therefore, each SNP i of a test sample has an observed variant allele frequency VAF associated with its site k. Further, each pre-determined SNP i at site k is associated with a minor allele frequency MAF for that site k. The minor allele frequency MAF for site k is the minor allele frequency MAF for reads from multiple samples at site k. For example, a first SNP i₁ of a test sample is associated with a first site k₁. The variant allele frequency VAF for the site k₁ is determined to be 0.03 from 1235 reads in the test sample associated with the first site k₁. The minor allele frequency MAF at the first site k₁ associated with the SNP i₁ is determined to be 0.01 from 1.108 SNPs in the population. A second SNP i₂ of a test sample is associated with a second site k₂. The variant allele frequency VAF for the site k₂ is determined to be 0.81 from 1792 reads in the test sample associated with the site k₂. The minor allele MAF frequency at site k₂ associated with the SNP i₂ at the site k₂ is determined to be 0.90 from 1.10⁹ SNPs in the population.

Therefore, the variant allele frequency of the test sample VAFs can be represented as:

$${VAF}_{S} = {\sum }_{k} {\sum }_{i} V A {F}_{k}^{i} \qquad ( 13 )$$

where VAF_{S} is the variant allele frequency of the test sample, the summation over k indicates that the variant allele frequency VAF_{S} includes the variant allele frequency of SNPs at all sites k included in the test sample, and the summation over i indicates that the variant allele frequency VAF at site k includes all SNPs i at site k. Similarly, the minor allele frequency of the population MAF_{P} can be represented as:

$$M A {F}_{P} = {\sum }_{k} {\sum }_{i} M A {F}_{k}^{i} \qquad ( 14 )$$

where MAF_{P} is the minor allele frequency of the population, the summation over k indicates that the minor allele frequency MAF includes the minor allele frequency MAF of SNPs of the population at all sites k included in the test sample, and the summation over i indicates that there is a minor allele frequency MAF associated with each SNP i at a site k of the test sample.

In one example embodiment, for a given test sample, there are three possible observed genotypes for each SNP i at a site k possible: homozygous reference 0/0, heterozygous 0/1, and homozygous alternative 1/1, where 0 represents the reference allele and 1 the alternative allele. In an uncontaminated test sample, the variant allele frequency values observed are expected to be close to 0, 0.5 and 1 for genotypes 0/0, 0/1 and 1/1, respectively. However, in a contaminated sample, the variant allele frequency values can be expected to shift from 0, 0.5, and 1, as the SNPs vary across the population, and thus, have a higher likelihood of being present in a contaminating sample. Modifying the variant allele frequencies VAF of the homozygous reference and homozygous alternative alleles such that the population model can analyze all genotypes of a test sample is beneficial.

Therefore, in some embodiments, the population model can, for some SNPs i, negate variant allele frequencies VAF for some SNPs such that the population model can more easily process the variant allele frequency VAF data. In one example embodiment, the variant allele frequency VAF for SNPs i at site k (VAFₖ+) included in the test sample can be described by:

$$V A {F}_{k}^{i} = \{ {VAF}_{k} if 0 < V A {F}_{k} < 0.2 NA if 0.2 \leq V A {F}_{k} \leq 0.81 - V A {F}_{k} if 0.8 < V A {F}_{k} < 1. \qquad ( 15 )$$

where VAFₖⁱ is the variant allele frequency VAF for an SNP i at site k of the test sample, VAFₖ is the variant allele frequency of all SNPs of the test sample at site k, and NA indicates that a SNP will not be considered. Here, the variant allele frequency VAF for SNP i at site k of the test sample (VAFₖ) is the determined variant allele frequency for the SNPs at site k (VAFₖ) if the SNP i is a homozygous reference genotype call. A homozygous reference call is a reference call with a variant allele frequency VAF of SNPs at site k greater than 0.0 and less than 0.2 (0<VAFₖ<0.2). The variant allele frequency for an SNP i at site k of the test sample (VAFₖⁱ) is not considered (marked as “NA” above) if the SNP i is a heterozygous reference genotype call. A heterozygous reference call is a reference call with a variant allele frequency VAF of SNPs at site k greater or equal to than 0.2 and less than or equal to 0.8 (0.2≤VAFₖ≤0.8). Finally, the variant allele VAF frequency for an SNP i at site k of the test sample (VAFₖⁱ) is 1 less the determined variant allele frequency VAFₖ for all the SNPs at site k if the SNP i is a homozygous alternative reference call. A homozygous alternative reference call is a reference call with a variant allele frequency VAF of SNPs at site k greater than 0.8 and less than 1.0 (0.8<VAFₖ<1.0).

In some embodiments, the population model can, for some SNPs i, negate the minor allele frequencies MAF based on the variant allele frequency for an SNP i at site k such that the population model can more easily process the data. For example, the minor allele frequency for an SNP i at site k can be described by:

$$M A {F}_{k}^{i} = \{ {MAF}_{k} if 0 < V A {F}_{k} < 0.2 NA if 0.2 \leq V A {F}_{k} \leq 0.81 - M A {F}_{k} if 0.8 < V A {F}_{k} < 1. \qquad ( 16 )$$

where MAFₖⁱ is the minor allele frequency MAF associated with SNP i at site k of the test sample, MAFₖ is the minor allele frequency of population SNPs at site k, NA indicates that a SNP will not be considered, and VAFₖ is the variant allele frequency of the SNPs of the test sample at site k. Here, the minor allele frequency MAF associated with SNP i at site k of the test sample (MAFₖⁱ) is the minor allele frequency for the SNPs of the population at site k (MAFₖ) if the SNP i is a homozygous reference genotype call. The minor allele frequency for a SNP i at site k of the test sample (MAFₖⁱ) is not considered (NA) if the SNP i is a heterozygous reference genotype call. Finally, the minor allele frequency associated with an SNP i at site k of the test sample (MAFₖⁱ) is the 1 less the determined minor allele frequency MAFₖ for all the SNPs at site k if the SNP i is a homozygous alternative reference call.

The population model can also include a background noise model N based on the variant allele frequencies from a set of variants (VAFB). The background noise model N can be used to distinguish a background noise baseline that is generated during sequencing of each SNP, such as, for example, during processes 100 and 300. The introduced noise may be from the sequence context of a variant and, therefore, some sites k will have a higher noise level and some sites k will have a lower noise level. Generally, the noise model is the average variant allele frequency for healthy variants of the set of variants at a given site k. Therefore, a given SNP i at site k of the sample can be associated with a background noise baseline associated with the site k. The background noise model N can determine a noise coefficient β representing the expected background noise baseline of each SNP.

In one approach, the population model regresses the contamination level α against the variant allele frequency for a test sample VAF_{S}, the minor allele frequency for the population MAF_{P}, and the background noise model N. That is, contamination detection workflow 1400 calculates a contamination level α of a sample using the associated observed variant allele frequency VAF, minor allele frequency MAF, and background noise model N for the pre-determined SNPs present in the sample. Contamination detection workflow 1400 determines a p-value of the contamination fraction α using the regression model across all pre-determined SNPs of a test sample. Based on the p-value and the contamination level α, the contamination detection workflow 1400 can determine that the sample is contaminated. For example, in one embodiment, if the determined contamination level α is above a threshold contamination value (e.g., 3%) and the p-value is below a threshold p-value (e.g., 0.05) the sample can be called contaminated.

In an alternative approach, the population model can calculate two contamination levels using the variant allele frequencies VAF and minor allele frequencies MAF of the pre-determined SNPs in the test sample. In one example, the population model can include a first regression including a first contamination level α₁ using SNPs with homozygous alternative reference calls and a second regression including a second contamination level α2 using SNPs with homozygous reference calls. If a significant regression p-value is observed from both regressions, contamination detection workflow 1400 can determine that the sample is contaminated. In this case, using two regression equations to detect a contamination event provides stronger evidence for contamination than a single regression equation.

### IX. Detecting Contamination Using Contamination Probability and Noise

Exemplary methods for using contamination probability and noise models for detecting contamination are described in PCT/IB2018/050979, which is hereby incorporated by reference in its entirety.

In another example embodiment of contamination detection workflow 1400 and the methods described herein, the contamination model for detecting contamination is a linear regression model based on a contamination probability generated from population mean allele frequencies, herein referred to as a “probability model” for convenience of description and delineation from the “population model” discussed previously. The probability model determines contamination by calculating a probability that the observed variant allele frequency for a plurality of sequencing read is statistically significant relative to a contamination probability and background noise baseline. That is, the probability model calculates a probability of observing a variant allele frequency VAF of a in a plurality of sequencing reads at a given contamination level alpha of the probable contamination frequency generated from the population. If the population model determines that the observed VAF for the test sample at a given contamination level α is above a threshold contamination level and statistically significant, the detection workflow 1400 can determine a contamination event.

In some embodiments, the probability model is informed by a test sample call file (e.g., single variant call file 1412), a population call file (e.g., MAF call file 1414), and a set of variant call files (e.g., multiple variant call files 1422). The test sample call file includes the observed variant allele frequencies VAF_{S} for a single test sample. The variant allele frequency of the test sample VAF_{S} can include observed variant allele frequencies VAF of each of the one or more pre-determined SNPs. Similarly, the population call file includes the minor allele frequencies MAF_{P} of a plurality of sequencing reads. The minor allele frequency of the plurality of sequencing reads MAF_{P} can include the minor allele frequencies of each of the one or more pre-determined SNPs. The set of variant call files includes the variant allele frequencies for a set of samples (i.e., different pluralities of sequencing reads), i.e. VAF_{B}. The set of variant allele frequencies for a set of samples can include variant allele frequencies at each of the one or more pre-determined SNPs.

### IX.A Regression Model for Contamination Probability and Noise

In one embodiment, a contamination detection workflow 1400 determines a likelihood that a sample is contaminated using observed sequencing data and a background noise model. In some examples, the observed sequencing data can be included in a sample call file (such as single variant call file 1412) and a population call file (such as MAF call file 1414). The background noise model can be used from a set of variant call files (such as multiple variant call files 1422) to determine a background noise baseline. Here, for the purpose of example, the probability of contamination for a single pre-determined SNP is based on the relationship between a sample's (i.e., plurality of sequencing reads) variant allele frequency VAF_{S}, a contamination probability C based on a population minor allele frequency MAF_{P}, and a background noise baseline generated from a set of variant allele frequencies VAF_{B}.

In one embodiment, the contamination detection workflow 1400 uses a population model on a test sample including a number of SNPs. The population model can be represented as:

$$V A {F}_{S} = α C ( M A {F}_{P} ) + β N ( V A {F}_{B} ) + ϵ \qquad ( 17 )$$

where C is contamination probability based on the minor allele frequency of the population MAF_{P}, α is the contamination level for the population, β is the noise fraction for the test sample, N is the background noise model generating a background noise baseline from the variant allele frequencies for a set of variants VAF_{B}, and ε is a random error term determined by the regression.

Here, the variant allele frequency of the test sample VAF_{S} and the minor allele frequency of the population MAF_{P} are similarly defined as in Eqns. 2 and 3. That is, each SNP i of the test sample is associated with a site k and the variant allele frequency for an SNP i is the variant allele frequency based on all SNPs at site k in the test sample. Further, each SNP i of the test sample is associated with a minor allele frequency MAF of all SNPs of the population at site k.

In some embodiments, contamination detection workflow 1400 uses a probability model based on the population minor allele frequency MAF_{P}. Therefore, the contamination probability associated with each SNP i at site k of the test sample can be represented as:

$$C ( M A {F}_{k}^{i} ) = {C}_{k}^{i} = {\sum }_{k} {\sum }_{i} {C}_{k}^{i} \qquad ( 18 )$$

where Cki is the contamination probability associated with each SNP i at site k of the test sample, the summation over k indicates that the contamination probability C includes the minor allele frequency MAF of SNPs of the population at all sites k included in the test sample, and the summation over i indicates that there is a contamination probability C associated with each SNP i of the test sample.

The contamination probability represents the likelihood a sample is contaminated based on the minor allele frequency MAF and genotype of the SNP i at site k. In one example embodiment, contamination probability C for an SNP i at site k (Cₖⁱ) included in the test sample can be described as:

$${C}_{k}^{i} = \{ 1 - {( 1 - M A {F}_{k} )}^{2} if 0 < V {F}_{k} < 0.2 NA if 0.2 \leq V {F}_{k} \leq 0.81 - {( M A {F}_{k} )}^{2} if 0.8 < V {F}_{k} < 1. \qquad ( 19 )$$

where Cₖⁱ is the probability of contamination probability C associated with SNP i at site k of the test sample, MAFₖ is the minor allele frequency of population SNPs at site k, NA indicates that an SNP will not be considered, and VAFₖ is the variant allele frequency of the SNPs of the test sample at site k. Here, the contamination probability C associated with SNP i at site k of the test sample (Cₖⁱ) is one less the quantity one less the minor allele frequency for SNPs of the population at site k squared (1-(1-MAFₖ)²) if the SNP i is a homozygous reference genotype call. The contamination probability for an SNP i at site k of the test sample (Cₖⁱ) is not considered (marked as “NA” above) if the SNP i is a heterozygous reference genotype call. Finally, the contamination probability C associated with SNP i at site k of the test sample (Cₖⁱ) is one less the quantity one less the minor allele frequency for SNPs of the population at site k squared (i.e., 1-(1-MAFₖ)²) if the SNP i is a homozygous reference genotype call.

In some embodiments, the probability model can include a background noise model N similar to the noise model described for detection workflow 1400. That is, the noise model is the average variant allele frequency for healthy variants of the set of variants at a given site k (i.e., VAF_{B}). Therefore, a given SNP i at site k of the test sample can be associated with a background noise baseline associated with the site k. The background noise model N can determine a noise coefficient β representing the expected background noise baseline of each SNP.

In this example, the probability model regresses the contamination level α against the variant allele frequency for a test sample VAF_{S}, the contamination probability C and the background noise model N. That is, contamination detection workflow 1400 calculates a contamination level α of a test sample using the associated variable allele frequency VAF, contamination probability C, and background noise model N for the SNPs of the test sample. Contamination detection workflow 1400 determines a p-value of the contamination fraction a of the SNPs in a test sample using the probability model. Based on the p-value and the contamination level α, the contamination detection workflow 1400 can determine that the test sample is contaminated. For example, in one embodiment, if the determined contamination fraction a is above a threshold contamination value (such as, for example, 3%) and the p-value is below a threshold p-value (such as, for example, 0.05) the sample can be called contaminated.

### X. Method of Pre-Detecting Presence of a Disease

In another aspect, this disclosure provides a method of predicting presence of a disease in a sample using, in part, the contamination detection methods described herein. In some cases, the disease is cancer. In some embodiments, the method of predicting presence of a disease in a sample includes: obtaining a plurality of sequencing reads for a plurality of nucleic acid fragments isolated from a sample comprising cell-free RNA (cfRNA); identifying contamination in a sample using any of the contamination detection methods described herein; and identifying SNPs from the plurality of sequencing reads that are informative for the presence of the disease.

In some embodiments, the methods of predicting presence of a disease include discarding a sample following determination that the sample is contaminated. In some embodiments, the method of predicting presence of a disease include assessing the risk introduced by contamination and using the risk in determining whether the sample is discarded. In some embodiments, the risk introduced by the contamination is determined in part by determining a likely source of contamination. In some embodiments, determining the contamination source lowers the risk introduced by the contamination, and wherein not determining the contamination source increases the risk introduced by the contamination.

### XI. Additional Considerations

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments of the invention may also relate to a product that is produced by a computing process described herein. Such a product may include information resulting from a computing process, where the information is stored on a non-transitory, tangible computer-readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method for identifying contamination in a sample, comprising:
(a) obtaining a plurality of sequencing reads for a plurality of nucleic acid fragments isolated from a sample comprising cell-free RNA (cfRNA);
(b) identifying sequencing reads that comprise one or more pre-determined single nucleotide polymorphisms (SNPs), thereby determining an observed allele frequency for each pre-determined SNP in the plurality of sequencing reads, wherein
each of the one or more pre-determined SNPs are selected from:
an allele present in one or more selected databases; or
a genotyping SNP associated with a sample type; and
(c) determining whether the sample is contaminated using a determined contamination probability of the one or more pre-determined SNPs.

2. The method of claim 1, wherein the identified sequencing reads that comprise the one or more pre-determined SNPs comprise a sequencing depth of at least 10 reads per million mapped reads (RPM).

3. The method of claim 1 or 2, wherein the identified sequencing read comprising the one or more pre-determined SNPs each comprise an exonic sequence.

4. The method of claim 3, wherein the exonic sequence comprises an exon-exon junction.

5. The method of any one of claims 1-4, wherein the allele present in one or more select databases comprises an allele present in a universal human reference database.

6. The method of claim 5, wherein the one or more pre-determined SNPs are selected from Table 1.

7. The method of any one of claims 1-6, wherein the allele present in the one or more select databases comprises an allele present in a NCBI dbSNP database (Build 155) that has a reference allele frequency in a range between 0.2 and 0.7.

8. The method of claim 7, wherein the one or more pre-determined SNPs are selected from Table 2.

9. The method of claim 8, wherein the one or more pre-determined SNPs does not include a conversion type comprising: A>G; T>C; C>T; or G>A.

10. The method of any one of claims 1-9, wherein the one or more pre-determined SNPs are selected from Table 3.

11. The method of any one of claim 1-10, further comprising determining a contamination probability for each pre-determined SNP using its observed allele frequency.

12. The method of any one of claims 1-11, further comprising identifying two or more pre-determined SNPs in the sequencing reads, thereby determining an observed allele frequency for each of the two or more pre-determined SNPs in the plurality of sequencing reads.

13. The method of claim 12, wherein the two or more pre-determined SNPs are selected from Table 1, Table 2, Table 3, or any combination thereof.

14. The method of any one of claims 1-13, wherein the allele present in a Universal Human Reference (UHR) comprises an allele having a homozygous frequency of at least 75% in the UHR and a homozygous frequency of 5% or less in a human sample.

15. The method of any one of claims 1-14, wherein the reference allele frequency is in a range between 0.3 and 0.7.

16. The method of any one of claims 1-15, wherein the reference allele frequency comprises a MAF, a VAF, a sequencing depth, or any combination thereof.

17. The method of claim 16, wherein the reference allele frequency comprises a MAF, wherein the MAF is in a range between 0.3 and 0.7.

18. The method of claim 1, further comprising filtering the sequences by removing sequencing reads comprising SNPs including no-calls prior to determining a contamination probability.

19. The method of claim 18, wherein filtering further comprises removing sequences having a SNP with a A>G; G>A; T>C; or C>T conversion.

20. The method of any one of claims 1-19, wherein the observed allelic frequency comprises:
a minor allele frequency (MAF), a variable allele frequency, a sequencing depth, a noise rate, or any combination thereof.

21. The method of any one of claims 1-20, wherein the observed allelic frequency comprises a MAF indicating contamination.

22. The method of claim 21, wherein the MAF is 0.5 or greater.

23. The method of any one of claims 1-22, further comprising discarding the sample following a determination that the sample is contaminated.

24. The method of any one of claims 1-22, further comprising assessing a risk introduced by contamination and using the risk in determining whether the sample is discarded.

25. The method of claim 24, wherein the risk introduced by the contamination is determined in part by determining a likely source of contamination.

26. The method of claim 25, wherein determining the contamination source lowers the risk introduced by the contamination, and wherein not determining the contamination source increases the risk introduced by the contamination.

27. The method of any one of claims 1-26, further comprising applying a contamination model to the sequencing reads identified as having one or more pre-determined SNPs and an observed allele frequency in the plurality of sequencing reads.

28. The method of any one of claims 1-27, wherein the contamination model comprises at least one likelihood test.

29. The method of claim 28, wherein one or more likelihood tests are applied to a sequencing read of the plurality of sequencing reads using the associated contamination probability, wherein each test to obtain a current contamination probability is indicative of whether the sequencing reads are contaminated.

30. The method of claim 28 or 29, further comprising:
determining that the sequencing reads are contaminated based on the current contamination probability of the at least one test being above a threshold associated with the at least one test likelihood test.

31. The method of any one of claims 28-30, further comprising:
determining that the sequencing reads are contaminated based on the current contamination probability of at least two likelihood tests being above a threshold associated with the at least two likelihood tests.

32. The method of any one of claims 28-31, wherein the at least one likelihood test maximizes a likelihood function, the likelihood function proportional to the probability of an event occurring in a data set given a variable.

33. The method of any of claims 28-32, wherein applying the at least one likelihood test of the contamination model comprises:
comparing a set of generated contaminated sequencing reads to a set of previously obtained non-contaminated sequencing reads to determine the contamination probability.

34. The method of any one of claims 28-33, wherein applying at least one likelihood test of the contamination model comprises:
generating a null hypothesis representing that the sequencing reads are not contaminated;
generating a set of contamination hypotheses representing that the sequencing reads are contaminated, wherein each contamination hypothesis of the set of contamination hypotheses is contaminated at a different contamination level; and
applying a likelihood ratio test between the set of contamination hypotheses and the null hypothesis, wherein the likelihood ratio test obtains the current contamination probability.

35. The method of any one of claims 28-34, wherein applying the at least one likelihood test of the contamination model comprises:
comparing a set of generated contaminated sequencing reads to an average of previously obtained sequencing reads to determine the contamination probability, wherein the contamination probability is associated with the likelihood that the sequencing reads are contaminated at a contamination level.

36. The method of any one of claims 28-35, wherein applying at least one likelihood test of the contamination model comprises:
generating a set of contamination hypotheses representing that the sequencing reads are contaminated, wherein each contamination hypothesis of the set of contamination hypotheses is contaminated at a different contamination level;
generating a null hypothesis representing the mean minor allele frequency at a contamination level for a plurality of previously obtained sequencing reads, wherein the contamination level is associated with the contamination hypothesis most likely to be contaminated; and
applying a likelihood ratio test between the set of contamination hypotheses and the null hypothesis, wherein the likelihood ratio test obtains the current contamination probability.

37. The method of any one of claims 1-27, wherein the contamination model comprises generating a noise model.

38. The method of claim 37, wherein the noise model represents a measure of background noise in a subset of sequencing reads, and wherein the noise model is generated based on the subset of the sequencing reads.

39. The method of claim 37 or 38, further comprising applying the contamination model to an identified sequencing read using the observed allele frequency of the one or more pre-determined SNPs in the identified sequencing reads and the generated noise model to obtain a confidence score representing a measure of the predicted contamination in the sequencing reads.

40. The method of any one of claims 37-39, wherein the background noise is a population measure of allele frequency in the subset of sequencing reads.

41. The method of claim 40, wherein the background noise is representative of the static noise generated when sequencing a SNP.

42. The method of any of claims 38-41, wherein the subset of sequencing reads comprises SNPs from uncontaminated and healthy test samples.

43. The method of any of claims 37-42, wherein generating the noise model further comprises:
determining a noise coefficient for each SNP of the subset of sequencing reads, wherein the noise coefficient predicts the expected noise level for each SNP.

44. The method of any of claims 37-43, wherein the noise model generated based on the subset of sequencing reads is additionally based on a sample type of the sequencing reads.

45. The method of any of claims 37-44, wherein when the confidence score is above a threshold the contamination model predicts that the sequencing reads are contaminated.

46. The method of any of claims 37-45, wherein the contamination model additionally includes a random error term.

47. A system for determining contamination in a sample, comprising:
(a) a computer processor; and
(b) a non-transitory computer-readable storage medium storing instructions that, when executed by the computer processor, cause the computer processor to perform steps of any of the methods of claims 1-46.

48. A method of predicting presence of a disease in a sample, comprising:
(a) obtaining a plurality of sequencing reads for a plurality of nucleic acid fragments isolated from a sample comprising cell-free RNA (cfRNA);
(b) identifying contamination in a sample using any of the methods of claims 1-46; and
(c) identifying SNPs from the plurality of sequencing reads that are informative for the presence of a disease.

49. The method of claim 48, further comprising assessing the risk introduced by contamination identified in step (b).

50. The method of claim 49, wherein the risk introduced by the contamination is determined in part by determining a likely source of contamination.

51. The method of claim 50, wherein determining the contamination source lowers the risk introduced by the contamination, and wherein not determining the contamination source increases the risk introduced by the contamination.

52. The method of any one of claims 48-51, wherein a contaminated sample is discarded based in part on the presence of contamination, the risk introduced by the contamination, or both.

53. The method of claim 48, wherein the disease is cancer.
